SEQUENCE LISTING (29 sequences)

SEQ ID NO: 1 - DNA, 2494 bp, Homo Sapiens
gagaaaggagagaaaggaaagcgcgaggagccgccgccaccaccagcgcagcagtcctggagctgtgaggagattcgggccgtcaccctgcctcccctgcgtcccgccaccggccgcttctgtcctcggacccattccaacaatctcgtaaaacatggtggattactatgaagttctaggcgtgcagagacatgcctcacccgaggatattaaaaaggcatatcggaaactggcactgaagtggcatccagataaaaatcctgagaataaagaagaagcagagagaaaattcaagcaagtagcggaggcatatgaagtgctgtcggatgctaagaaacgggacatctatgacaaatatggcaaagaaggattaaatggtggaggaggaggtggaagtcattttgacagtccatttgaatttggcttcacattccgtaacccagatgatgtcttcagggaattttttggtggaagggacccattttcatttgacttctttgaagacccttttgaggacttctttgggaatcgaaggggtccccgaggaagcagaagccgagggacggggtcgtttttctctgcgttcagtggatttccgtcttttggaagtggattttcttcttttgatacaggatttacttcatttgggtcactaggtcacgggggcctcacttcattctcttccacgtcatttggtggtagtggcatgggcaacttcaaatcgatatcaacttcaactaaaatggttaatggcagaaaaatcactacaaagagaattgtcgagaacggtcaagaaagagtagaagttgaagaagatggccagttaaagtccttaacaataaatggtgtggccgacgacgatgccctcgctgaggagcgcatgcggagaggccagaacgccctgccagcccagcctgccggcctccgcccgccgaagccgccccggcctgcctcgctgctgagacacgcgcctcactgtctctctgaggaggagggcgagcaggaccgacctcgggcacccgggccctgggaccccctcgcgtccgcagcaggattgaaagaaggtggcaagaggaagaagcagaagcagagagaggagtcgaagaagaagaagtcgaccaaaggcaatcactagaccggacttgaggcacgcggtgcacccccagacgctggcgctccaccgtgctcggcatgcggtcgtgcacacgcgctaggtagcagcgtcggtcaggactgtctcgaggccacactcgctcggcaggattatgcgatcacggatcagtcagagcagggtcaggagacggggctgacggcacgggtggcggggacagacgtttgggacttggccgcgactctctgcttctctccagctctcaatctgctgcattttcctctagtgcttccggatcctcttcattcttttcggctactcaaccactccgcatgctgctggaatatttctggctttagaagtacaggagggcgcagatggctaactgagtaacattcatgaaatgaggctttctgtggcggcgtagtgtttggaattagaaggtaattcagtagagtgtaacttagagaatattgcaagtgacacattgaatcctgcccgtcagggcaccttttcctcagagcaatccggccacacgaatagaaggctgtcgtgaatcacatcagatgtaaaatcattccttctgtttactcttttaattttcatcctttgcaggtagtgcaaattcaacttcaaatatggtgtaggttttgctagattccatatttttttcttggatttttgctaattatttttagcaaaaaatttttgctcagtggcactctccctagtgtccatgggttagggccatgctggggaaaacgggccggtatttacacacgcgcaaaacacccagagacggcacaaggaggttgaactcatgtttcagttcgcgaacattgactccttacgaaagtcacttcattctaactagatgcgcccacttccggtcattatttcgtttgcatgatgtattgcttcttcacgttttgtttttattgagcacggagtagaattccagggctgccttgacttcttccctgcatgctccctcccagtgactttccttccctttcacatgaggatctgccgttcatgttgctttctcctttgtcctcttggacttgagggcattgtgaaaagctttgctgtgatttaaaaatgccagcaattttaatctagcagtgttgaagctgggaattttttggcgcaatccatgtagcagtgacccaggcttgggagccagaaacaagtgtgacctgggattttatttaacacaactgttgccaaagagttggctttgtttatttggttttggcggggagaggagtggtatttgatgctttctgtggacaatgtaaccctaaacacatcatgtattttaaatgccacctacataaataaaacataagcatattgaatacaaaaaaaaaaaaaaaaaaa

SEQ ID NO: 2 - protein, 326 aa, Homo Sapiens
MVDYYEVLGVQRHASPEDIKKAYRKLALKWHPDKNPENKEEAERKFKQVAEAYEVLSDAKKRDIYDKYGKEGLNGGGGGGSHFDSPFEFGFTFRNPDDVFREFFGGRDPFSFDFFEDPFEDFFGNRRGPRGSRSRGTGSFFSAFSGFPSFGSGFSSFDTGFTSFGSLGHGGLTSFSSTSFGGSGMGNFKSISTSTKMVNGRKITTKRIVENGQERVEVEEDGQLKSLTINGVADDDALAEERMRRGQNALPAQPAGLRPPKPPRPASLLRHAPHCLSEEEGEQDRPRAPGPWDPLASAAGLKEGGKRKKQKQREESKKKKSTKGNH

SEQ ID NO: 3 - DNA, 1568 bp, Homo Sapiens
ggagaaaggagagaaaggaaagcgcgaggagccgccgccaccaccagcgcagcagtcctggagctgtgaggagattcgggccgtcaccctgcctcccctgcgtcccgccaccggccgcttctgtcctcggacccattccaacaatctcgtaaaacatggtggattactatgaagttctaggcgtgcagagacatgcctcacccgaggatattaaaaaggcatatcggaaactggcactgaagtggcatccagataaaaatcctgagaataaagaagaagcagagagaaaattcaagcaagtagcggaggcatatgaagtgctgtcggatgctaagaaacgggacatctatgacaaatatggcaaagaaggattaaatggtggaggaggaggtggaagtcattttgacagtccatttgaatttggcttcacattccgtaacccagatgatgtcttcagggaattttttggtggaagggacccattttcatttgacttctttgaagacccttttgaggacttctttgggaatcgaaggggtccccgaggaagcagaagccgagggacggggtcgtttttctctgcgttcagtggatttccgtcttttggaagtggattttcttcttttgatacaggatttacttcatttgggtcactaggtcacgggggcctcacttcattctcttccacgtcatttggtggtagtggcatgggcaacttcaaatcgatatcaacttcaactaaaatggttaatggcagaaaaatcactacaaagagaattgtcgagaacggtcaagaaagagtagaagttgaagaagatggccagttaaagtccttaacaataaatggtaaggagcagctgctgcgcttggataacaagtaattcaacgcacgcacttaacagaaatgttaaactataacaagcaccatttgaggattaacaggaacatttttttgaagatttcaaacgaactcgactttcagtataattgtacctaaagtatttataaacagctcatcggagcctctatttgtcatagacttttgagttgattgttgggaccacataataggaccattttttttttgtctttaaaattgttgtaaatctctgtatgcactttgcttttttattaaacgtactccaaggtgagtcttgactctttagtgtaggacaagattgtacactaacaccagcatggacctgcttttcattgtgtctgaaatgtgagccacgtagtgtcggcctgctgtgaagttaacattgccaggacgattcttctacagaaataatttcaatttttttcagtatttagtagtgaaagatattaatacattaatggtaatacatttctggtttaatataaattaaggatgttttctagttgtgcatgaatgctggcaacttagtaagttttgacaattgtttaaatatgtaatgttaagcttaggtttaaaaaagtaaagctggtaaactgggtctttgtcatttgctttaaaaaaaaaaaaaaagaaaataaatgcgaatgtgttggtgcattcttcctgagtgggatctggaaaaaaaaaa

SEQ ID NO: 4 - protein, 241 aa, Homo Sapiens
MVDYYEVLGVQRHASPEDIKKAYRKLALKWHPDKNPENKEEAERKFKQVAEAYEVLSDAKKRDIYDKYGKEGLNGGGGGGSHFDSPFEFGFTFRNPDDVFREFFGGRDPFSFDFFEDPFEDFFGNRRGPRGSRSRGTGSFFSAFSGFPSFGSGFSSFDTGFTSFGSLGHGGLTSFSSTSFGGSGMGNFKSISTSTKMVNGRKITTKRIVENGQERVEVEEDGQLKSLTINGKEQLLRLDNK

SEQ ID NO: 5 - DNA, 1815 bp, Homo Sapiens
gcagagctctgtgctccctgcagtcaggactctgggaccgcagggggctcccggaccctgactctgcagccgaaccggcacggtttcgtggggacccaggcttgcaaagtgacggtcattttctctttctttctccctcttgagtccttctgagatgatggctctgggcgcagcgggagctacccgggtctttgtcgcgatggtagcggcggctctcggcggccaccctctgctgggagtgagcgccaccttgaactcggttctcaattccaacgctatcaagaacctgcccccaccgctgggcggcgctgcggggcacccaggctctgcagtcagcgccgcgccgggaatcctgtacccgggcgggaataagtaccagaccattgacaactaccagccgtacccgtgcgcagaggacgaggagtgcggcactgatgagtactgcgctagtcccacccgcggaggggacgcaggcgtgcaaatctgtctcgcctgcaggaagcgccgaaaacgctgcatgcgtcacgctatgtgctgccccgggaattactgcaaaaatggaatatgtgtgtcttctgatcaaaatcatttccgaggagaaattgaggaaaccatcactgaaagctttggtaatgatcatagcaccttggatgggtattccagaagaaccaccttgtcttcaaaaatgtatcacaccaaaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagcgttgttactgtggagaaggtctgtcttgccggatacagaaagatcaccatcaagccagtaattcttctaggcttcacacttgtcagagacactaaaccagctatccaaatgcagtgaactccttttatataatagatgctatgaaaaccttttatgaccttcatcaactcaatcctaaggatatacaagttctgtggtttcagttaagcattccaataacaccttccaaaaacctggagtgtaagagctttgtttctttatggaactcccctgtgattgcagtaaattactgtattgtaaattctcagtgtggcacttacctgtaaatgcaatgaaacttttaattatttttctaaaggtgctgcactgcctatttttcctcttgttatgtaaatttttgtacacattgattgttatcttgactgacaaatattctatattgaactgaagtaaatcatttcagcttatagttcttaaaagcataaccctttaccccatttaattctagagtctagaacgcaaggatctcttggaatgacaaatgataggtacctaaaatgtaacatgaaaatactagcttattttctgaaatgtactatcttaatgcttaaattatatttccctttaggctgtgatagtttttgaaataaaatttaacatttaatatcatgaaatgttataagtagacatacattttgggattgtgatcttagaggtttgtgtgtgtgtacgtatgtgtgtgttctacaagaacggaagtgtgatatgtttaaagatgatcagagaaaagacagtgtctaaatataagacaatattgatcagctctagaataactttaaagaaagacgtgttctgcattgataaactcaaatgatcatggcagaatgagagtgaatcttacattactactttcaaaaatagtttccaataaattaataatacctaaaaaaaaaaa

SEQ ID NO: 6 - protein, 266 aa, Homo Sapiens
MMALGAAGATRVFVAMVAAALGGHPLLGVSATLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 7 - DNA, 2586 bp, Homo Sapiens
ccagcgtgctgaagccggagcgagctagccgcccggagccgcgccgacccagctgagcccagcccacgggacgccagacctcgaccgtcgctcctaccccggccaccgctcggagccgaggcggacgcgtcccgatcttcccctgtccccaccctgccccgaccctcctctccacctctcgcgtcgtgacaccagctgtctccggcagcctcttggtcatgaaagccctcagattgtcggcttccgccctcttctgccttctgctgatcaacgggttaggggcagcaccccctggtcgccctgaggcgcagcctcctcctctcagctctgagcataaagagccggtagccggggacgcagtgcccgggccaaaggatggcagcgccccagaggtccgaggcgctcggaattccgagccgcaggacgagggagagcttttccagggcgtggatccccgggcgctggccgcggtgctgctgcaggcactcgaccgtcccgcctcacccccggcaccaagcggctcccagcaggggccggaggaagaagcagctgaagctctgctgaccgagaccgtgcgcagccagacccacagcctcccggcgccggagagcccggagcccgcggctccgcctcgccctcagactccggagaatgggcccgaggcgagcgatccctccgaggagctcgaggcgctagcgtccctgctccaggaactgcgagatttcagtccaagtagcgccaagcgccagcaggagacggcggcagcagagacggaaacccgcacgcacacgctgacccgagtgaatctggagagcccggggccagagcgcgtatggcgcgcttcctggggagagttccaggcgcgtgtcccggagcgcgcgcccctgccgcccccggccccctctcaattccaggcgcgtatgcccgacagcgggccccttcccgaaacccacaagttcggggaaggagtgtcctcccccaaaacacacctaggcgaggcattggcacccctgtccaaggcgtaccaaggcgtggccgccccgttccccaaggcgcgccggccggagagcgcactcctgggcggctccgaggcgggcgagcgccttctccagcaagggctggcgcaggtggaggccgggcggcggcaggcggaggccacgcggcaggccgcggcgcaggaagagcggctggccgacctcgcctcggacctgctgctccagtatttgctgcagggcggggcccggcagcgcggcctcgggggtcgggggctgcaggaggcggcggaggagcgagagagtgcaagggaggaggaggaggcggagcaggagagacgcggcggggaggagagggtgggggaagaggatgaggaggcggccgaggcggaggcagaggcggaggaggcggagagggcgcggcagaacgcgctcctgttcgcggaggaggaggacggggaagccggcgccgaggacaagcgctcccaggaggagacgccgggccaccggcggaaggaggccgaggggacagaggagggcggggaggaggaggacgacgaggagatggatccgcagacgatcgacagcctcattgagctgtccaccaaactccacctgccagcggacgacgtggtcagcatcatcgaggaggtggaggagaagcggaagcggaagaagaacgcccctcccgagcccgtgccgcccccccgtgccgcccccgcccccacccacgtccgctccccgcagcccccgccccccgcccccgctcccgcacgagacgagctgccggactggaacgaggtgctcccgccctgggatcgggaggaggacgaggtgtacccgccagggccgtaccaccctttccccaactacatccggccgcggacactgcagccgccctcggccttgcgccgccgccactaccaccacgccttgccgccttcgcgccactatcccggccgggaggcccaggcgcggcgcgcgcaggaggaggcggaggcggaggagcgccggctgcaggagcaggaggagctggagaattacatcgagcacgtgctgctccggcgcccgtgactgcccttcccggtcccgcccccgcgcgcccccgccgcgcgcgcgcgccggcgcccccctccgtgttgcccgctccccctcggtgtttgcatgcgccccggccctgccccttggccctgcccctgtccccgggctgcgtcgggacctgccagacccccctcccgggtcctgagcccgaactcccagagctcacccgcgggtgaccgggggccagcccaggagggcgggtggtttgtgcgagttcccttgccacgcggggccccggccccatcaagtccctctggggacgtccccgtcggaaaccggaaaaagcagttccagttaattgtgtgaagtgtgtctgtctccagcccttcgggcctcccacgagcccctccagcctctccaagtcgctgtgaattgaccccttctttcctttctctgttgtaaatacccctcacggaggaaatagttttgctaagaaataaaagtgactattttattaggaaaaaaaaaaaaaaaaaaaaaaaaaaaaaaaaaa

SEQ ID NO: 8 - protein, 615 aa, Homo Sapiens
MKALRLSASALFCLLLINGLGAAPPGRPEAQPPPLSSEHKEPVAGDAVPGPKDGSAPEVRGARNSEPQDEGELFQGVDPRALAAVLLQALDRPASPPAPSGSQQGPEEEAAEALLTETVRSQTHSLPAPESPEPAAPPRPQTPENGPEASDPSEELEALASLLQELRDFSPSSAKRQQETAAAETETRTHTLTRVNLESPGPERVWRASWGEFQARVPERAPLPPPAPSQFQARMPDSGPLPETHKFGEGVSSPKTHLGEALAPLSKAYQGVAAPFPKARRPESALLGGSEAGERLLQQGLAQVEAGRRQAEATRQAAAQEERLADLASDLLLQYLLQGGARQRGLGGRGLQEAAEERESAREEEEAEQERRGGEERVGEEDEEAAEAEAEAEEAERARQNALLFAEEEDGEAGAEDKRSQEETPGHRRKEAEGTEEGGEEEDDEEMDPQTIDSLIELSTKLHLPADDVVSIIEEVEEKRKRKKNAPPEPVPPPRAAPAPTHVRSPQPPPPAPAPARDELPDWNEVLPPWDREEDEVYPPGPYHPFPNYIRPRTLQPPSALRRRHYHHALPPSRHYPGREAQARRAQEEAEAEERRLQEQEELENYIEHVLLRRP

SEQ ID NO: 9 - DNA, 1247 bp, Homo Sapiens
gataatatctgtgcctcctgcccagaaccctccaagcagacacaatggtaagaatggtgcctgtcctgctgtctctgctgctgcttctgggtcctgctgtcccccaggagaaccaagatggtcgttactctctgacctatatctacactgggctgtccaagcatgttgaagacgtccccgcgtttcaggcccttggctcactcaatgacctccagttctttagatacaacagtaaagacaggaagtctcagcccatgggactctggagacaggtggaaggaatggaggattggaagcaggacagccaacttcagaaggccagggaggacatctttatggagaccctgaaagacatcgtggagtattacaacgacagtaacgggtctcacgtattgcagggaaggtttggttgtgagatcgagaataacagaagcagcggagcattctggaaatattactatgatggaaaggactacattgaattcaacaaagaaatcccagcctgggtccccttcgacccagcagcccagataaccaagcagaagtgggaggcagaaccagtctacgtgcagcgggccaaggcttacctggaggaggagtgccctgcgactctgcggaaatacctgaaatacagcaaaaatatcctggaccggcaagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcacaatggaaatggcacttaccagtcctgggtggtggtggcagtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagcccacccccaccccattgtctaatctgtagaagctaataaataatcatccctccttgcctagcataaaaaaaaaaaaaaaaaaaaaaaaaaaaaaaa

SEQ ID NO: 10 - protein, 298 aa, Homo Sapiens
MVRMVPVLLSLLLLLGPAVPQENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS

SEQ ID NO: 11 - DNA, 3178 bp, Homo Sapiens
gttgcctgtctctaaacccctccacattcccgcggtccttcagactgcccggagagcgcgctctgcctgccgcctgcctgcctgccactgagggttcccagcaccatgagggcctggatcttctttctcctttgcctggccgggagggccttggcagcccctcagcaagaagccctgcctgatgagacagaggtggtggaagaaactgtggcagaggtgactgaggtatctgtgggagctaatcctgtccaggtggaagtaggagaatttgatgatggtgcagaggaaaccgaagaggaggtggtggcggaaaatccctgccagaaccaccactgcaaacacggcaaggtgtgcgagctggatgagaacaacacccccatgtgcgtgtgccaggaccccaccagctgcccagcccccattggcgagtttgagaaggtgtgcagcaatgacaacaagaccttcgactcttcctgccacttctttgccacaaagtgcaccctggagggcaccaagaagggccacaagctccacctggactacatcgggccttgcaaatacatccccccttgcctggactctgagctgaccgaattccccctgcgcatgcgggactggctcaagaacgtcctggtcaccctgtatgagagggatgaggacaacaaccttctgactgagaagcagaagctgcgggtgaagaagatccatgagaatgagaagcgcctggaggcaggagaccaccccgtggagctgctggcccgggacttcgagaagaactataacatgtacatcttccctgtacactggcagttcggccagctggaccagcaccccattgacgggtacctctcccacaccgagctggctccactgcgtgctcccctcatccccatggagcattgcaccacccgctttttcgagacctgtgacctggacaatgacaagtacatcgccctggatgagtgggccggctgcttcggcatcaagcagaaggatatcgacaaggatcttgtgatctaaatccactccttccacagtaccggattctctctttaaccctccccttcgtgtttcccccaatgtttaaaatgtttggatggtttgttgttctgcctggagacaaggtgctaacatagatttaagtgaatacattaacggtgctaaaaatgaaaattctaacccaagacatgacattcttagctgtaacttaactattaaggccttttccacacgcattaatagtcccatttttctcttgccatttgtagctttgcccattgtcttattggcacatgggtggacacggatctgctgggctctgccttaaacacacattgcagcttcaacttttctctttagtgttctgtttgaaactaatacttaccgagtcagactttgtgttcatttcatttcagggtcttggctgcctgtgggcttccccaggtggcctggaggtgggcaaagggaagtaacagacacacgatgttgtcaaggatggttttgggactagaggctcagtggtgggagagatccctgcagaacccaccaaccagaacgtggtttgcctgaggctgtaactgagagaaagattctggggctgtgttatgaaaatatagacattctcacataagcccagttcatcaccatttcctcctttacctttcagtgcagtttcttttcacattaggctgttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctccccttttttcttctattaatcaagagaaacttcaaagttaatgggatggtcggatctcacaggctgagaactcgttcacctccaagcatttcatgaaaaagctgcttcttattaatcatacaaactctcaccatgatgtgaagagtttcacaaatccttcaaaataaaaagtaatgacttagaaactgccttcctgggtgatttgcatgtgtcttagtcttagtcaccttattatcctgacacaaaaacacatgagcatacatgtctacacatgactacacaaatgcaaacctttgcaaacacattatgcttttgcacacacacacctgtacacacacaccggcatgtttatacacagggagtgtatggttcctgtaagcactaagttagctgttttcatttaatgacctgtggtttaacccttttgatcactaccaccattatcagcaccagactgagcagctatatccttttattaatcatggtcattcattcattcattcattcacaaaatatttatgatgtatttactctgcaccaggtcccatgccaagcactggggacacagttatggcaaagtagacaaagcatttgttcatttggagcttagagtccaggaggaatacattagataatgacacaatcaaatataaattgcaagatgtcacaggtgtgatgaagggagagtaggagagaccatgagtatgtgtaacaggaggacacagcattattctagtgctgtactgttccgtacggcagccactacccacatgtaactttttaagatttaaatttaaattagttaacattcaaaacgcagctccccaatcacactagcaacatttcaagtgcttgagagccatgcatgattagtggttaccctattgaataggtcagaagtagaatcttttcatcatcacagaaagttctattggacagtgctcttctagatcatcataagactacagagcacttttcaaagctcatgcatgttcatcatgttagtgtcgtattttgagctggggttttgagactccccttagagatagagaaacagacccaagaaatgtgctcaattgcaatgggccacatacctagatctccagatgtcatttcccctctcttattttaagttatgttaagattactaaaacaataaaagctcctaaaaaatcaaaaaaaaaaaaaaaaaaaaaaaaaaaaaaaaaaaaaaaa

SEQ ID NO: 12 - protein, 303 aa, Homo Sapiens
MRAWIFFLLCLAGRALAAPQQEALPDETEVVEETVAEVTEVSVGANPVQVEVGEFDDGAEETEEEVVAENPCQNHHCKHGKVCELDENNTPMCVCQDPTSCPAPIGEFEKVCSNDNKTFDSSCHFFATKCTLEGTKKGHKLHLDYIGPCKYIPPCLDSELTEFPLRMRDWLKNVLVTLYERDEDNNLLTEKQKLRVKKIHENEKRLEAGDHPVELLARDFEKNYNMYIFPVHWQFGQLDQHPIDGYLSHTELAPLRAPLIPMEHCTTRFFETCDLDNDKYIALDEWAGCFGIKQKDIDKDLVI

SEQ ID NO: 13 - DNA, 731 bp, Homo Sapiens
gagaactcttgagaccgggagcccagctgcccaccctctggacattcacccagccaggtggtctcgtcacctcagaggctccgccagactcctgcccaggccaggactgaggcaagcctcaaggcacttctaggacctgcctcttctcaccaagatgaactcactggtttcttggcagctactgcttttcctctgtgccacccactttggggagccattagaaaaggtggcctctgtggggaattctagacccacaggccagcagctagaatccctgggcctcctggcccccggggagcagagcctgccgtgcaccgagaggaagccagctgctactgccaggctgagccgtcgggggacctcgctgtccccgccccccgagagctccgggagcccccagcagccgggcctgtccgccccccacagccgccagatccccgcaccccagggcgcggtgctggtgcagcgggagaaggacctgccgaactacaactggaactccttcggcctgcgcttcggcaagcgggaggcggcaccagggaaccacggcagaagcgctgggcggggctgagggcgcaggtgcggggcagtgaacttcagaccccaaaggagtcagagcatgcggggcgggggcggggggcggggacgtagggctaagggagggggcgctggagcttccaacccgaggcaataaaagaaatgttgcgtaactcaaaaaaaaaaaaaaaaaa

SEQ ID NO: 14 - protein, 138 aa, Homo Sapiens
MNSLVSWQLLLFLCATHFGEPLEKVASVGNSRPTGQQLESLGLLAPGEQSLPCTERKPAATARLSRRGTSLSPPPESSGSPQQPGLSAPHSRQIPAPQGAVLVQREKDLPNYNWNSFGLRFGKREAAPGNHGRSAGRG

SEQ ID NO: 15 - DNA, 1641 bp, Homo Sapiens
ctccctgtgttggtggaggatgtctgcagcagcatttaaattctgggagggcttggttgtcagcagcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagaatgctgtgtcctctgaagaaaccaatgactttaaacaagagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgcctaaaaaaaaaaaaaaaaa

SEQ ID NO: 16 - protein, 314 aa, Homo Sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 17 - DNA, 1616 bp, Homo Sapiens
ctccctgtgttggtggaggatgtctgcagcagcatttaaattctgggagggcttggttgtcagcagcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgcctaaaaaaaaaaaaaaaaaaaaaaaaaaaaaaaaaa

SEQ ID NO: 18 - protein, 300 aa, Homo Sapiens
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 19 - DNA, 1560 bp, Homo Sapiens
ctccctgtgttggtggaggatgtctgcagcagcatttaaattctgggagggcttggttgtcagcagcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagaatgctgtgtcctctgaagaaaccaatgactttaaacaagagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgcctaaaaaaaaaaaaaaaaa

SEQ ID NO: 20 - protein, 287 aa, Homo Sapiens
MRIAVICFCLLGITCAIPVKQADSGSSEEKQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 21 - DNA, 1373 bp, Homo Sapiens
gggaagcgctcgcgagatcttcagggtctatatataagcgcggggagcctgcgtcctttccctggtgtgattccgtcctgcgcggttgttctctggagcagcgttcttttatctccgtccgccttctctcctacctaagtgcgtgccgccacccgatggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgcctgtttagtttttaaagatggaactccaccctttgcttggttttaagtatgtatggaatgttatgataggacatagtagtagcggtggtcagacatggaaatggtggggagacaaaaatatacatgtgaaataaaactcagtattttaataaagtaaaaaaaaaaaaaaa

SEQ ID NO: 22 - protein, 294 aa, Homo Sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 23 - DNA, 1286 bp, Homo Sapiens
gggaagcgctcgcgagatcttcagggtctatatataagcgcggggagcctgcgtcctttccctggtgtgattccgtcctgcgcggttgttctctggagcagcgttcttttatctccgtccgccttctctcctacctaagtgcgtgccgccacccgatggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaagaaaatagtttaaacaatttgttaaaaaattttccgtcttatttcatttctgtaacagttgatatctggctgtcctttttataatgcagagtgagaactttccctaccgtgtttgataaatgttgtccaggttctattgccaagaatgtgttgtccaaaatgcctgtttagtttttaaagatggaactccaccctttgcttggttttaagtatgtatggaatgttatgataggacatagtagtagcggtggtcagacatggaaatggtggggagacaaaaatatacatgtgaaataaaactcagtattttaataaagtaaaaaaaaaaaaaaa

SEQ ID NO: 24 - protein, 265 aa, Homo Sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKGQESFKKQEKTPKTPKGPSSVEDIKAKMQASIEKGGSLPKVEAKFINYVKNCFRMTDQEAIQDLWQWRKSL

SEQ ID NO: 25 - DNA, 1274 bp, Homo Sapiens
gggaagcgctcgcgagatcttcagggtctatatataagcgcggggagcctgcgtcctttccctggtgtgattccgtcctgcgcggttgttctctggagcagcgttcttttatctccgtccgccttctctcctacctaagtgcgtgccgccacccgatggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaagcgcattgaacagtcctgggcactacatgtaaattaagcccaaagatggggagaaaggaaaaggagagacaaatatagtccatactgagtgtcatcaacaatccagactgaagtcttctattttaatctcaatccccttttctgatttgccacccatgcctcttcaggctggaaacaatctcttggttccctaaagcactttcttctgactgctgtgattcagtgaaccttgccctttgctttctattacttgtgcatttgcctcacctgacaatgttttaaatcgcctttgtatctccttagctgctcaataaatatttgaatgcatcaaaaaaaaaaaaaaaaaaa

SEQ ID NO: 26 - protein, 259 aa, Homo Sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKAH

SEQ ID NO: 27 - protein, 10 aa, Homo Sapiens
KEQLLRLDNK

SEQ ID NO: 28 - protein, 15 aa, Homo Sapiens
KRKKQKQREESKKKK

SEQ ID NO: 29 - protein, 15 aa, Homo Sapiens
RPDRPETTEESKKKK

(19) United States
(12) Patent Application Publication
(10) Pub. No.: US 2015/0252436 A1
(43) Pub. Date: Sep. 10, 2015

(54) METHODS AND COMPOSITIONS FOR THE DIAGNOSIS, PROGNOSIS AND TREATMENT OF CANCER

(71) Applicant: University of South Alabama, Mobile, AL (US)

(72) Inventors: Rajeev Samant, Mobile, AL (US); Lalita Samant, Mobile, AL (US)

(21) Appl. No.: 14/661,634

(22) Filed: Mar. 18, 2015

Related U.S. Application Data
(63) Continuation of application No. 13/254,811, filed on Nov. 15, 2011, now abandoned.
(60) Provisional application No. 61/158,243, filed on Mar. 6, 2009.

(51) Int. Cl. C12Q 1/68

(52) U.S. Cl. CPC C12Q 1/6886; C12Q 2600/106; C12Q 2600/158

(57) ABSTRACT

The present invention relates to methods and compositions for diagnosing and treating cancer, such as breast cancer and skin cancer.

### CROSS-REFERENCE TO RELATED APPLICATIONS

This application is a continuation of U.S. application Ser. No. 13/254,811 filed on Nov. 15, 2011 which is the U.S. National Phase of International Application No. PCT/US2010/025686 entitled METHODS AND COMPOSITIONS FOR THE DIAGNOSIS, PROGNOSIS AND TREATMENT OF CANCER, filed Feb. 26, 2010 and published in English on Sep. 10, 2010 as WO 2010/101793, which claims priority to U.S. Provisional Application No. 61/158,243 entitled “Methods and compositions for the diagnosis, prognosis and treatment of cancer” and filed on Mar. 6, 2009, the disclosures of which are hereby incorporated herein by reference in their entireties for any purpose.

### STATEMENT REGARDING FEDERALLY SPONSORED R&D

This invention was made with government support under DOD Grant/Contract Number BCRP-W81XWH-07-1-0400. The government has certain rights in the invention.

### REFERENCE TO SEQUENCE LISTING

The present application is being filed along with a Sequence Listing in electronic format. The Sequence Listing is provided as a file entitled USA_{—}001 VPC.TXT, created Feb. 24, 2010, which is approximately 60 KB in size. The information in the electronic format of the Sequence Listing is incorporated herein by reference in its entirety.

### FIELD OF THE INVENTION

The present invention relates to methods and compositions for diagnosing and treating cancer, such as breast cancer and skin cancer, such as melanoma.

### BACKGROUND

Cancer is a significant health problem throughout the world. Although advances have been made in detection and therapy of cancer, no vaccine or other universally successful method for prevention and/or treatment is currently available. Current therapies, which are generally based on a combination of chemotherapy or surgery and radiation, continue to prove inadequate in many patients.

Breast cancer, for example, is a significant health problem for women in the United States and throughout the world. Although advances have been made in detection and treatment of the disease, breast cancer remains the second leading cause of cancer-related deaths in women, affecting more than 180,000 women in the United States each year. For women in North America, the life-time odds of getting breast cancer are now one in eight.

No vaccine or other universally successful method for the prevention or treatment of breast cancer is currently available. Management of the disease currently relies on a combination of early diagnosis (through routine breast screening procedures) and aggressive treatment, which may include one or more of a variety of treatments such as surgery, radiotherapy, chemotherapy and hormone therapy. The course of treatment for a particular breast cancer is often selected based on a variety of prognostic parameters, including an analysis of specific tumor markers. See, e.g., Porter-Jordan and Lippman, Breast Cancer 8:73-100 (1994). However, the use of established markers often leads to a result that is difficult to interpret, and the high mortality observed in breast cancer patients indicates that improvements are needed in the treatment, diagnosis and prevention of the disease.

In spite of considerable research into therapies for these and other cancers, breast cancer remains difficult to diagnose and treat effectively. Accordingly, there is a need in the art for improved methods for detecting and treating such cancers.

### SUMMARY

The present invention relates to methods, compositions, and kits for the diagnosis, prognosis, treatment and amelioration of particular disorders, such as cancer. It has been found that expression of MRJ (Hsp40/DNAJB6) is lost in advanced infiltrating ductal carcinoma. Ectopic expression of the large isoform of MRJ, MRJ(L), is functionally capable of retarding tumor growth. MRJ(L) causes morphological changes indicative of epithelial-like appearance. MRJ(L) decreases the expression of several mesenchymal markers such as N-cadherin, Twist, Slug, vimentin and up regulated epithelial marker, Keratin 18. However, most noticeable is the loss of β-catenin protein upon MRJ(L) expression. Differential-proteomic analysis of the secretome showed that MRJ(L) expression elevated DKK1. Increased DKK1 levels enhanced proteosome mediated degradation of β-catenin and reduced Wnt/β-catenin signaling as measured by the TCF/LEF reporter activity in MRJ(L) expressors. Furthermore silencing MRJ(L) from immortalized mammary epithelial cell line, MCF10A, led to decreased DKK1 and E-cadherin levels, with concomitant increase of β-catenin protein. Silencing DKK1 from the MRJ(L) overexpressing cancer lines led to increased malignant behavior as measured by foci formation, growth in soft agar and wound healing capacity. Overall, findings provided herein show that MRJ(L) maintains the epithelial phenotype and restricts malignant behavior of tumor cells by inhibiting Wnt/β-catenin pathway.

Some embodiments of the present invention include methods for evaluating the presence, stage, or metastatic potential of a cancer in a subject comprising measuring the expression level of a nucleic acid encoding MRJ(L) or the expression level of MRJ(L) protein in a sample obtained from the subject.

Such methods can further comprise comparing said expression level of said nucleic acid encoding MRJ(L) or the expression level of said MRJ(L) protein in said sample to the expression level of said nucleic acid encoding MRJ(L) or the expression level of said MRJ(L) protein in normal tissue, tissue from a known stage of cancer, or cancerous tissue with a known metastatic potential.

In some methods, the sample comprises a nucleic acid sample removed from said subject's body, and the expression level of a nucleic acid encoding MRJ(L) is measured outside said subject's body. In more methods, the sample comprises a protein sample removed from said subject's body, and the level of MRJ(L) is measured outside said subject's body.

In some embodiments, a decreased level of expression of said MRJ(L) protein or said nucleic acid encoding said MRJ(L) protein indicates the presence, stage, or metastatic potential of a cancer.

Some embodiments further comprise measuring the level of expression of at least one marker in addition to MRJ(L).

In some embodiments, said measuring comprises measuring the level of mRNA encoding MRJ(L). In some embodiments, said measuring comprises measuring the level of said MRJ(L) protein.

In some embodiments, said at least one marker in addition to MRJ(L) comprises DKK-1. In some embodiments, a decrease in the level of expression of said at least one marker in addition to MRJ(L) indicates the presence, advanced stage, or significant metastatic potential of a cancer. In some embodiments, said at least one marker in addition to MRJ(L) comprises osteopontin, osteonectin, zinc binding α-2-glycoprotein, VGF nerve growth factor, nucleophosmin, N-cadherin, vimentin, twist and β-catenin. In some embodiments, an increase in the level of expression of said at least one marker in addition to MRJ(L) indicates the presence, advanced stage, or significant metastatic potential of a cancer.

In some embodiments, said cancer comprises skin cancer. In some embodiments, the cancer comprises a melanoma. In some embodiments, said cancer comprises breast cancer. In some embodiments, the cancer comprises an infiltrating ductal carcinoma.

In some embodiments, said sample comprises an epithelial cell. In some embodiments, said sample comprises a breast cancer cell. In some embodiments, said sample comprises a melanoma cell.

Some embodiments include pharmaceutical compositions. Such embodiments include a pharmaceutical composition comprising an agent that increases the level of MRJ(L) expression in a cell. In some embodiments, the composition comprises an isolated nucleic acid encoding MRJ(L) or fragment thereof. In some embodiments, the isolated nucleic acid is operably linked to a regulatory element. In some embodiments, the regulatory element comprises a promoter. In some embodiments, the agent comprises a polypeptide comprising the sequence of MJR(L), or fragment thereof.

Some embodiments include methods of treating a disorder. Such methods include treating a disorder comprising increasing the level of MRJ(L) in a cell of the subject. In some embodiments, the level of MRJ(L) is increased by administering an isolated nucleic acid encoding MRJ(L) or fragment thereof to said subject. In some embodiments, the disorder is selected from breast cancer, skin cancer, ovarian cancer, Huntington's disease, and cardiomyocyte hypertrophic growth. In some embodiments, the breast cancer comprises an infiltrating ductal carcinoma. In some embodiments, the skin cancer comprises melanoma.

Some embodiments include methods for screening for an agent for treating or preventing cancer. Such methods can include contacting a cell with a test agent; and selecting a test compound that increases the expression level of MRJ(L).

In some embodiments, the test agent decreases the expression level of at least one marker selected from osteopontin, osteonectin, zinc binding α-2-glycoprotein, VGF nerve growth factor, nucleophosmin N-cadherin, vimentin, twist and β-catenin. In some embodiments, the agent increases the expression level of DKK-1 and/or KiSS1. In some embodiments, the cell comprises an epithelial cell. In some embodiments, the cell comprises a breast cancer cell. In some embodiments, the cell comprises a skin cancer cell.

Some embodiments include an isolated antibody, or antigen binding fragment thereof, that specifically binds to human MRJ(L). In some embodiments, the antibody binds an epitope comprising amino acids 246-277 of the MRJ(L) polypeptide sequence, or a fragment thereof.

Some embodiments include kits for diagnosing cancer. In some embodiments, the kit can be used to diagnose a disorder selected from breast cancer, or skin cancer. Some kits can include a detection reagent that recognizes an mRNA encoding MRJ(L). Some embodiments further comprise a detection reagent that recognizes an mRNA encoding at least one marker selected from osteopontin, osteonectin, zinc binding α-2-glycoprotein, VGF nerve growth factor, nucleophosmin, N-cadherin, vimentin, twist, β-catenin, and DKK-1.

Some kits can include a detection reagent that binds MRJ(L) protein. In some embodiments, the reagent comprises an isolated antibody or fragment thereof that specifically binds MRJ(L) protein. Some embodiments further comprise a detection reagent that binds at least one marker selected from osteopontin, osteonectin, zinc binding α-2-glycoprotein, VGF nerve growth factor, nucleophosmin N-cadherin, vimentin, twist, β-catenin, DKK-1 and/or KiSS1.

In some embodiments, a kit can further comprise instructions for assessing the presence, stage, metastatic potential of said cancer based on the extent of binding to MRJ(L).

Some embodiments include nucleic acids encoding MRJ(L), or a fragment thereof.

More embodiments include methods for identifying an inducible nuclear localization sequence comprising: obtaining an isolated nucleic acid comprising a sequence encoding a polypeptide selected from SEQ ID NO:02, a fragment of SEQ ID NO:02, SEQ ID NO:04, and a fragment of SEQ ID NO:04; and providing said nucleic acid to a cell; stimulating said cell with a stimulus selected from heat shock, hypoxia, ILNα, IFNγ, and IL-1α; and evaluating the location of a polypeptide encoded by said nucleic acid. In some such methods the nucleic acid further comprises a reporter gene.

More embodiments include a polypeptide identified by the foregoing methods. Some such polypeptides can further comprise a therapeutic polypeptide. The therapeutic polypeptide can include hormones, growth factors, enzymes, receptors, antibodies, fragments of antibodies, drugs, oncogenes, tumor antigens, tumor suppressors, viral antigens, parasitic antigens, and bacterial antigens.

More embodiments include an isolated nucleic acid comprising a nucleic acid sequence encoding a polypeptide of SEQ ID NO:02, a fragment of SEQ ID NO:02, SEQ ID NO:04, and a fragment of SEQ ID NO:04, wherein the polypeptide comprises an inducible nuclear localization activity. Some embodiments include a nucleic acid further comprising a therapeutic nucleic acid. The therapeutic nucleic acid can encode a polypeptide selected from the group comprising hormones, growth factors, enzymes, receptors, antibodies, fragments of antibodies, drugs, oncogenes, tumor antigens, tumor suppressors, viral antigens, parasitic antigens, and bacterial antigens. In more embodiments, the therapeutic nucleic acid can include siRNAs, ribozymes, and other nucleic acids with therapeutic activity. In some embodiments, the inducible nuclear localization activity can be induced by a stimulus selected from heat shock, hypoxia, ILNα, IFNγ, and IL-1α. More embodiments include an isolated polypeptide encoded by the foregoing nucleic acids.

More embodiments include methods for inducing nuclear localization of an agent. Some such methods include stimulating a cell comprising an agent with a stimulus such as heat shock, hypoxia, ILNα, IFNγ, and IL-1α, wherein the agent comprises a polypeptide sequence such as SEQ ID NO:02, a fragment of SEQ ID NO:02, SEQ ID NO:04, and a fragment of SEQ ID NO:04, and wherein said polypeptide has an inducible nuclear localization activity. In some such embodiments the agent further comprises a therapeutic agent. The therapeutic agent can include hormones, growth factors, enzymes, receptors, antibodies, fragments of antibodies, drugs, oncogenes, tumor antigens, tumor suppressors, viral antigens, parasitic antigens, bacterial antigens, siRNAs, ribozymes, and nucleic acids having therapeutic activity.

Some embodiments include methods inducing a reversal of an epithelial to mesenchymal transition in a cell comprising: contacting said cell with an isolated polypeptide comprising a sequence selected from SEQ ID NO:02, a fragment of SEQ ID NO:02, SEQ ID NO:04, and a fragment of SEQ ID NO:04, wherein a reversal of an epithelial to mesenchymal transition in said cell is induced. Some such embodiments further comprise inducing said polypeptide to translocate to the nucleus of said cell. The inducing can include stimulating said cell with a stimulus selected from heat shock, hypoxia, ILNα, IFNγ, and IL-1α. In some embodiments, the cell is a cancer cell.

Some embodiments include methods for inducing a reversal of an epithelial to mesenchymal transition in a cell comprising: contacting said cell with an isolated nucleic acid comprising a sequence encoding a polypeptide selected from SEQ ID NO:02, a fragment of SEQ ID NO:02, SEQ ID NO:04, and a fragment of SEQ ID NO:04, wherein a reversal of an epithelial to mesenchymal transition in said cell is induced. Some such embodiments further comprise inducing said polypeptide to translocate to the nucleus of said cell. The inducing can include stimulating said cell with a stimulus selected from heat shock, hypoxia, ILNα, IFNγ, and IL-1α. In some embodiments, the cell is a cancer cell.

### BRIEF DESCRIPTION OF THE DRAWINGS

FIG. 1, panel (A) shows a graph of relative levels of MRJ(L) expressed in various breast cancer cell lines. FIG. 1, panel (B) shows a Western blot of MRJ isoforms expressed in various breast cancer cell lines. FIG. 1, panel (C) shows photomicrographs of tissues stained for MRJ, where Panel (C)1 shows a cystic hyperplasia and Panel (C)2 shows an infiltrating ductal carcinoma grade III.

FIG. 2, panel (A) shows a schematic comparing domains of the isoforms of DNAJB6 (mammalian relative of DnaJ; MRJ). Various domains are indicated with distinct colors. Domains common to both DNAJB6 isoforms bear the same color code. Domains specific to each isoform bear unique color code. The nuclear localization signal (NLS) sequence is represented using single letter code for the amino acids. The amino acids underlined and in bold represent the strongest NLS. FIG. 2, panel (B) shows a photomicrograph of COST cells transfected with pEGFP-N1-MRJ(L)^{WT} or pEGFP-N1-MRJ(L)^{mut}. Cells transfected with pEGFP-N1-MRJ(L)^{WT} show greater nuclear staining than cells transfected with pEGFP-N1-MRJ(L)^{mut}. FIG. 2, panel (C) shows a Western blot of nuclear extracts and cytoplasmic extracts.

FIG. 3, panel (A) shows a Western blot of MDJ(L) expression in MDA-MB-435 and MDA-MB-231 cells transfected with a MRJ(L) expression vector. FIG. 3, panel (B) shows photomicrographs and graphs of transfected cells in migratory assays. FIG. 3, panel (C) shows a graph of the number of colonies of transfected cells in an assay for anchorage dependence. FIG. 3, panel (D) shows a graph of the percentage of transfected cells that migrate through transwell in a migratory assay. FIG. 3, panel (E) shows a graph of the percentage of transfected cells that invade Matrigel-coated filters in an invasion assay.

FIG. 4, panel (A) shows a graph of tumor growth rate in mice implanted with MDA-MB-435 cells transfected with a MRJ(L) expression vector. FIG. 4, panel (B) shows a graph of tumor growth rate in mice implanted with MDA-MB-231 cells transfected with a MRJ(L) expression vector. FIG. 4, panel (C) shows a graph of the number of the percent number of metastases in a lung colonization assay with transfected MDA-MB-231 cells.

FIG. 5, panel (A) shows Western blots of proteins expressed in MDA-MB-435 and MDA-MB-231 cells transfected with a MRJ(L) expression vector. FIG. 5, panel (B) shows a graph of fold-change of gene expression in MDA-MB-435 transfected with a MRJ(L) expression vector in a quantitative real-time PCR assay.

FIG. 6 shows two Western blots of test bleed analysis from rabbit R3 shows specific recognition of MRJ(L), and no cross reactivity to MRJ(S). ΔJ=J domain deletion of MRJ(L).

FIG. 7 shows Western blot where test bleed from rabbit R3 specifically pulls down MRJ(L) and is incapable of precipitating MRJ(S).

FIG. 8, panel (A) shows a set of photomicrographs of change in cell morphology in stably transfected cells expressing MRJ(L). FIG. 8, panel (B) shows transcriptional analysis of stably transfected cells expressing MRJ(L) and controls.

FIG. 9, panel (A) shows a Western blot of DKK1 expression in stably transfected cells expressing MRJ(L) and controls. FIG. 9, panel (B) shows a graph of luciferase activity in stably transfected cells expressing MRJ(L) transfected with a reporter construct.

FIG. 10, panel (A) shows a transcriptional analysis of cells transfected with a β-catenin reporter construct. FIG. 10, panel (B) shows a graph of luciferase activity in stably transfected cells expressing MRJ(L), MRJ(S), or MRJ(L)ΔJ.

FIG. 11 shows photomicrographs of cells transfected with pEGFP-MRJ(L)^{MUT-NLS}, before and after treatment with heat shock. After heat shock, cell staining is concentrated in the nucleus compared to cell staining before heat shock.

FIG. 12A shows photomicrographs of cells transfected with pEGFP-MRJ(S), before and after treatment with heat shock. FIG. 12B shows photomicrographs of cells transfected with pEGFP-MR(S), before and after treatment with 5 hours hypoxia (2% O₂). Heat shock or hypoxia promoted nuclear localization of the expressed protein, MRJ(S)-EGFP from the cytoplasm

FIG. 13 shows photomicrographs of cells transfected with pEGFP-MRJ(S), after treatment with IFN-γ. Cell staining is localized to the nucleus.

FIG. 14 shows a graph of the relative expression of MRJ in stages of melanoma.

FIG. 15A shows photomicrograph of MDA-MB-435 cells transfected with constructs encoding MRJ(L) (435MRJ(L)), or control constructs (435V). FIG. 15B shows photomicrograph of MCF7 cells transfected with constructs encoding MRJ(L) (MCF7MRJ(L)), or control constructs (MCF7V). Scale—10 μm.

FIG. 16A shows a Western blot analysis for EMT markers expressed in MDA-MB-435 transfected with constructs encoding MRJ(L) (M), or control constructs (V). FIG. 16B shows a Western blot analysis for EMT markers expressed in MCF7 cells or MCF10CA1d.c1.1 transfected with constructs encoding MRJ(L) (M), or control constructs (V).

FIG. 17A shows a Western blot analysis of MDA-MB-435 cells transfected with constructs encoding MRJ(L) (435MRJ(L)), or control constructs (435V), and treated with vehicle (DMSO), MG132 or Lactacystin. FIG. 17B shows a Western blot analysis of MCF7 cells transfected with constructs encoding MRJ(L) (MCF7MRJ(L)), or control constructs (MCF7V), and treated with vehicle (DMSO), MG132 or Lactacystin.

FIG. 18, panel (A) shows a graph of percent luciferase activity from TCF/LEF reporter constructs in MDA-MB-435 cells transfected with constructs encoding MRJ(L) (435MRJ(L)), or control constructs (Vector). FIG. 18, panel (B) shows a graph of percent luciferase activity from TCF/LEF reporter constructs in MCF7 cells transfected with constructs encoding MRJ(L) (MCF7MRJ(L)), or control constructs (Vector). FIG. 18, panel (C) shows a graph of percent luciferase activity from TCF/LEF reporter constructs in MCF10CA1d.c1.1 cells transfected with constructs encoding MRJ(L) (MCF10CA1d.c1.1-MRJ(L)), or control constructs (Vector). Experiment were performed in triplicate and repeated twice. The error bars represent standard error of the mean. Statistical significance was determined if it reached 95% confidence. * indicates p<0.05.

FIG. 19, panel (A) shows a graph of fold-change in DKK1 transcript abundance in various breast cancer cell lines. Quantitative RT-PCR analysis was performed to measure DKK1 transcript level from different breast cancer cell lines as well as MDA-MB-435. Real-time quantitative RT-PCR analysis was performed on the experimental mRNAs in triplicate, and the experiment was repeated once from an independent passage. All the breast cancer cells showed significant downregulation compared to MCF10A (p<0.05). Significance was not calculated for MDA-MB-435. FIG. 19, panel (B) shows a Western blot analysis for secreted DKK-1 in MDA-MB-435 cells or MCF10CA1d.c1.1 cells transfected with constructs encoding MRJ(L) or control constructs (V). Cell lysates was immunoblotted and probed for MRJ(L). β-actin was used as a loading control for the cell lysate. The serum free-cell free-supernatant (SFM) was harvested from equal number of cells, concentrated to 10% of its original volume and immunoblotted for DKK1. FIG. 19, panel (C) shows graphs of percent luciferase activity of pDKK1-707 construct in MCF10CA1d.c1.1, MDA-MB-435, and MCF7 cells transfected with MRJ(L) or control constructs (Vector). The experiments were performed in triplicate and repeated twice. The error bars represent standard error of the mean. Statistical significance was determined if it reached 95% confidence. * indicates p<0.05. FIG. 19, panel (D) shows a graph of fold expression of DKK1 over vector in MCF7 or MDA-MB-435 cells

FIG. 20, panel (A) shows a Western blot analysis of MDA-MB-435 cells transfected with constructs encoding MRJ(L) or control constructs (V), and transiently transfected with siRNA designed to target DKK1 or non-targeting siRNA control. FIG. 20, panel (B) shows a Western blot analysis of MCF10CAc1.d cells transfected with constructs encoding MRJ(L) or control constructs (V), and transiently transfected with siRNA designed to target DKK1 or non-targeting siRNA control. FIG. 20, panel (C) shows a graph of fold change in transcript abundance for various markers in MDA-MB-435 cells transfected with constructs encoding MRJ(L) and transiently transfected with DKK-1 siRNA, over MDA-MB-435 cells transfected with constructs encoding MRJ(L) and transiently transfected with control.

FIG. 21, panel (A) shows a graph of number of colonies formed by MDA-MB-435 cells transfected with constructs encoding MRJ(L) and transiently transfected with siRNA designed to target DKK1 (siDKK1), or non-targeting siRNA (Control). FIG. 21, panel (B) shows a graph of percent motility of MDA-MB-435 cells transfected with constructs encoding MRJ(L) and transiently transfected with siRNA designed to target DKK1 (siDKK1), or non-targeting siRNA (Control) in a wound healing assay. FIG. 21, panel (C) shows a graph of number of colonies formed by MDA-MB-435 cells transfected with constructs encoding MRJ(L) and transiently transfected with siRNA designed to target DKK1 (siDKK1), or non-targeting siRNA (Control) in soft agar colonization assay. Experiments were performed in triplicate and repeated once. The error bars represent standard error of the mean. Statistical significance was determined if it reached 95% confidence. * indicates p<0.05.

FIG. 22, panel (A) shows a Western blot analysis of MCF10A cells transiently transfected with NM_{—}808 cloned in pSUPER.neo+GFP, or a scrambled control. Total protein (35 μg) was immunoblotted and analyzed for the levels of MRJ(L) and β-catenin β-tubulin was used to verify equal loading. FIG. 22, panel (B) shows a graph of luciferase activity in MCF10A cells transfected with the TCF/Lef reporter, TOPFlash, and transiently transfected with NM_{—}808, or a scrambled shRNA control. Experiments were performed in triplicate and repeated twice. The error bars represent standard error of the mean. Statistical significance was determined if it reached 95% confidence. * indicates p<0.05. FIG. 22, panel (C) shows a graph of fold change in transcript abundance for various markers in MCF10A cells transiently transfected with NM_{—}808, and compared to MCF10A cells transiently transfected with a scrambled shRNA control. Quantitative RT-PCR analysis was performed to measure transcript levels of epithelial markers E-cadherin and Keratin18. The knockdown of MRJ(L) and simultaneous downregulation of DKK1 was also monitored as a control. C-Myc levels were evaluated as an indicator of Wnt/β-catenin pathway activity. Analysis was performed on the experimental mRNAs in triplicate, and the experiment was repeated once. Statistical significance was determined if it reached 95% confidence. E-cadherin, MRJ(L) and DKK1 had significant changes compared to the scrambled shRNA control p<0.05.

FIG. 23 shows a graph of DKK1 and MRJ(L) expression in various cell lines.

### DETAILED DESCRIPTION

Numerous references are cited herein. The references cited herein are each to be considered incorporated by reference in their entirety into this specification. Embodiments of the present invention relates to methods and compositions for the diagnosis, prognosis and treatment of particular types of cancer, such as breast cancer and skin cancer.

MRJ (mammalian relative of DnaJ) is a class II DnaJ/Hsp40 family protein (Ohtsuka K, Hata M. Cell Stress Chaperones 2000, 5:98-112; and Seki N, et al., J Hum Genet 1999, 44:185-189). Members of the DnaJ/Hsp40 proteins are highly conserved and expressed in several tissues. They act as co-chaperones regulating protein folding, transport, translational initiation and gene expression. The Hsp40 family of proteins is known to have co-chaperonic activity. The role of Hsp40 family in a variety of aspects of tumor biology is being described only very recently (Mitra, A., et al. (2009) Clin Exp Metastasis; Tsai, M. F., et al. (2006) J Natl Cancer Inst 98, 825-838; Chen, H. W., et al. (2008) Cancer Res 68, 7428-7438; Wang, C. C., et al. (2007) Cancer Res 67, 4816-4826; and Cheng, H., et al. (2005) Mol Cell Biol 25, 44-59). DNAJB6 is a member of the class II Hsp40/DnaJ family of proteins comprising two differentially spliced variants (Mitra, A., et al. (2008) Breast Cancer Res 10, R22). MRJ(S), the shorter variant of DNAJB6, has been widely studied and has been reported to regulate keratin8/k18 organization, assist in NFATc3 mediated transcriptional repression and play a role in Huntington's disease (Dai, Y. et al. (2005) Mol Cell Biol 25, 9936-9948; Izawa, I., et al. (2000) J Biol Chem 275, 34521-34527; Chuang, J. Z., et al. (2002) J Biol Chem 277, 19831-19838).

In contrast to MRJ(S), the role of the long variant, MRJ(L), in various pathophysiologies has still to be revealed with the exception of a potential role in the nuclear import of HIV-2 vpx (Cheng, X., et al. (2008) J Virol 82, 1229-1237). Loss of DNAJB6 in advanced breast cancer patients has been observed. Moreover, MRJ(L) can act as a nuclear protein by virtue of its nuclear localization signal (NLS) and can considerably reduce the expression of several secreted proteins that promote invasion, migration and metastasis of cancer cells. Functionally, MRJ(L) can reduce tumorigenicity and metastasis of melanoma and breast cancer cells.

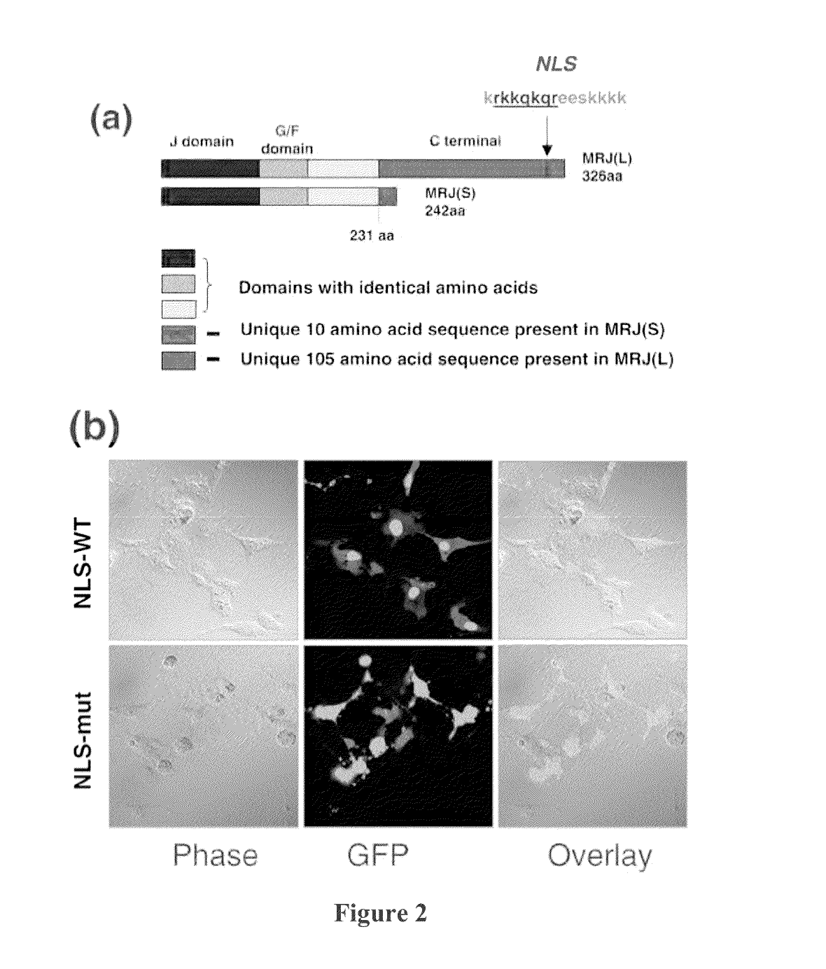

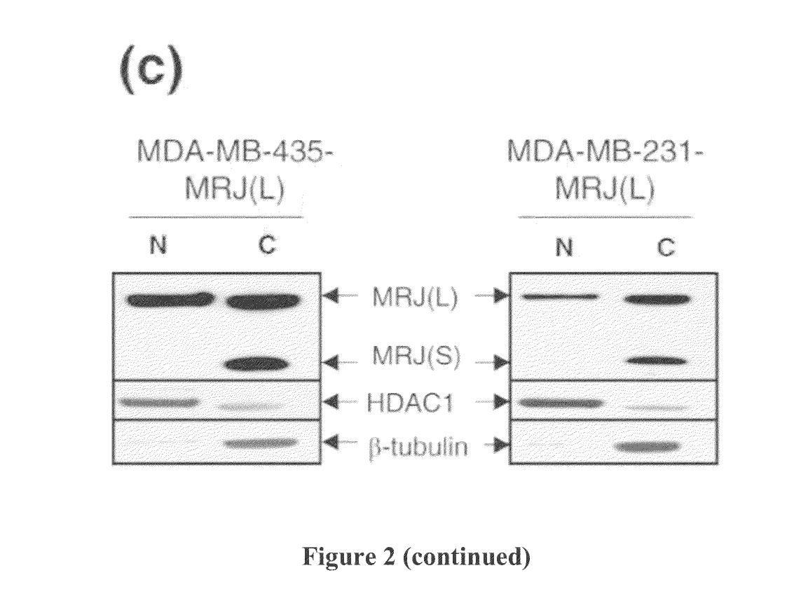

In humans, the MRJ gene maps to chromosome 7q36.3 and encodes two splice variants, namely, MRJ(L) (isoform a) and MRJ(S) (isoform b). The full length DNAJB6 isoform, MRJ(L) comprises 326 amino acids (NCBI Accession Numbers mRNA: GI:34328906; Polypeptide: GI:17388799). The shorter isoform, MRJ(S), comprises 242 amino acids (NCBI Accession Numbers mRNA: GI:24234719. Polypeptide: GI:4885495). MRJ(S) lacks the C-terminal 95 amino acids of MRJ(L) but contains an additional 10 amino acids (KEQLLRLDNK) (SEQ ID NO:27). MRJ(L) has a predicted NLS located in its C-terminal region, which is absent in MRJ(S). Besides the differences at the C-terminus, both isoforms share identical structure containing a conserved J domain (70 aa) and a glycine/phenylalanine domain, namely a G/F domain (FIG. 2, panel (A)).

Previous studies have focused on MRJ(S). For example, a role as a molecular chaperone has been implicated for MRJ(S) in Huntington's disease, where overexpression MRJ(S) effectively suppressed polyglutamine-dependent protein aggregation, caspase activity, and cellular toxicity in neurons (Chuang J Z, et al. J Biol Chem 2002, 277:19831-19838; and Fayazi Z, et al. Neurobiol Dis 2006, 24:226-244.).). In addition, MRJ(S) has also been shown to regulate keratin 8/18 filament organization (Izawa I, et al. J Biol Chem 2000, 275:34521-34527.). MRJ(S) may also have a role in blocking calcineurin-induced cardiomyocyte hypertrophy (Dai Y S, et al. Mol Cell Biol 2005, 25:9936-9948).

MRJ(L) is likely to have a role as a negative regulator in tumor growth. For example, MRJ(L) is expressed in aggressive cancer cell lines, including advanced grade infiltrating ductal carcinoma, at a significantly lower level than in normal tissues, such as breast. This is in contrast to MRJ(S) which was present in all cancer cell lines tested. Surprisingly, stable transfection of MRJ(L) constitutive expression vectors into cancer cell lines produces dramatic changes associated with malignancy. In a series of experiments, the highly aggressive cancer cell lines, MDA-MB-231 and MDA-MB-435 were stably transfected with MRJ(L) constitutive expression vectors (Mitra et al., “Large isoform of MRJ (DNAJB6) reduces malignant activity of breast cancer.” Breast Cancer Res. 10:R22, incorporated by reference in its entirety). MDA-MB-231 is a breast cancer cell-line. While the MDA-MB-435 cell line was initially thought to have characteristics of a breast cancer cell line, it may be more characterized as a melanoma cell line. In a set of in vitro experiments, the MRJ(L) expressing MDA-MB-231 and MDA-MB-435 cells showed reduced wound healing, invasion, migration and anchorage-independent growth in assays. These observations indicate a role for MRJ(L) in reducing tumor progression and metastasis. In another set of experiments, MRJ(L) expressing MDA-MB-231 and MDA-MB-435 cells were independently assayed for orthotopic tumor growth in nude mice. Both cell lines showed retarded tumor growth compared to cells transfected with vector only. In another set of experiments, MRJ(L) expressing MDA-MB-231 cells showed a significantly decreased ability to establish pulmonary metastases upon tail vein injection into athymic mice, compared to mice injected with MDA-MB-231 cells transfected with vector only. These experiments show that MRJ(L) may be used to diagnose types of cancer, such as breast cancer and/or skin cancer, and may be used to downregulate tumor growth and metastasis.

MRJ(L) may function by modulating expression of members of the Wnt/β-catenin signaling pathway, for example, Dickkopf-1 (DKK-1) (NCBI Accession Number GI:61676924). DKK-1 is an extracellular antagonist of the Wnt/β-catenin signaling pathway. Extracellular antagonists of the Wnt/β-catenin signaling pathway include two broad classes and can prevent ligand-receptor interactions by various mechanisms. The first class comprises members of the Dickkopf (Dkk) family, such as DKK-1, which bind to one subunit of the Wnt receptor complex, namely, LRP 5/6. A second class includes members such as the secreted Frizzled-related protein (sFRP) family, Wnt inhibitory factor-1 (WIF-1), and Cerberus.

Activation of the Wnt/β-catenin pathway includes binding of Wnt proteins to cell-surface receptors of the Frizzled family and activation of the Dishevelled family of proteins, such as Dishevelled (DSH). DSH is a component of a membrane-associated Wnt receptor complex which, when activated by Wnt binding, inhibits a second complex of proteins that includes axin, GSK-3, and APC. The axin/GSK-3/APC complex normally promotes the proteolytic degradation of the β-catenin intracellular signaling molecule. However, when the “β-catenin destruction complex” is inhibited, a pool of cytoplasmic β-catenin stabilizes, and β-catenin is able to enter the nucleus and interact with the TCF/LEF family transcription factors to promote specific gene expression.

In experiments with MRJ(L) expressing MDA-MB-435 cells, levels of DKK-1 mRNA were 10-fold greater than vector-only controls. Moreover, in experiments with MRJ(L) expressing MDA-MB-435 cells transfected with reporter constructs containing the DKK-1 promoter, a 10-fold increase in activity was also observed compared to vector only controls. These experiments indicate that levels of DKK-1 protein, and the DKK-1 promoter are responsive to MRJ(L).

In more experiments with MRJ(L) expressing MDA-MB-435 cells, levels of β-catenin were inhibited compared to vector-only controls. These experiments show that MRJ(L) reduces the level of β-catenin. In additional experiments, MDA-MB-435 cells were stably transfected with constructs encoding a variant of the MRJ(L) protein without a J-domain (MRJΔJ). In a set of transient transfection experiments with a reporter construct to measure β-catenin-mediated transcription, MRJ(L) expressing MDA-MB-435 cells inhibited transcription from the reporter construct. However, MDA-MB-435 cells containing MRJΔJ showed no significant inhibition of transcription from the reporter construct. Similarly, MDA-MB-435 cells containing MRJ(S) showed no significant inhibition of transcription from the reporter construct due to the lack of nuclear localization. These experiments indicate that MJR(L) can inhibit β-catenin-mediated transcription, and the J-domain of MJR(L) is involved in the suppression of the β-catenin-mediated transcriptional activation.

Activation of DKK-1 can cause apoptosis and reduced tumor growth in nude mice (Mikheev A. M. et al., “Dickkopf-1 mediated tumor suppression in human breast carcinoma cells” Breast cancer Res Treat 112:263-73 (2008)). Recent studies on the mechanism of myc-induced mammary epithelial cell transformation showed that c-Myc suppresses the Wnt inhibitors DKK-1 and sFRP1, and derepression of DKK-1 or sFRP1 reduces Myc-dependent transforming activity. DKK-1 was also shown to inhibit the transformed phenotype of breast cancer cell lines, and inhibits tumor formation (Cowling V. H. et al., “Turning the Tables: Myc Activates Wnt in Breast Cancer” Cell Cycle 6:2625-7 (2007); “c-Myc transforms human mammary epithelial cells through repression of the Wnt inhibitors DKK1 and SFRP1” Mol. Cell. Biol. 27:5135-46(2007)).

Other proteins that may have roles in promoting cancer progression are downregulated in MDA-MB-435 cells stably transfected with MRJ(L) constitutive expression vectors. For example, osteopontin, osteonectin, nucleophosmin, VGF nerve growth factor, and zinc binding α-2-glycoprotein 1.

Osteopontin is an RGD binding glycoprotein which plays a prominent part in steps of breast tumor growth and metastasis (Rittling S. R. et al., “Role of osteopontin in tumour progression” Br. J. Cancer 90:1877-81). Osteonectin has also been shown to regulate adhesion and spreading of various types of tumor cells (Ledda M. F. et al., “Suppression of SPARC expression by antisense RNA abrogates the tumorigenicity of human melanoma cells” Nat Med 3:171-6 (1997)). Antisense oligonucleotides directed against osteopontin and osteonectin have been shown to inhibit proliferation and migration of MDA-MB-231 cells (Adwan et al., “Downregulation of osteopontin and bone sialoprotein II is related to reduced colony formation and metastasis formation of MDA-MB-231 human breast cancer cells” Cancer Gene Ther. 11:109-20). Also, osteonectin has been reported to play a role in migration of breast cancer cells to bone (Campo McKnight D. A. et al., “Roles of osteonectin in the migration of breast cancer cells into bone.” J Cell Biochem 2006, 97:288-302). However there are conflicting reports about the exact role of osteonectin in breast cancer. A study by Koblinski et al. reports that SPARC expression inhibits MDA-MB-231 metastasis (Koblinski et al., “Endogenous osteonectin/SPARC/BM-40 expression inhibits MDA-MB-231 breast cancer cell metastasis.” Cancer Res 2005, 65:7370-7377). However the primary tumor growth rates (in vivo growth) of SPARC expressing MDA-MB-231 were not studied.

Zinc binding α-2-glycoprotein 1 (AZGP1) has been reported to be a potential quantitative marker of differentiation grade of oral tumors (Brysk M. M. et al., “Zinc-alpha2-glycoprotein expression as a marker of differentiation in human oral tumors.” Cancer Lett 1999, 137:117-120). It is expressed by malignant prostatic epithelium and may serve as a potential serum marker for prostate cancer. The levels of AZGP1 have been found to be higher in well differentiated than in moderate or poorly differentiated breast tumors. Relevance of VGF nerve growth factor in breast cancer has not yet been explored (Diez-Itza I., et al., “Zn-alpha 2-glycoprotein levels in breast cancer cytosols and correlation with clinical, histological and biochemical parameters.” Eur J Cancer 1993, 29A:1256-1260). However, peptide products of the neurotrophin-inducible gene VGF are produced in human neuroendocrine cells from early development and increase in hyperplasia and neoplasia (Rindi G. et al., “Peptide products of the neurotrophin-inducible gene vgf are produced in human neuroendocrine cells from early development and increase in hyperplasia and neoplasia.” J Clin Endocrinol Metab 2007, 92:2811-2815).

Nucleophosmin, NPM1, is a 37 kd phosphoprotein with nucleo-cytoplasmic shuttling properties present in higher levels in proliferating cancer cells compared to quiescent cells and its increased expression in hepatocellular carcinoma correlates with clinicopathological parameters (Yun J.P. et al., “Increased expression of nucleophosmin/B23 in hepatocellular carcinoma and correlation with clinicopathological parameters.” Br J Cancer 2007, 96:477-484).

The expression of KiSS1 (metastin) has been reported to have an inverse correlation with human tumor progression and metastasis and is either reduced or absent in various types of cancers (Sanchez-carbayo M. et al., “Tumor suppressor role of KiSS-1 in bladder cancer: loss of KiSS-1 expression is associated with bladder cancer progression and clinical outcome.” Am J Pathol 2003, 162:609-617.). Overexpression of KiSS1 or treatment of metastatic breast cancer cells with synthetic KiSS1 have been shown to reduce their metastatic potential (Muir A I, et al., “AXOR12, a novel human G protein-coupled receptor, activated by the peptide KiSS-1.” J Biol Chem 2001, 276:28969-28975; Ohtaki T, et al. “Metastasis suppressor gene KiSS-1 encodes peptide ligand of a G-protein-coupled receptor.” Nature 2001, 411:613-617.). Thus the secreted proteome analysis revealed molecular players downstream of MRJ(L) and that the changes in the secreted proteome undoubtedly indicated a change from aggressive to non-aggressive phenotype. MRJ(L) expression upregulated KiSS1 and reduced the metastatic ability of the cells.

Table 1 provides examples of sequences that may be used in the methods and compositions provided herein.

TABLE 1

| | mRNA | Polypeptide |
|---|---|---|
| human MRJ(L) | (SEQ ID NO: 01) | (SEQ ID NO: 02) |
| human MRJ(S) | (SEQ ID NO: 03) | (SEQ ID NO: 04) |
| human DKK-1 | (SEQ ID NO: 05) | (SEQ ID NO: 06) |
| human VGF | (SEQ ID NO: 07) | (SEQ ID NO: 08) |
| human AZGP1 | (SEQ ID NO: 09) | (SEQ ID NO: 10) |
| human SPARC | (SEQ ID NO: 11) | (SEQ ID NO: 12) |
| human KiSS1 | (SEQ ID NO: 13) | (SEQ ID NO: 14) |
| human SPP1 (isoform a) | (SEQ ID NO: 15) | (SEQ ID NO: 16) |
| human SPP1 (isoform b) | (SEQ ID NO: 17) | (SEQ ID NO: 18) |
| human SPP1 (isoform c) | (SEQ ID NO: 19) | (SEQ ID NO: 20) |
| human NPM-1 (isoform 1) | (SEQ ID NO: 21) | (SEQ ID NO: 22) |
| human NPM-1 (isoform 2) | (SEQ ID NO: 23) | (SEQ ID NO: 24) |
| human NPM-1 (isoform 3) | (SEQ ID NO: 25) | (SEQ ID NO: 26) |

### Inducible Nuclear Localization Compositions and Methods

The nuclear localization of DNAJB6 is involved in its activity. The small spliced variant of DNAJB6, MRJ(S), is exclusively localized to cytoplasm, however it can enter nucleus upon certain stimuli such as heat shock, IFN-γ, hypoxia, and the like. Once in nucleus, MRJ(S) can down regulate Wnt/β-catenin signaling. Downregulation of β-catenin dependent transcription leads to down regulation of transcription factors Slug and Twist. These transcription factors regulate proteins that contribute to a mesenchymal phenotype.

The MRJ(S) pre-proprotein contains no classical nuclear localization signal, and is predominantly located in the cytoplasm of cells. However, protein reporter constructs show that the protein translocates from the cytoplasm to the nucleus in cells that have been treated with hypoxia (2% O₂), heat shock (42° C.), IFN-α, IFN-γ, and IL-1α. It is envisioned that MRJ(S) sequences involved in the response to nuclear translocating agents such as hypoxia (2% O₂), heat shock (42° C.), IFN-α, IFN-γ, and IL-1α are mapped. In some embodiments, MRJ(S) sequences that are responsive to nuclear translocating agents can be linked to other proteins to form hybrid proteins. Such hybrid proteins can be used to deliver protein sequences to the nucleus in response to particular stimulation, for example, treatment with hypoxia (2% O₂), heat shock (42° C.), IFN-α, IFN-γ, and IL-1α.

Techniques that can be used to map regions of biological activity are well known in the art. For example, deletion analysis can be performed on MRJ(S) sequences where various mutant MRJ(S) polypeptides with specific deletions can be linked to a reporter polypeptide, such a EGFP. Mutant MRJ(S)-EGFP hybrids encoding polypeptides can be transfected into cells, such as COST cells, and the ability of the mutant MRJ(S)-EGFP hybrid polypeptides to translocate to the nucleus in response to nuclear translocating agents can be assessed. Similarly, mutagenesis analyses can be used to further map sequences within the MRJ(S) polypeptide to determine specific sequences necessary for translocating the MRJ(S) protein to the nucleus.

Some embodiments include methods for identifying inducible nuclear localization sequences. Such sequences include polypeptide sequences that may be induced to be transported to the nucleus of a cell. For example, polypeptides can include MRJ(L) and MRJ(S), and fragments thereof. Examples of polypeptides encoding MRJ(L) and (MRJ(S) include SEQ ID NO:02 and SEQ ID NO:04, respectively. In some embodiments, the fragment may contain at least about 5, at least about 10, at least about 15, at least about 20, at least about 25, at least about 30, at least about 35, at least about 40, at least about 45, at least about 50 consecutive amino acids of the polypeptides of SEQ ID NO:02, or SEQ ID NO:04, namely, MRJ(L), or MRJ(S), respectively. In more embodiments, sequences with inducible nuclear localization activity can include nucleic acids encoding MRJ(L) or MRJ(S), and fragments thereof. Examples of nucleic acids encoding MRJ(L) and MRJ(S) include SEQ ID NO:01 and SEQ ID NO:03, respectively. In some embodiments, the fragment may contain at least about 5, at least about 10, at least about 15, at least about 20, at least about 25, at least about 30, at least about 35, at least about 40, at least about 45, at least about 50 consecutive nucleotides of the nucleic acids of SEQ ID NO:01, or SEQ ID NO:03, namely, nucleic encoding MRJ(L), or MRJ(S), respectively.

Some methods to identify inducible nuclear localization sequences can include obtaining an isolated nucleic acid comprising a sequence encoding a polypeptide selected from SEQ ID NO:02, a fragment of SEQ ID NO:02, SEQ ID NO:04, and a fragment of SEQ ID NO:04, and providing the nucleic acid to a cell. The cell can be stimulated to induce nuclear localization of the a sequence encoding a polypeptide with inducible nuclear localization activity. Examples of stimuli include heat shock, hypoxia, ILNα, IFNγ, and IL-1α. More embodiments include evaluating the location of a polypeptide encoded by the nucleic acid, whereby translocation of the polypeptide to the nucleus on stimulation can identify a polypeptide sequence having inducible nuclear localization activity.

Nucleic acids encoding polypeptides with inducible nuclear localization activity such as nucleic acids comprising SEQ ID NO:01, SEQ ID NO:03, or fragments thereof, or nucleic acids encoding polypeptides comprising SEQ ID NO:02, SEQ ID NO:04, or fragments thereof, can be operably linked to other sequences. Other sequences can include therapeutic nucleic acids, reporter genes and the like. In some embodiments, therapeutic nucleic acids can encode polypeptides with an increased activity on translocation to the nucleus. For example, therapeutic nucleic acids encoding nuclear receptors may have enhanced activity in the nucleus. In some embodiments, a polypeptide encoded by a therapeutic nucleic acid may be activated during translocation to the nucleus, before translocation to the nucleus, or after translocation to the nucleus. Activation may include a conformation change in the polypeptide encoded by the therapeutic nucleic acid, and/or cleavage of inhibitory sequences.

Examples of therapeutic nucleic acids can include nucleic acids encoding receptors, enzymes, ligands, regulatory factors, hormones, antibodies or antibody fragments and structural proteins. Therapeutic nucleic acid sequences also include sequences encoding nuclear proteins, cytoplasmic proteins, mitochondrial proteins, secreted proteins, membrane-associated proteins, serum proteins, viral antigens, bacterial antigens, protozoal antigens and parasitic antigens. More examples include nucleic acids encoding hormones, growth factors, enzymes, receptors, antibodies, fragments of antibodies, polypeptide drugs, oncogenes, tumor antigens, tumor suppressors. Specific examples include nucleic acids encoding growth hormone, androgen receptors, oncoproteins (for example, those encoded by ras, fos, myc, erb, src, neu, sis, jun), HPV E6 or E7 oncoproteins, p53 protein, Rb protein, cytokine receptors, and proteins from viral, bacterial and parasitic organisms and other proteins of useful significance in the body. In some embodiments, the therapeutic nucleic acid may be siRNA, ribozyme, or other nucleic acid having a therapeutic activity. The choice of gene, to be incorporated, is only limited by the availability of the nucleic acid sequence encoding it. One skilled in the art will readily recognize that as more proteins and polypeptides become identified they can be integrated into the polynucleotide of the present invention and expressed.

More embodiments include polypeptides with inducible nuclear localization sequence activity linked to therapeutic agents. The polypeptides with inducible nuclear localization activity include polypeptides comprising SEQ ID NO:02, SEQ ID NO:04, and fragments thereof. Therapeutic agents can include polypeptides encoded by therapeutic nucleic acids. Additionally, therapeutic agents can include non-polypeptide compositions, for example chemical compounds, such as compounds with pharmaceutical activity, as well as nucleic acids such as siRNAs, ribozymes, and other nucleic acids having a therapeutic activity. Non-polypeptide agents can be linked to polypeptides with inducible nuclear localization sequence activity linked to therapeutic agents using techniques well known in the art.

More embodiments include methods for inducing nuclear localization of an agent in a cell. Such methods can include stimulating a cell with a stimulus such as heat shock, hypoxia, ILNα, IFNγ, and IL-1α. The agent can include a polypeptide having inducible nuclear localization. In some embodiments, the agent can also include a therapeutic agent.

More embodiments include methods for high throughput screening to determine stimuli that can induce nuclear localization of a polypeptide comprising SEQ ID NO:02, SEQ ID NO:04, namely, MRJ(L), or MRJ(S), respectively, and fragments thereof, or nucleic acids encoding SEQ ID NO:01, SEQ ID NO:02, namely, nucleic acids encoding MRJ(L), or MRJ(S), respectively, and fragments thereof. Examples of stimuli can include physical conditions such as changes in temperature, and changes in physical conditions, such as oxygen concentration. More examples of stimuli include biological compounds such as ILNα, IFNγ, and IL-1α. More examples of stimuli include chemical compounds, such as small molecules. Such methods can include obtaining a cell comprising a polypeptide with inducible nuclear localization activity comprising SEQ ID NO:02, SEQ ID NO:04, namely, MRJ(L), or MRJ(S), respectively, and fragments thereof, or nucleic acids encoding SEQ ID NO:01, SEQ ID NO:02, namely, nucleic acids encoding MRJ(L), or MRJ(S), respectively, and fragments thereof. In some embodiments, the polypeptide can further comprise a reporter moiety. For example, the reporter moiety can comprise E-GFP. In some embodiments, the cell can be contacted or exposed to a putative stimulus for inducing nuclear translocation of the polypeptide. Translocation of the polypeptide to the nucleus of the cell can identify a stimulus to induce nuclear localization of the polypeptide. For example, on contacting a cell comprising a polypeptide comprising inducible nuclear localization activity comprising SEQ ID NO:02, SEQ ID NO:04, namely, MRJ(L), or MRJ(S), respectively, and fragments thereof, or nucleic acids encoding SEQ ID NO:01, SEQ ID NO:02, namely, nucleic acids encoding MRJ(L), or MRJ(S), respectively, and fragments thereof, and a reporter polypeptide, such as E-GFP, with a putative stimulus, the polypeptide translocates to the nucleus of the cell. The translocation is identified by imaging the cell. The putative stimulus is identified.

### Methods of Diagnosis

Some embodiments relate to methods for diagnosis and prognosis of particular types of cancer, such as breast, skin, melanoma, ovarian, pancreatic, lung cancer. In some embodiments, the stage of cancer or metastatic potential of a cancer is assessed by measuring the amount of a nucleic acid encoding MRJ(L) or the amount of MRJ(L) protein in a biological sample. In some aspects of such embodiments, the amount of nucleic acid encoding MRJ(L) or the amount of MRJ(L) protein in a biological sample is compared to that of a control sample indicative of non-cancerous tissues, a particular stage of cancer, or cancer with metastatic potential. Alternatively, the amount of nucleic acid encoding MRJ(L) or the amount of MRJ(L) protein in a biological sample may be compared to the amount of nucleic acid encoding MRJ(L) or the amount of MRJ(L) protein known to be indicative of cancer, a particular stage of cancer, or to an amount known to be indicative of non-cancerous tissue. Some embodiments include removing a sample from a subject's body. For example, measuring and comparing can be ex vivo.

In some embodiments, the level of a nucleic acid encoding a marker in addition to a nucleic acid encoding MRJ(L) or the level of a protein marker in addition to MRJ(L) is measured. For example, the additional marker may be MRJ(S), DKK-1, osteopontin, osteonectin, zinc binding α-2-glycoprotein, VGF nerve growth factor, nucleophosmin N-cadherin, vimentin, twist, KiSS1 or β-catenin. A biological sample can be any sample suitable for measuring the level of a nucleic acid encoding MRJ(L), or for measuring MRJ(L) protein. For example, the biological sample can include blood, sera, sputum urine and tumor biopsies, including epithelial cells and breast cancer cells obtained from a patient.

Expression levels can be measured by various methods, such as levels of mRNA, levels of protein, and levels of biological activity of a protein or mRNA. Typically, the increase or decrease in expression of a marker is relative to a non-cancerous control.

Polynucleotide primers and probes may be used to detect the level of mRNA encoding MRJ(L) or an additional marker protein, which is also indicative of the presence or absence of a cancer. In general, a marker sequence may be present at a level that is increased or decreased at least two-fold, preferably three-fold, and more in tumor tissue than in normal tissue of the same type from which the tumor arose. Expression levels of a particular marker sequence in tissue types different from that in which the tumor arose are irrelevant in certain diagnostic embodiments since the presence of tumor cells can be confirmed by observation of predetermined differential expression levels, e.g., about 2-fold, 5-fold, etc, in tumor tissue to expression levels in normal tissue of the same type.

In some embodiments, a decrease in the levels of expression of a marker or nucleic acid encoding a marker, such as MRJ(L), MRJ(S), DKK-1 and KiSS1, in a sample relative to expression levels in normal tissue, tissue from less advanced stages of cancer, or cancer with metastatic potential, can indicate the stage or metastatic potential of a cancer. In such embodiments, the decrease can be about 2-fold, 5-fold, 10-fold, 100-fold, or more. In some embodiments, an increase in the level of expression in a sample of a marker such as osteopontin, osteonectin, zinc binding α-2-glycoprotein, VGF nerve growth factor, nucleophosmin N-cadherin, vimentin, and twist, relative to expression levels in normal tissue, tissue from less advanced stages of cancer, or cancer with metastatic potential, can indicate the stage or metastatic potential of a cancer. In such embodiments, the increase can be about 2-fold, 5-fold, 10-fold, 100-fold, or more.

In certain embodiments, the presence, stage, or metastatic potential of cancer can be assessed by comparing the level of expression of at least one marker in a biological sample and non-cancerous control sample. Such embodiments include measuring the level of expression of MRJ(L) protein or nucleic acid encoding MRJ(L). More embodiments can include measuring the level of expression of MRJ(L) and the level of expression of an additional marker.

Differential expression patterns can be utilized advantageously for diagnostic purposes. For example, in one aspect described herein, altered expression levels of MRJ(L) and, in some embodiments, an additional marker in tumor tissue relative to normal tissue of the same type, but not in other normal tissue types can be exploited diagnostically. For example, the presence of metastatic tumor cells, such as in a sample taken from the circulation or some other tissue site different from that in which the tumor arose, can be identified and/or confirmed by detecting altered expression of MRJ(L), and in some embodiments, an additional marker in the sample, for example using RT-PCR analysis or other methodologies for measuring nucleic acid levels. In many instances, it will be desired to enrich for tumor cells in the sample of interest using cell capture or other like techniques.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a subject may be determined by (a) contacting a biological sample obtained from a subject with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. In such embodiments, the binding agent can comprise an antibody or fragment thereof specific to MRJ(L). Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length breast tumor proteins and polypeptide portions thereof to which the binding agent binds, for example the MRJ(L) protein or additional markers described herein.

The solid support may be any material known to those of ordinary skill in the art to which the binding agent may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 μg to about 10 μg, and preferably about 100 μg to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art may be used, such as bovine serum albumin or TWEEN 20. (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with breast cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% TWEEN 20. The second antibody, which contains a reporter group, may then be added to the solid support. Reporter groups are well known in the art.

The detection reagent is then incubated with the immobilized antibody-polypeptide complex for an amount of time sufficient to detect the bound detection reagent. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as breast cancer, the signal detected from the reporter group that remains bound to the solid support is generally compared to a signal that corresponds to a predetermined cut-off value. In one embodiment, the cut-off value for the detection of a cancer is the average mean signal obtained when the immobilized antibody is incubated with samples from patients without the cancer. In general, a sample generating a signal that is three standard deviations above or below the predetermined cut-off value is considered positive for the cancer. For example, a reduced level of MRJ(L) protein or an additional marker downregulated by MRJ(L) may be indicative of the presence of cancer, the stage of cancer, or the metastatic potential of cancer. Similarly, a reduced level of MRJ(L) protein or an additional marker upregulated by MRJ(L) may be indicative of the presence of cancer, the stage of cancer, or the metastatic potential of cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer. It will be understood that such embodiments can be applied where a decrease in the level of expression of a marker is used to detect cancer, or indicate progression of cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described herein. In the strip test format, one end of the membrane to which binding agent is bound is immersed in a solution containing the sample. The sample migrates along the membrane through a region containing second binding agent and to the area of immobilized binding agent. The amount of immobilized antibody indicates the presence, stage, or metastatic potential of a cancer. Typically, the concentration of second binding agent at that site generates a pattern, such as a line, that can be read visually. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 μg to about 1 μg, and more preferably from about 50 μg to about 500 μg. Such tests can typically be performed with a very small amount of biological sample.

Of course, numerous other assay protocols exist that are suitable for use with the markers described herein. The above descriptions are intended to be examples only. It will be apparent to those of ordinary skill in the art that the above protocols may be readily modified to use marker polypeptides to detect antibodies that bind to such polypeptides in a biological sample. The detection of such marker-specific antibodies may correlate with the presence of a cancer.

As noted herein, a cancer, the stage of cancer, or metastatic potential of cancer, may also, or alternatively, be detected based on the level of mRNA encoding MRJ(L) and, in some embodiments, an additional marker in a biological sample. For example, at least two oligonucleotide primers may be employed in a polymerase chain reaction (PCR) based assay to amplify a portion of a marker cDNA derived from a biological sample, wherein at least one of the oligonucleotide primers is specific for a polynucleotide encoding the marker. The amplified cDNA is then separated and detected using techniques well known in the art, such as gel electrophoresis. Similarly, oligonucleotide probes that specifically hybridize to a polynucleotide encoding a tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a marker described herein that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989). In some embodiments, the primers and probes may hybridize to MRJ(L) nucleic acids, but not to MRJ(S) nucleic acids.

One embodiment employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater change in expression in several dilutions of the test patient sample as compared to the same dilutions of the non-cancerous sample may typically considered positive.

In some embodiments, the methods and compositions described herein may be used to identify the progression of cancer. In such embodiments, assays as described herein for the diagnosis of a cancer may be performed over time, and the change in the level of reactive polypeptide(s) or polynucleotide(s) evaluated. For example, the assays may be performed every month for a period of 6 months to 1 year, and thereafter performed as needed. In general, a cancer is progressing in those patients in whom the level of polypeptide or polynucleotide detected changes over time. For example, a cancer, such as breast cancer may be progressing where levels of expression of markers such as MRJ(L), DKK-1, and KiSS1 are decreasing, and/or levels of expression of markers such as osteopontin, osteonectin, zinc binding α-2-glycoprotein, VGF nerve growth factor, nucleophosmin N-cadherin, vimentin, and twist are increasing. In some embodiments, the level of expression of a marker can be used to determine the progression of a cancer.

Certain in vivo diagnostic assays may be performed directly on a tumor. One such assay involves contacting tumor cells with a binding agent, for example, an isolated antibody or fragment thereof, specific for MRJ(L). The bound binding agent may then be detected directly or indirectly via a reporter group. Such binding agents may also be used in histological applications. Alternatively, polynucleotide probes may be used within such applications.

As noted above, to improve sensitivity, multiple markers may be assayed within a given sample. It will be apparent that binding agents specific for different markers provided herein may be combined within a single assay. Further, multiple primers or probes may be used concurrently. The selection of markers may be based on routine experiments to determine combinations that results in optimal sensitivity. In addition, or alternatively, assays for tumor proteins provided herein may be combined with assays for other known tumor antigens.

In other aspects of the present invention, cell capture technologies may be used prior to detection to improve the sensitivity of the various detection methodologies disclosed herein. Example cell enrichment methodologies employ immunomagnetic beads that are coated with specific monoclonal antibodies to surface cell markers, or tetrameric antibody complexes, may be used to first enrich or positively select cancer cells in a sample. Various commercially available kits may be used, including DYNABEADS EPITHELIAL ENRICH (Dynal Biotech), STEMSEP (StemCell Technologies, Inc., Vancouver, BC), and ROSETTESEP (StemCell Technologies). The skilled artisan will recognize that other readily available methodologies and kits may also be suitably employed to enrich or positively select desired cell populations.

DYNABEADS EPITHELIAL ENRICH contains magnetic beads coated with monoclonal antibodies specific for two glycoprotein membrane antigens expressed on normal and neoplastic epithelial tissues. The coated beads may be added to a sample and the sample then applied to a magnet, thereby capturing the cells bound to the beads. The unwanted cells are washed away and the magnetically isolated cells eluted from the beads and used in further analyses. ROSETTESEP can be used to enrich cells directly from a blood sample and consists of a cocktail of tetrameric antibodies that target a variety of unwanted cells and crosslinks them to glycophorin A on red blood cells (RBC) present in the sample, forming rosettes. When centrifuged over Ficoll, targeted cells pellet along with the free RBC.

Once a sample is enriched or positively selected, cells may be further analyzed. For example, the cells may be lysed and RNA isolated. RNA may then be subjected to RT-PCR analysis using breast tumor-specific primers in a Real-time PCR assay as described herein.

In some embodiments, cell capture technologies may be used in conjunction with real-time PCR to provide a more sensitive tool for measuring the levels of expression of markers in cancer cells. Detection of breast cancer cells in bone marrow samples, peripheral blood, biopsies, and other samples is desirable for diagnosis and prognosis in breast cancer patients.

Some embodiments include making and using antibodies and fragments thereof specific to MRJ(L) protein. Methods of making polyclonal and monoclonal antibodies are well known. For example, monoclonal antibodies to epitopes of MRJ(L) can be prepared from murine hybridomas according to the classical method of Kohler, G. and Milstein, C., Nature 256:495 (1975) or any of the well-known derivative methods thereof.

In addition, antibody fragment preparations prepared from the produced antibodies are contemplated. “Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs (Complementarity Determining Region) of each variable domain interact to define an antigen-binding site on the surface of the V_{H-VL} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region.

### Pharmaceutical Compositions

Some embodiments relate to compositions capable of treating or ameliorating a cancer, such as breast, skin, or ovarian cancer, or other disorders such as Huntington's disease, and cardiomyocyte hypertrophic growth. In some embodiments, treating or ameliorating cancer can include increasing the levels of markers such as MRJ(L) and DKK-1 in the cell of a subject. In some embodiments, a composition can include a nucleic acid encoding at least a portion of the MRJ(L) polypeptide. At least a portion as used herein can refer to at least about 5%, 10%, 20%, 50%, 70%, 80%, 90%, 95%, 99%, and 100%. In some embodiments, a composition can comprise a polypeptide comprising the sequence of MRJ(L) or fragment thereof. A “therapeutically effective amount” is a quantity of a chemical composition (such as a nucleic acid construct, vector, or polypeptide) used to achieve a desired effect in a subject being treated.

In some embodiments, a pharmaceutical composition can include a nucleic acid encoding at least a portion of MRJ(L) operably linked to a regulatory sequence. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990), the disclosure of which is incorporated herein by reference in its entirety. Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc.

In some circumstances, it may be desirable to regulate expression of a transgene in a gene therapy vector. For example, different viral promoters with varying strengths of activity may be utilized depending on the level of expression desired. In mammalian cells, the CMV immediate early promoter is often used to provide strong transcriptional activation. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that may be used depending on the desired effect include SV40, RSV LTR, HIV-1 and HfV-2 LTR, adenovirus promoters such as from the EIA, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, promoters as follows may be used to target gene expression in other tissues.

Tissue specific promoters include in (a) pancreas: insulin, elastin, amylase, pdr-I, pdx-I, glucokinase; (b) liver: albumin PEPCK, HBV enhancer, alpha fetoprotein, apolipoprotein C, alpha-I antitrypsin, vitellogenin, NF-AB, Transthyretin; (c) skeletal muscle: myosin H chain, muscle creatine kinase, dystrophin, calpain p94, skeletal alpha-actin, fast troponin 1; (d) skin: keratin K6, keratin KI; (e) lung: CFTR, human cytokeratin IS (K 18), pulmonary surfactant proteins A, B and C, CC-10, Pi; (f) smooth muscle: sm22 alpha, SM-alpha-actin; (g) endothelium: endothelin-I, E-selectin, von Willebrand factor, TIE (Korhonen et al., 1995), KDR/flk-I; (h) melanocytes: tyrosinase; (i) adipose tissue: lipoprotein lipase (Zechner et al., 1988), adipsin (Spiegelman et al., 1989), acetyl-CoA carboxylase (Pape and Kim, 1989), glycerophosphate dehydrogenase (Dani et al., 1989), adipocyte P2 (Hunt et al., 1986); (j) blood: P-globin; and (k) mammary: MMTV, and whey acidic protein (WAP).

In certain embodiments, it may be desirable to activate transcription at specific times after administration of the gene therapy vector. This may be done with such promoters as those that may be regulated by hormone or cytokine. For example in gene therapy applications where the indication is in a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones are expected to be useful with the nucleic acids described herein. Cytokine and inflammatory protein responsive promoters that could be used include K and T Kininogen (Kageyama et al., 1987), c-fos, TNF-alpha, C-reactive protein (Arcone et al., 1988), haptoglobin (Oliviero et al., 1987), serum amyloid A2, C/EBP alpha, IL-1, IL-6 (Poli and Cortese, 1989), Complement C3 (Wilson et al., 1990), IL-8, alpha-1 acid glycoprotein (Prowse and Baumann, 1988), alpha-1 antitrypsin, lipoprotein lipase (Zechner et al., 1988), angiotensinogen (Ron et al., 1991), fibrinogen, c-jun (inducible by phorbol esters, TNF alpha, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), alpha-2 macroglobulin and alpha-I antichymotrypsin.

In some embodiments, it is envisioned that cell cycle regulatable promoters may be useful. For example, in a bi-cistronic gene therapy vector, use of a strong CMV promoter to drive expression of a first gene such as p16 that arrests cells in the G1 phase could be followed by expression of a second gene such as p53 under the control of a promoter that is active in the G1 phase of the cell cycle, thus providing a “second hit” that would push the cell into apoptosis. Other promoters such as those of various cyclins, PCNA, galectin-3, E2FI, p53 and BRCAI could be used.

It is envisioned that any of the promoters described herein, alone or in combination with another, may be useful depending on the action desired.

In addition, the promoters described herein should not be considered to be exhaustive or limiting, those of skill in the art will know of other promoters that may be used in conjunction with the nucleic acids and methods disclosed herein.

In some embodiments, the nucleic acids for producing or administering any of the MRJ(L) polypeptides described herein may contain one or more enhancers. Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Nucleic acid constructs encoding any MRJ(L) polypeptide described herein can be introduced in vivo as naked DNA plasmids. DNA vectors can be introduced into the desired host cells by methods known in the art, including but not limited to transfection, electroporation (e.g., transcutaneous electroporation), microinjection, transduction, cell fusion, DEAE dextran, calcium phosphate precipitation, use of a gene gun, or use of a DNA vector transporter (See e.g., Wu et al. J. Biol. Chem., 267:963-967, 1992; Wu and Wu J. Biol. Chem., 263:14621-14624, 1988; and Williams et al. Proc. Natl. Acad. Sci. USA 88:2726-2730, 1991). A needleless delivery device, such as a BIOJECTOR® needleless injection device can be utilized to introduce the therapeutic nucleic acid constructs in vivo. Receptor-mediated DNA delivery approaches can also be used (Curiel et al. Hum. Gene Ther., 3:147-154, 1992; and Wu and Wu, J. Biol. Chem., 262:4429-4432, 1987). Methods for formulating and administering naked DNA to mammalian muscle tissue are disclosed in U.S. Pat. Nos. 5,580,859 and 5,589,466, both of which are herein incorporated by reference in their entireties. Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931), the disclosures of which are incorporated herein by reference in their entireties.

Alternatively, electroporation can be utilized conveniently to introduce nucleic acid constructs encoding any MRJ(L) polypeptide described herein into cells. Electroporation is well known by those of ordinary skill in the art (see, for example: Lohr et al. Cancer Res. 61:3281-3284, 2001; Nakano et al. Hum Gene Ther. 12:1289-1297, 2001; Kim et al. Gene Ther. 10:1216-1224, 2003; Dean et al. Gene Ther. 10:1608-1615, 2003; and Young et al. Gene Ther 10:1465-1470, 2003). For example, in electroporation, a high concentration of vector DNA is added to a suspension of host cell (such as isolated autologous peripheral blood or bone marrow cells) and the mixture shocked with an electrical field. Transcutaneous electroporation can be utilized in animals and humans to introduce heterologous nucleic acids into cells of solid tissues (such as muscle) in vivo. Typically, the nucleic acid constructs are introduced into tissues in vivo by introducing a solution containing the DNA into a target tissue, for example, using a needle or trochar in conjunction with electrodes for delivering one or more electrical pulses. For example, a series of electrical pulses can be utilized to optimize transfection, for example, between 3 and ten pulses of 100V and 50 msec. In some cases, multiple sessions or administrations are performed.

Another well known method that can be used to introduce nucleic acid constructs encoding any MRJ(L) polypeptide described herein into host cells is particle bombardment (also know as biolistic transformation). Biolistic transformation is commonly accomplished in one of several ways. One common method involves propelling inert or biologically active particles at cells. This technique is disclosed in, e.g., U.S. Pat. Nos. 4,945,050, 5,036,006; and 5,100,792, all to Sanford et al., the disclosures of which are hereby incorporated by reference in their entireties. Generally, this procedure involves propelling inert or biologically active particles at the cells under conditions effective to penetrate the outer surface of the cell and to be incorporated within the interior thereof. When inert particles are utilized, the plasmid can be introduced into the cell by coating the particles with the plasmid containing the exogenous DNA. Alternatively, the target cell can be surrounded by the plasmid so that the plasmid is carried into the cell by the wake of the particle.

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner et. al. Proc. Natl. Acad. Sci. USA 84:7413-7417, 1987; Mackey, et al. Proc. Natl. Acad. Sci. USA 85:8027-8031, 1988; Ulmer et al. Science 259:1745-1748, 1993, the disclosures of which are incorporated herein by reference in their entireties). The use of cationic lipids can promote encapsulation of negatively charged nucleic acids, and also promote fusion with negatively charged cell membranes (Felgner and Ringold Science 337:387-388, 1989, the disclosure of which is incorporated by reference herein in its entirety). Particularly useful lipid compounds and compositions for transfer of nucleic acids are described in WO95/18863 and WO96/17823, and in U.S. Pat. No. 5,459,127, the disclosures of which are incorporated herein by reference in their entireties.

In some embodiments, the nucleic acid constructs encoding any MRJ(L) polypeptide described herein are viral vectors. Methods for constructing and using viral vectors are known in the art (See e.g., Miller and Rosman, BioTech., 7:980-990, 1992). Preferably, the viral vectors are replication defective, that is, they are unable to replicate autonomously in the target cell. In general, the genome of the replication defective viral vectors that are used within the scope of the present disclosure lack at least one region that is necessary for the replication of the virus in the infected cell. These regions can either be eliminated (in whole or in part), or be rendered non-functional by any technique known to a person skilled in the art. These techniques include the total removal, substitution (by other sequences, in particular by the inserted nucleic acid), partial deletion or addition of one or more bases to an essential (for replication) region. Such techniques can be performed in vitro (for example, on the isolated DNA).

In some cases, the replication defective virus retains the sequences of its genome that are necessary for encapsulating the viral particles. DNA viral vectors commonly include attenuated or defective DNA viruses, including, but not limited to, herpes simplex virus (HSV), papillomavirus, Epstein Barr virus (EBV), adenovirus, adeno-associated virus (AAV), Moloney leukemia virus (MLV) and human immunodeficiency virus (HIV) and the like. Defective viruses, that entirely or almost entirely lack viral genes, are preferred, as defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al. Mol. Cell. Neurosci., 2:320-330, 1991, the disclosure of which is incorporated herein by reference in its entirety), defective herpes virus vector lacking a glycoprotein L gene (See for example, Patent Publication RD 371005 A, the disclosure of which is incorporated herein by reference in its entirety), or other defective herpes virus vectors (See e.g., WO 94/21807; and WO 92/05263, the disclosures of which are incorporated herein by reference in their entireties); an attenuated adenovirus vector, such as the vector described by Stratford-Perricaudet et al. (J. Clin. Invest., 90:626-630 1992; La Salle et al., Science 259:988-990, 1993, the disclosure of which is incorporated herein by reference in its entirety); and a defective adeno-associated virus vector (Samulski et al., J. Virol., 61:3096-3101, 1987; Samulski et al., J. Virol., 63:3822-3828, 1989; and Lebkowski et al., Mol. Cell. Biol., 8:3988-3996, 1988, the disclosures of which are incorporated herein by reference in their entireties).

In some embodiments, the vectors encoding any MRJ(L) polypeptide described herein may be adenovirus vectors. Adenoviruses are eukaryotic DNA viruses that can be modified to efficiently deliver a nucleic acid of the disclosure to a variety of cell types. Various serotypes of adenovirus exist. Of these serotypes, preference is given, within the scope of the present disclosure, to type 2, type 5 or type 26 human adenoviruses (Ad 2 or Ad 5), or adenoviruses of animal origin (See e.g., WO94/26914 and WO2006/020071, the disclosures of which are incorporated herein by reference in their entireties). Those adenoviruses of animal origin that can be used within the scope of the present disclosure include adenoviruses of canine, bovine, murine (e.g., Mav1, Beard et al. Virol., 75-81, 1990, the disclosure of which is incorporated herein by reference in its entirety), ovine, porcine, avian, and simian (e.g., SAV) origin. In some embodiments, the adenovirus of animal origin is a canine adenovirus, such as a CAV2 adenovirus (e.g. Manhattan or A26/61 strain (ATCC VR-800)).

The replication defective adenoviral vectors may include the ITRs, an encapsidation sequence and the polynucleotide sequence of interest. In some embodiments, at least the E1 region of the adenoviral vector is non-functional. The deletion in the E1 region preferably extends from nucleotides 455 to 3329 in the sequence of the Ad5 adenovirus (PvuII-BglII fragment) or 382 to 3446 (HinfII-Sau3A fragment). Other regions can also be modified, in particular the E3 region (e.g., WO95/02697, the disclosure of which is incorporated herein by reference in its entirety), the E2 region (e.g., WO94/28938, the disclosure of which is incorporated herein by reference in its entirety), the E4 region (e.g., WO94/28152, WO94/12649 and WO95/02697, the disclosures of which are incorporated herein by reference in their entireties), or in any of the late genes L1-L5.

In other embodiments, the adenoviral vector has a deletion in the E1 region (Ad 1.0). Examples of E1-deleted adenoviruses are disclosed in EP 185,573, the contents of which are incorporated herein by reference. In another embodiment, the adenoviral vector has a deletion in the E1 and E4 regions (Ad 3.0). Examples of E1/E4-deleted adenoviruses are disclosed in WO95/02697 and WO96/22378, the disclosures of which are incorporated herein by reference in their entireties.

The replication defective recombinant adenoviruses can be prepared by any technique known to the person skilled in the art (See e.g., Levrero et al. Gene 101:195, 1991; EP 185 573; and Graham EMBO J., 3:2917, 1984, the disclosures of which are incorporated herein by reference in their entireties). In particular, they can be prepared by homologous recombination between an adenovirus and a plasmid, which includes, inter alia, the DNA sequence of interest. The homologous recombination is accomplished following co-transfection of the adenovirus and plasmid into an appropriate cell line. The cell line that is employed should preferably (i) be transformable by the elements to be used, and (ii) contain the sequences that are able to complement the part of the genome of the replication defective adenovirus, preferably in integrated form in order to avoid the risks of recombination. Examples of cell lines that can be used are the human embryonic kidney cell line 293 (Graham et al. J. Gen. Virol. 36:59, 1977, the disclosure of which is incorporated herein by reference in its entirety), which contains the left-hand portion of the genome of an Ad5 adenovirus (12%) integrated into its genome, and cell lines that are able to complement the E1 and E4 functions, as described in applications WO94/26914 and WO95/02697, the disclosures of which are incorporated herein by reference in their entireties. Recombinant adenoviruses are recovered and purified using standard molecular biological techniques that are well known to one of ordinary skill in the art.

In some embodiments, pharmaceutical compositions described herein comprise at least a portion of the MRJ(L) protein. In some embodiments, the MRJ(L) protein can be administered to a subject. In some embodiments, a portion of the MRJ(L) protein can be administered to a subject, where the portion contains biological activity useful to treat or ameliorate cancer. Methods to map regions in the MRJ(L) polypeptide with specific activity are well known and include techniques such as deletion analysis and mutagenesis analysis.

Some embodiments include pharmaceutical compositions comprising suitable carriers. While any suitable carrier known to those of ordinary skill in the art may be employed in the pharmaceutical compositions described herein, the type of carrier will typically vary depending on the mode of administration. Compositions described herein may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

In another illustrative embodiment, biodegradable microspheres (e.g., polylactate polyglycolate) are employed as carriers for the compositions described herein. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems. such as described in WO/99 40934, and references cited therein, will also be useful for many applications. Another illustrative carrier/delivery system employs a carrier comprising particulate-protein complexes, such as those described in U.S. Pat. No. 5,928,647, which are capable of inducing a class I-restricted cytotoxic T lymphocyte responses in a host.

The pharmaceutical compositions described herein will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions described herein may be formulated as a lyophilizate.

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles. Alternatively, a pharmaceutical composition may be stored in a freeze-dried condition requiring only the addition of a sterile liquid carrier immediately prior to use.

The development of suitable dosing and treatment regimens for using the particular compositions described herein in a variety of treatment regimens, including e.g., oral, parenteral, intravenous, intranasal, and intramuscular administration and formulation, is well known in the art, some of which are briefly discussed below for general purposes of illustration.

In certain applications, the pharmaceutical compositions described herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet. The active compounds may even be incorporated with excipients and used in the form of ingestible tablets, buccal tables, troches, capsules, elixirs, suspensions, syrups, wafers, and the like (see, for example, Mathiowitz et al., Nature 1997 Mar. 27; 386(6623):410-4; Hwang et al., Crit. Rev Ther Drug Carrier Syst 1998; 15(3):243-84; U.S. Pat. No. 5,641,515; U.S. Pat. No. 5,580,579 and U.S. Pat. No. 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations will contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. No. 5,543,158; U.S. Pat. No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. No. 5,756,353 and U.S. Pat. No. 5,804,212. Likewise, the delivery of drugs using intranasal microparticle resins (Takenaga et al., J Controlled Release 1998 Mar. 2; 52(1-2):81-7) and lysophosphatidyl-glycerol compounds (U.S. Pat. No. 5,725,871) are also well-known in the pharmaceutical arts. Likewise, illustrative transmucosal drug delivery in the form of a polytetrafluoroetheylene support matrix is described in U.S. Pat. No. 5,780,045.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol 1998 July; 16(7):307-21; Takakura, Nippon Rinsho 1998 March; 56(3):691-5; Chandran et al., Indian J Exp Biol. 1997 August; 35(8):801-9; Margalit, Crit Rev Ther Drug Carrier Syst. 1995; 12(2-3):233-61; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally difficult to transfect by other procedures, including T cell suspensions, primary hepatocyte cultures and PC 12 cells (Renneisen et al., J Biol Chem. 1990 Sep. 25; 265(27):16337-42; Muller et al., DNA Cell Biol. 1990 April; 9(3):221-9). In addition, liposomes are free of the DNA length constraints that are typical of viral-based delivery systems. Liposomes have been used effectively to introduce genes, various drugs, radiotherapeutic agents, enzymes, viruses, transcription factors, allosteric effectors and the like, into a variety of cultured cell lines and animals. Furthermore, the use of liposomes does not appear to be associated with autoimmune responses or unacceptable toxicity after systemic delivery. In certain embodiments, liposomes are formed from phospholipids that are dispersed in an aqueous medium and spontaneously form multilamellar concentric bilayer vesicles (also termed multilamellar vesicles (MLVs).

Alternatively, in other embodiments, the invention provides for pharmaceutically-acceptable nanocapsule formulations of the compositions of the present invention. Nanocapsules can generally entrap compounds in a stable and reproducible way (see, for example, Quintanar-Guerrero et al., Drug Dev Ind Pharm. 1998 December; 24(12):1113-28). To avoid side effects due to intracellular polymeric overloading, such ultrafine particles (sized around 0.1 μm) may be designed using polymers able to be degraded in vivo. Such particles can be made as described, for example, by Couvreur et al., Crit Rev Ther Drug Carrier Syst. 1988; 5(1):1-20; zur Muhlen et al., Eur J Pharm Biopharm. 1998 March; 45(2):149-55; Zambaux et al. J Controlled Release. 1998 Jan. 2; 50(1-3):31-40; and U.S. Pat. No. 5,145,684.

In further aspects of the present invention, the pharmaceutical compositions described herein may be used for the treatment of cancer, particularly for the treatment of breast cancer. Within such methods, the pharmaceutical compositions described herein are administered to a patient, typically a warm-blooded animal, preferably a human. A patient may or may not be afflicted with cancer. Accordingly, the pharmaceutical compositions described herein may be used to prevent the development of a cancer or to treat a patient afflicted with a cancer. Pharmaceutical compositions may be administered either prior to or following surgical removal of primary tumors and/or treatment such as administration of radiotherapy or conventional chemotherapeutic drugs. As discussed herein, administration of the pharmaceutical compositions may be by any suitable method, including administration by intravenous, intraperitoneal, intramuscular, subcutaneous, intranasal, intradermal, anal, vaginal, topical and oral routes.

In some embodiments, MRJ(L), MRJ(S), and fragments thereof may induce reversal of EMT. Some embodiments include methods for reversing EMT. For example, contacting a cell with a polypeptide comprising MRJ(L), MRJ(S), and fragments thereof, or a nucleic acid encoding a polypeptide comprising MRJ(L), MRJ(S), and fragments thereof. Reversal of EMT may increase drug sensitivity, increase sensitivity to radiation, decrease cell proliferation, and reducing stem-ness of cancer cells. In some embodiments, MRJ(L), MRJ(S), and fragments thereof may be agents that induce such changes. In some embodiments, administering MRJ(L), MRJ(S), and fragments thereof to a subject in need thereof can increase the efficiency of other treatments, such as chemotherapeutic drugs, and/or radiation therapy.

### Kits

Some embodiments relate to kits for use with any of the diagnostic methods described herein. Such kits typically comprise two or more components necessary for performing a diagnostic assay. Components may be compounds, reagents, containers and/or equipment. For example, one container within a kit may contain an antibody or fragment thereof that specifically binds to MRJ(L). Such antibodies or fragments may be provided attached to a support material, as described herein. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a tumor protein in a biological sample. Such kits generally comprise at least one oligonucleotide probe or primer, as described above, that hybridizes to a polynucleotide encoding a tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a marker.

In some embodiments kits can be used to diagnose the presence of a cancer, the stage of progression of a cancer, or the metastatic potential of a cancer. In some embodiments, the cancer can comprise breast, skin, or ovarian cancer.

### EXAMPLES

### Example 1

### MRJ(L) is Expressed at a Significantly Lower Level in Breast Cancer Cell Lines

The expression of MRJ(L) in breast cancer cell lines was assessed by a real-time qRT-PCR. MDA-MB-231 and MDA-MB-435 cells were maintained as described in Samant R. S. et al., “Analysis of mechanisms underlying BRMS1 suppression of metastasis.” Clin Exp Metastasis 2000, 18:683-693. Transfected cells were grown in presence of 500 μg/ml G418 (Invitrogen, CA, USA). MCF10.DCIS.com, MCF10CAc1.a, MCF10CAc1.d were cultured in DMEM/F12 with 5% horse serum, cholera toxin (100 ng/ml), insulin (10 μg/ml), hydrocortisone (500 μg/ml) and EGF (25 μg/ml). MCF7 was cultured in DMEM/F12 containing 5% horse serum and insulin (10 μg/ml). All cells were maintained at 37° C. with 5% CO₂ in a humidified atmosphere.

MCF10DCIS.com (locally aggressive) and metastatic variants MCF10CA c1.a and c1.d are isogenic cell lines derived from in vivo passages of MCF10AT (tumorgenic) in nude mice (Santner S J, et al., “Malignant MCF10CA1 cell lines derived from premalignant human breast epithelial MCF10AT cells. Breast Cancer Res Treat 2001, 65:101-110; Miller F R, et al., “MCF10DCIS.com xenograft model of human comedo ductal carcinoma in situ.” J Natl Cancer Inst 2000, 92:1185-1186). MDA-MB-435 was derived from the pleural metastases of a 46-year old woman with breast carcinoma. It forms progressively growing tumors that form metastases in the lungs and regional lymph nodes following injection into the mammary fat pads of 5 week old athymic nude mice (Price J. E. et al., “Tumorigenicity and metastasis of human breast carcinoma cell lines in nude mice.” Cancer Res 1990, 50:717-721).

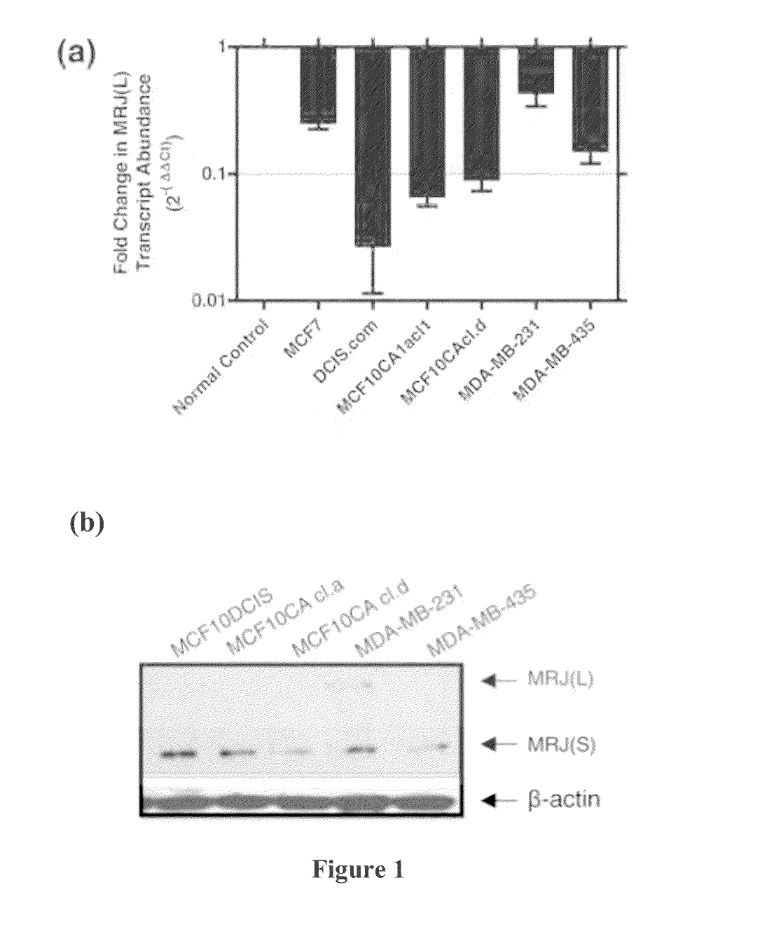

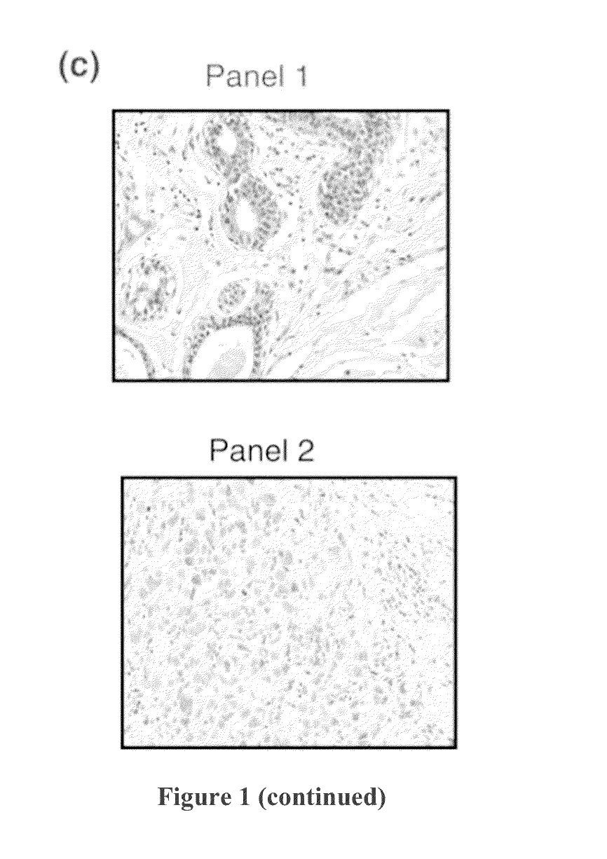

Referring to FIG. 1, panel (A), expression of mammalian relative of DnaJ (MRJ) long isoform (MRJ [L]) was significantly lower in various breast cancer cell lines as compared with that observed in RNA from normal breast. However among the cancer cell lines, MDA-MB-231 expressed MRJ(L) at a relatively high level. This observation was validated at the protein level by an immunoblot for MRJ. Real-time quantitative RT-PCR was used to assess expression of MRJ(L) relative to endorse control gene glyceraldehyde 3-phosphate dehydrogenase (GAPDH). The data are represented as fold change in the abundance of the MRJ(L) transcripts using commercially available normal breast RNA as a calibrator. Each reaction was carried out in triplicate, and the experiment was repeated once with RNA from the same cell lines at a different passage. The error bars represent the standard error.

Referring to FIG. 1, panel (B), an equal amount of protein lysate (20 ng) was resolved on SDS-PAGE and immunoblotted for levels of MRJ isoforms. Equal loading was confirmed by comparable β-actin signal. Corresponding to the qRT-PCR data, the larger isoform MRJ(L) (38.6 kd) was not detectable. Upon prolonged exposure, MDA-MB-231 cells showed weakly detectable MRJ(L).

### Example 2

### Expression of MRJ is Lost in Advanced Breast Cancer

A breast carcinoma progression tissue microarray was probed using antibody specific to MRJ. A breast carcinoma progression array (CC08-00-001) developed by Cybrdi, Inc. was stained by 1:100 dilution (10 μg/ml) of a DNAJB6 monoclonal antibody. Mouse IgG₁ was used for the isotype background control. Photomicrographs were taken in the area of most intense and diffuse staining for MRJ. The intensity of staining of tumor cells was assessed as 0 (no staining) to 3 (strongest possible intensity of staining). The immunoscore was derived as the product of the percentage of cells at each intensity and the corresponding intensity. The products were added to get an average immunoscore for the group.

The antibodies used were unable to distinguish between small and large isoforms. The results showed that 80% of the normal breast cases (4 out of 5) were positive for MRJ staining. In benign cases, nearly half of the tissues stained positive (48%), while in infiltrating ductal carcinoma (IDC) cases (grade I and grade II), 50% cases stained positive (6 out of 12 cases). Notably, only 1 out of 6 cases (17%) of IDC grade III stained positive whereas, all 6 cases of IDC with lymph node metastasis showed a complete absence of staining. FIG. 1, panel (C) shows intense nuclear and cytoplasmic staining in cystic hyperplasia (Panel C1). In contrast this staining is absent in infiltrating ductal carcinoma grade III (Panel C2). Table 2 shows immunostaining of MRJ is decreased in aggressive breast cancer specimens and outlines the average immunoscores which decrease with the aggressiveness of the tumor.

TABLE 2

| | Total | Stained | Percent | IHC |
|---|---|---|---|---|
| Tissue type | assessed | positive | positive | Scores |
| Normal | 5 | 4 | 80 | 1.6 |
| Benign | 25 | 12 | 48 | 0.54 |
| IDC I & II | 12 | 6 | 50 | 0.45 |
| IDC III | 6 | 1 | 17 | 0.108 |
| IDC with LN | 6 | 0 | 0 | 0 |
| Metastasis | | | | |

IDC = infiltrating ductal carcinoma (the roman numerals indicate stage)
LN = lymph node

MRJ expression is lost as IDC grades advance. Notably, total absence of MRJ staining in cases with lymph node metastasis was seen. The MRJ antibody used did not distinguish between isoforms, and so we cannot comment on the presence or absence of each of the isoforms. None of the IDCs had positive nuclear staining (n=24), whereas there was a higher number of positive nuclear staining in normal and benign (adenosis, simple hyperplasia, atypical hyperplasia) lesions (eight out of 17 stained). In fact, one of the ways in which a tumor can gain aggressive behavior is by spontaneous deletion or silencing of the MRJ gene, which will lead to absence of both isoforms. It is also likely that the expression of MRJ(S) is not ubiquitous in tissues, in contrast to the expression observed in cell lines and this isoform may play similar or independent role(s) in regulating tumor growth.

### Example 3

### MRJ(L) has a Functional Nuclear Localization Signal

PSORT II software predicted the presence of an NLS in MRJ(L) from amino acids 305-320 [KRKKQKQREESKKKK] (SEQ ID NO:28). An in-frame fusion of the NLS was constructed using requisite primers and PCR and also generated a mutant NLS by inserting point mutations. The mutated NLS sequence is RPDRPETTEESKKKK (SEQ ID NO:29). Both the wild type and mutated NLS were cloned into the pEGFP-N1 plasmid to generate an in-frame EGFP fusion, pEGFP-N1-MRJ(L)^{WT} and pEGFP-N1-MRJ(L)^{mut}, respectively. COST cells were transfected with pEGFP-N1-MRJ(L)^{WT} or pEGFP-N1-MRJ(L)^{mut}. Cells were visualized 32 hrs post transfection using a confocal microscope with 63α water immersion objective. MRJ(L)^{WT}-EGFP was predominantly in the nucleus while, the mutant NLS protein, MRJ(L)^{mut}-EGFP, shows a uniform distribution throughout the cell (FIG. 2, panel (B)). Similar results were observed when the MRJ(L)^{WT}-EGFP and the MRJ(L)^{mut}-EGFP were tested in breast cancer cell line MDA-MB-231.

Equal protein from nuclear (N) and cytoplasmic (C) extracts (20 μg) from MRJ(L) expressors of MDA-MB-435 and MDA-MB-231 were analyzed for presence of MRJ(L). HDAC1 was used as a marker to validate the purity of the nuclear fraction. β-tubulin was used as cytoplasmic marker. MRJ(L) migrates at the apparent molecular weight of 38 KDa, MRJ(S) at 28 KDa., HDAC1 at 55KDa. The ectopically expressed MRJ(L) also shows nuclear as well as cytoplasmic localization in MDA-MB-231 cells (FIG. 2, panel (C)). However MRJ(S) was found to be almost exclusively cytoplasmic.

Accordingly, the analysis of the predicted domains of MRJ(L) revealed that MRJ(L) has a nuclear localization signal. Confocal microscopy studies using the EGFP fusion of MRJ(L) with wild type or mutated NLS showed that a significant amount of MRJ(L) is localized to the nucleus whereas the mutation in NLS rendered it cytoplasmic. The continued presence of the MRJ(L)-NLS-mut in the nucleus can be explained by the possibility that there exists an additional cryptic NLS that the PSORT II software may not have identified. Alternatively, MRJ(L) lacking the NLS may still interact with another nuclear protein and could be transported into the nucleus while attached to the other protein.

### Example 4

### Analysis of In Vitro Attributes of Tumor Progression

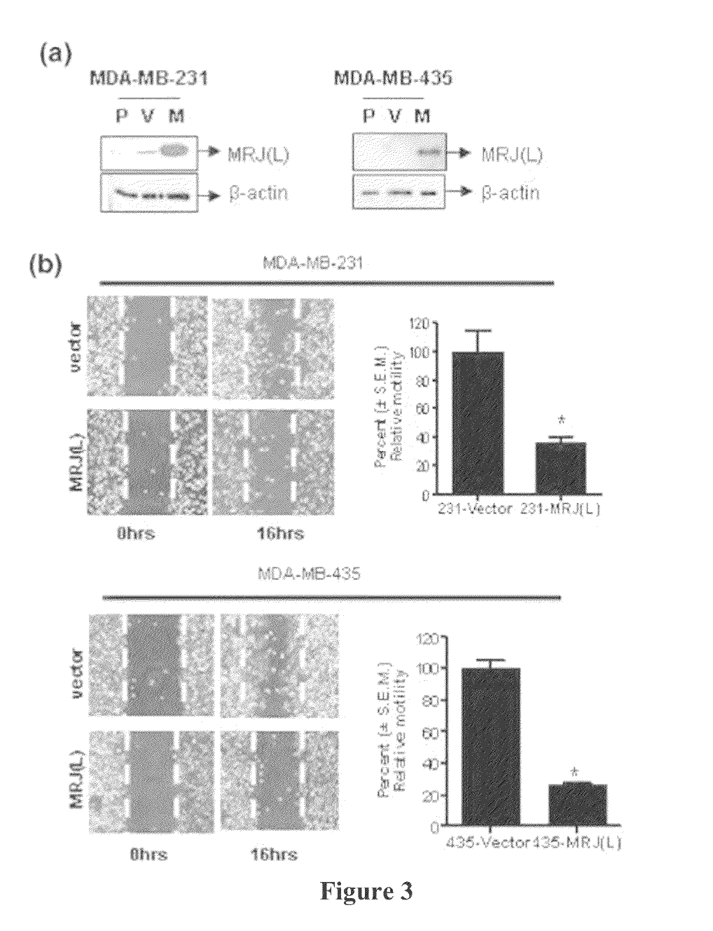

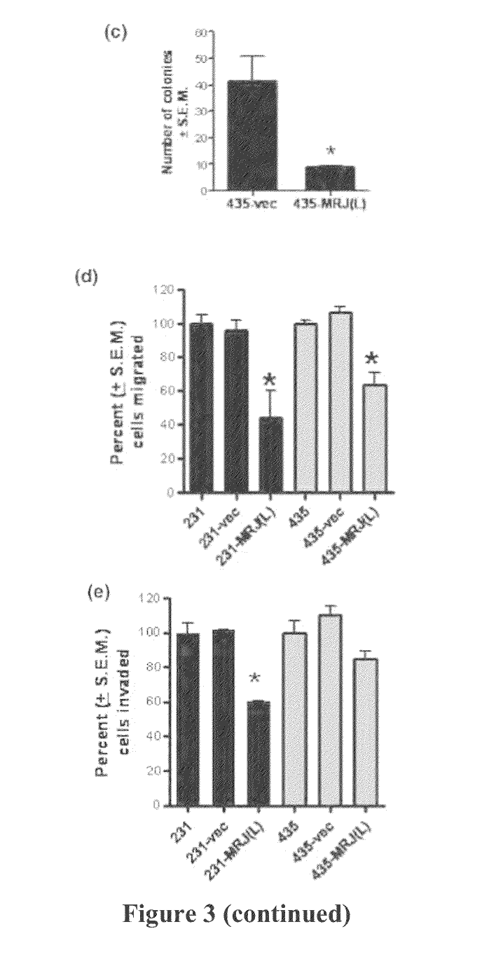

Cell lines ectopically-expressing stable MRJ(L) were established. MDA-MB-435 and MDA-MB-231 cells were stably transfected with MRJ(L)-pIRES2-EGFP as well as with the empty vector. The transfectants were FACS sorted to collect cells with highest fluorescence intensity (top 20%). Due to the presence of an IRES (internal ribosome entry site), the high fluorescence corresponds to high level of expression of MRJ(L). This FACS sorted population was expanded by propagation using G418 resistance and used for subsequent experiments. The expression of MRJ(L) was confirmed by western blot analysis where equal amounts (20 μg) of protein extracts from MRJ(L) expressors (M) of MDA-MB-231 and MDA-MB-435 were compared to the parent (P) and vector control (V) for the level of MRJ(L) (FIG. 3, panel (A)). MRJ(L) migrates at the apparent molecular weight of 38 KDa. Equal loading was confirmed by comparable β-actin signal.

The in vitro attributes of tumor progression were studied using wound healing assay, invasion, migration and soft agar colonization. Cells were cultured to confluence on pre marked 6-well plates. A central linear wound was made with a 200 μl sterile pipette tip. Phase micrographs of the wound cultures were taken at 0 and 16 h. The photographs were analyzed by measuring the distance from the wound edge of the cell sheet to the original wound site. The dotted white lines in the photomicrographs indicate the original position of the wound (FIG. 3, panel (B)). Migration activity was calculated as the mean of the distance between the edges in 12 independent fields per well. Each test group was assayed in triplicate, and the results are expressed relative to vector control cell migration, and (*) represents p<0.05. MDA-MB-231-MRJ(L) referred as 231-MRJ(L) and MDA-MB-435-MRJ(L) referred as 435-MRJ(L) showed decreased migration compared to the corresponding vector controls (231-vec and 435-vec) in wound healing assay. MRJ(L) expressors of MDA-MB-231 showed 50% motility in a wound healing assay compared to the vector control (FIG. 3, panel (B)). The trend was same for MRJ(L) expressors of MDA-MB-435 MRJ(L), which showed 30% motility (FIG. 3, panel (B)).

In soft agar assays, MDA-MB-435-MRJ(L) showed reduced anchorage independent growth compared to vector control. Cells (2×10³) suspended in 0.35% agar were plated onto a layer of 0.75% Bactoagar in DMEM-F12 (5% FBS) in 6-well tissue culture dishes. Visible colonies (>50 cells) were counted after 15 days with the aid of a dissecting microscope. The results displayed are number of colonies+/−S.E.M and (*) represents p<0.05 (FIG. 3, panel (C)). Expressors of MDA-MB-435 showed a highly reduced (10% of the vector control) ability for anchorage independent growth when tested by colony formation ability in soft agar. Moreover, the colonies that formed in MDA-MB-435-MRJ(L) were small and grew much slower than vector control (FIG. 3, panel (C)). The MDA-MB-231 cells did not show a significant change in the soft agar colonization although the overall trend appeared to be lower than vector control.

MRJ(L) expressors of MDA-MB-231 and MDA-MB-435 show significantly reduced ability to migrate through TransWell (FIG. 3, panel (D)) and are retarded in their ability to invade through Matrigel™-coated filters (FIG. 3, panel (E)). Migration and invasion assays were conducted using 8-nm polyethylene terpthalate filters. Cells migrated to the lower sides of the trans-well were stained using Diff-Quik® reagent and the cell number was counted under a microscope. Each test group was assayed in triplicate. Four different fields of each insert were photographed; each field was divided into quadrants and cells in diagonally opposite quadrants were counted. MRJ(L) expressors of MDA-MB-231 showed reduced capacity to migrate (40% of the vector control) and invade (60% of the vector control) through Matrigel™ (FIG. 3, panel (D), FIG. 3, panel (E)). The MRJ(L) expressors of MDA-MB-435 also showed reduced migration (60% of the vector control) but showed a modest decrease in invasion (85% of the vector control) (FIG. 3, panel (D), FIG. 3, panel (E)).

### Example 5

### MRJ(L) Retards Tumor Growth and Reduces Lung Colonization

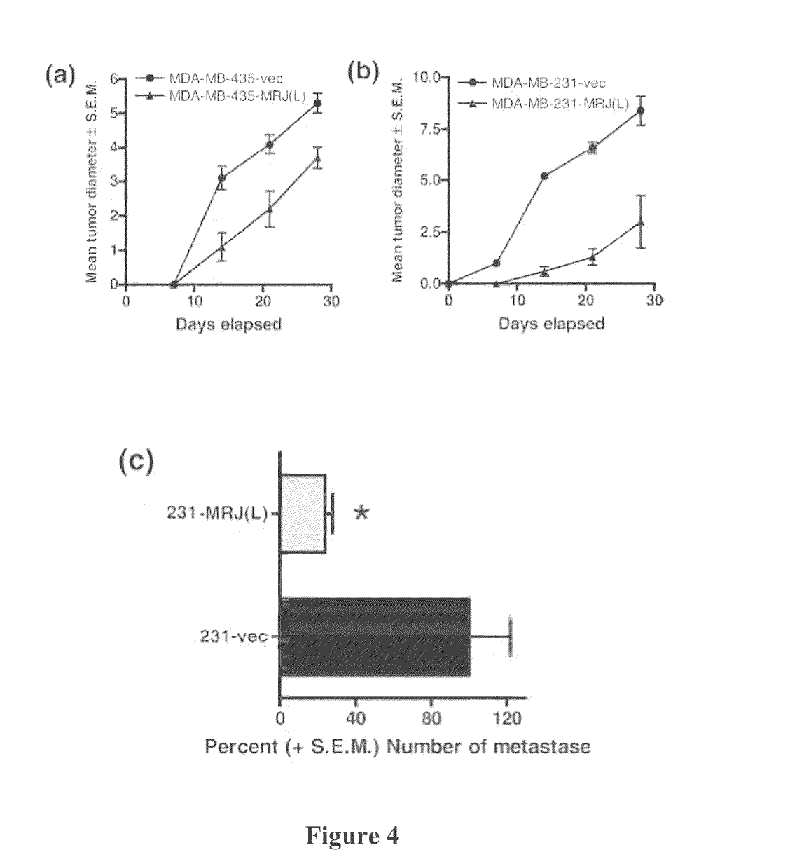

The MRJ(L) expressing MDA-MB-231 and MDA-MB-435 and the corresponding empty vector transfectants were independently assayed for orthotopic (mammary fat pad) tumor growth in nude mice. MRJ(L) expressors and corresponding vector control cells (10⁶/site) from MDA-MB-435 (FIG. 4, panel (A)) and MDA-MB-231 (FIG. 4, panel (B)) were injected in exposed axillary mammary fat pad of 6 week old, female athymic mice. Tumor size was measured weekly. 8 mice/group were used and the experiment was repeated once. The results are shown in FIG. 4, panel (A) and FIG. 4, panel (B) as the mean of the tumor diameters+/−SEM. The tumor growth of MRJ(L) expressors of both the cell lines were found to be retarded when compared to the empty vector control (FIG. 4, panel (A), FIG. 4, panel (B)).

An experimental metastasis assay (lung colonization assay) was performed by injecting 2.5×10⁵ MDA-MB-231 cells (in 0.2 ml) into the lateral tail vein of 3-4 week old, female athymic mice. After a period of 6 weeks lungs were removed and fixed with 20% Bouin's fixative in neutral buffered formalin before quantification of surface metastasis. 8 mice/group were used and the experiment was repeated once. The MRJ(L) expressing MDA-MB-231 cells showed a significantly decreased (p<0.05) ability to establish pulmonary metastases upon tail vein injection into athymic mice. The MRJ(L) expressing cells showed 65% fewer lung metastases compared to the vector control cells (FIG. 4, panel (C)).

### Example 6

### The Secreted Proteome of the MRJ(L) Expressors is Altered

Conditioned cell-free and serum-free medium from MRJ(L) expressing MDA-MB-435 was compared to the corresponding vector control. The analysis of secreted proteome was carried out using secreted proteome from MDA-MB-435. Proteins from the conditioned serum free medium were concentrated using a tC2 reversed-phase Sorbent column and digested with trypsin. The resulting peptides were analyzed by Electrospray tandem mass spectrometry (ESI-MS/MS) using a Q-TOF Ultima API-US mass spectrometer equipped with a nanoflow electrospray. The resulting data files were searched using an in-house MASCOT search engine. Ion scores higher than 35 (p<0.05) were considered as significant. Only proteins matching at least 2 peptides in Mascot were accepted (expectation: Zinc α-2-glycoprotein 1). MASCOT position indicates (inversely) a relative abundance of the protein in secretome (i.e. lower the score, higher is the abundance). Matched sequences correspond to the number of non redundant tryptic peptides mapped to the protein listed under the GenBank accession number. This indicates the confidence with which the protein was identified by the Mass Spectrometry. The alteration in the secreted proteome of MRJ(L) expressor of MDA-MB-435 is shown in Table 3.

TABLE 3

| | NCBI | MDA-MB-435 | | MDA-MB-435 vec | | MDA-MB-435-MRJ(L) | |
|---|---|---|---|---|---|---|---|
| Protein | Accession | MASCOT | Sequences | MASCOT | Sequences | MASCOT | Sequences |
| name | Number GI: | position | matched | position | matched | position | matched |
| VGF nerve | 17136078 | 1 | 16 | 2 | 21 | — | — |
| growth factor | | | | | | | |
| Secreted Phospho- | 4759166 | 5 | 5 | 7 | 7 | — | — |
| protein 1 (SPP1) | | | | | | | |
| Zinc α-2- | 4502337 | 43 | 1 | 32 | 1 | — | — |
| glycoprotein 1 | | | | | | | |
| Secreted protein, | 4507171 | 84 | 2 | 36 | 3 | — | — |
| acidic, cysteine | | | | | | | |
| rich (osteonectin) | | | | | | | |
| Nucleophosmin 1 | 10835063 | 22 | 3 | 46 | 1 | — | — |
| KiSS 1 metastasis | 29571104 | — | — | — | — | 14 | 2 |
| suppressor | | | | | | | |

MASCOT = a powerful search engine that uses mass spectrometry data to identify proteins from primary sequence databases
NCBI nr gi# = Gene identification number from a comprehensive, non-identical protein database maintained by National Center for Biotechnology Information

The changes in the secreted proteome of MRJ(L) expressor observed by mass spectrometry were confirmed by western blot analysis. Serum-free medium from equal number of MDA-MB-435 parent (P), vector (V) and MRJ(L) expressors (M) or MDA-MB-231 vector (V) and MRJ(L) expressors (M) was probed for presence of SPP1, SPARC, VGF, AZGP1. Equal amount of total protein extract (20 μg) of the same cells was probed for the level of KiSS1 simultaneously the expression of MRJ(L) was confirmed. β-actin was used to verify equal loading of the lysate. Apparent molecular weights of the proteins detected are represented in parenthesis: VGF (90KDa.), SPP1(62 KDa.), AZGP1 (47 K. Da.), SPARC (40 KDa.), NPM1 (37 K. Da), MRJ(L) (38 KDa.), KiSS1 (15 KDa)

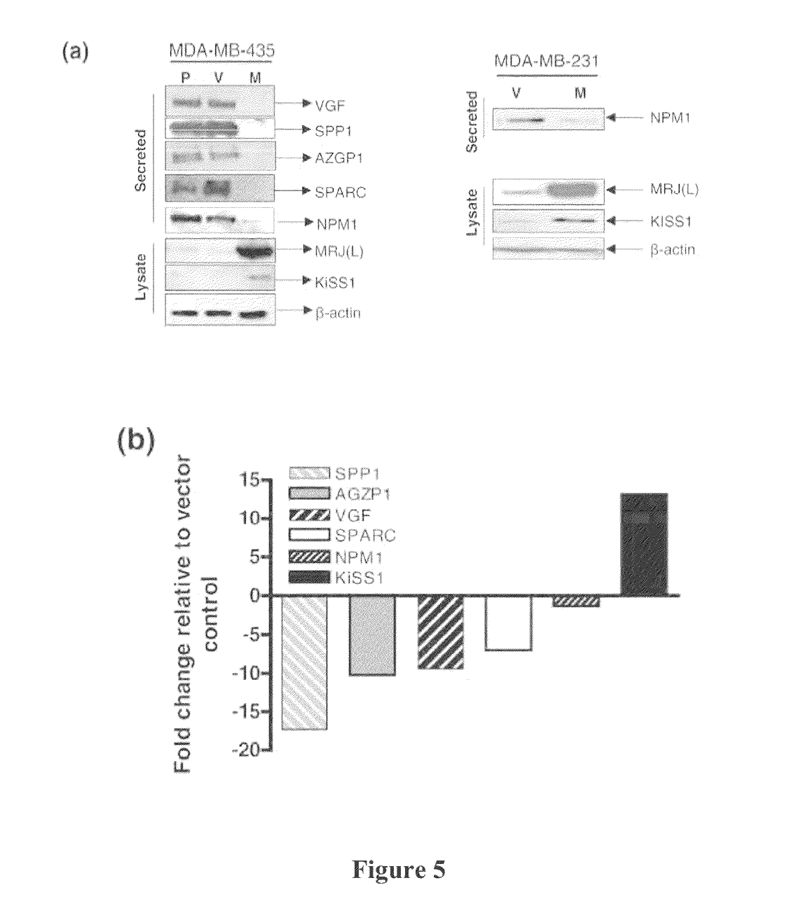

Osteopontin, osteonectin, zinc binding α-2-glycoprotein, VGF nerve growth factor and nucleophosmin were notably downregulated in MRJ(L) expressors. Conversely, the metastasis suppressor, KiSS1, was upregulated in the secreted proteome of MRJ(L)-expressing cells compared to the controls. Analysis of the serum-free medium by immunoblot analysis confirmed that osteopontin (SPP1), osteonectin (SPARC), VGF nerve growth factor (VGF), nucleophosmin (NPM1) and zinc binding α-2-glycoprotein 1 (AZGP1) levels were below detection limit in MDA-MB-435-MRJ(L) culture medium (FIG. 5, panel (A)). The increased expression of KiSS1 in MRJ(L) expressors was confirmed by analyzing the cell lysate (FIG. 5, panel (A)). MDA-MB-231 does not secrete OPN, SPARC, VGF or AZGP. However western blot analysis revealed increased level of KiSS1 expression and decreased level of NPM1 in MRJ(L) expressing MDA-MB-231.

### Example 7

### Determining the Changes in mRNA Level of the Key Secreted Proteins

The expression levels of SPP1, AZGP1, VGF, SPARC, NPM1 and KiSS1 was compared by quantitative RT-PCR to determine if the change in the levels of secreted proteome was due to change in transcription. Real-time qRT-PCR analysis was performed using RNA from the MDA-MB-435-vector and MDA-MB-435-MRJ(L) expressor. The PCR primers and probes for KiSS1, NPM1, AZGP1, SPARC, SPP1 and VGF and endorse control gene GAPDH were used. The reaction was performed for up to 40 cycles in triplicate. The gene expression Δ_{CT} values of mRNAs from each sample were calculated by normalizing with internal control GAPDH. The fold change is represented as 2^{-ΔΔcT}. Real-time qRT-PCR analysis was performed on the experimental mRNAs in triplicate and the experiment was repeated once from an independent passage. Real-time PCR comparison demonstrated a reduced expression of SPP1 (17 fold), AZGP (10 fold), VGF (9.4 fold), SPARC (7 fold), NPM1 (1.3 fold) and increased expression of KiSS1 (13 fold) in MDA-MB-435-MRJ(L) cells compared to the vector control (FIG. 5, panel (B)).

The quantitative-PCR analysis shows that the MRJ(L) influence on the secreted proteins is at the transcriptional level, suggesting that MRJ(L) plays a role in mediating regulation of transcription of these genes.

### Example 8

### Development of a MRJ(L)-Specific Antibody

To distinguish between MRJ(S) and MRJ(L), an antibody specific to MRJ(L) was developed. A KLH conjugated peptide containing amino acids 246-277 of MRJ(L) was used. Sequences corresponding to amino acids 246-277 of MRJ(L) are present in the J-domain of MRJ(L), but are absent from MRJ(S).

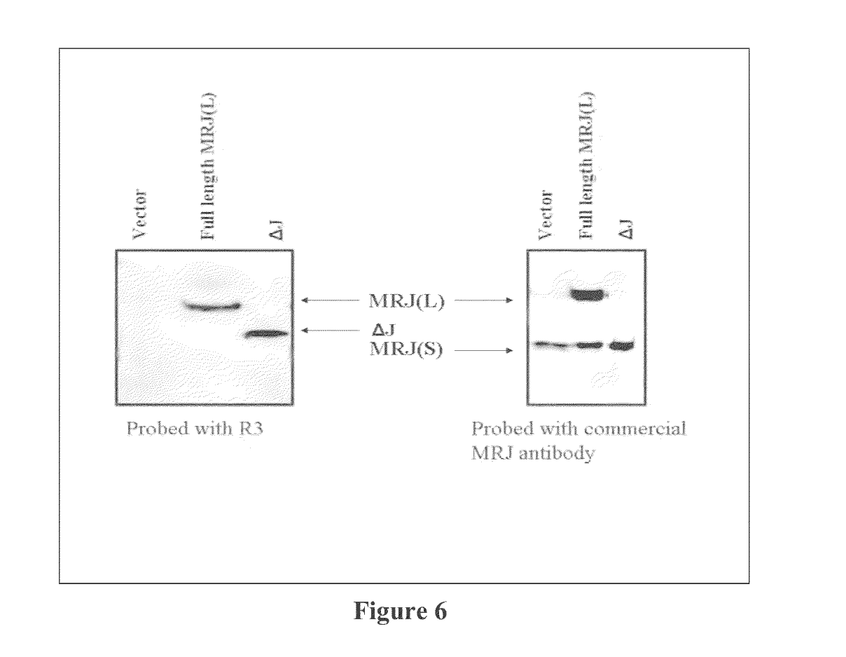

The peptide was injected into rabbits to raise a polyclonal antibodies specific to MRJ(L). In Western blot analysis, a test bleed from rabbit (R3), showed that it specifically recognized the MRJ(L) (FIG. 6). Western blots included protein lysates from MDA-MB-231 stably transfected with expression vectors containing: vector control; MRJ(L); or MRJ(L)ΔJ (where the J-domain of MRJ(L) has been deleted). Western blots were probed with the R3 test bleed, and the non-specific commercial MRJ antibody.

Test bleed analysis from rabbit R3 shows specific recognition of MRJ(L) and MRJ(L)ΔJ. However, the test bleed analysis from rabbit R3 shows and no cross reactivity to MRJ(S). The commercial MRJ antibody is designed against an epitope which is common to MRJ(L) and MRJ(S) and is located in the J-region of MRJ(L). FIG. 6 shows that the commercial MRJ antibody, recognizes MRJ(L) and MRJ(S), and MRJ(L)ΔJ.

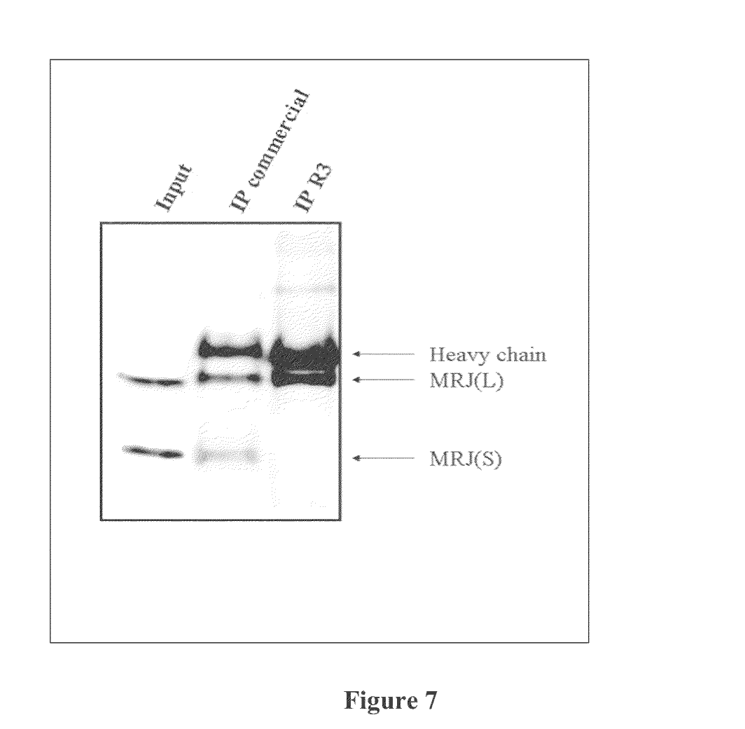

In addition, the ability of the test bleed from rabbit (R3) and the commercial MRJ antibody to immunoprecipitate MRJ(L) was compared. Total protein from MRJ(L) expressing MDA-MB-231 cells was immunoprecipitated with test bleed from rabbit (R3) and the commercial MRJ antibody. The immunoprecipitate was analyzed using western blot analysis (FIG. 7). Commercial MRJ antibody was used to probe this blot. As seen in FIG. 7, the lysate shows the large and small isoforms of MRJ. However, test bleed from rabbit (R3) specifically pulls down MRJ(L) and is incapable of precipitating MRJ(S).

The commercial antibody immunoprecipitated both the isoforms however the test bleed R3 immunoprecipitated only MRJ(L), confirming that we have successfully generated a MRJ(L) specific antibody. Accordingly, test bleed (R3) contains an antibody specific to MRJ(L) but not to MRJ(S).

### Example 9

### MRJ(L) Causes Reversal of Epithelial-Mesenchymal Transition (EMT)

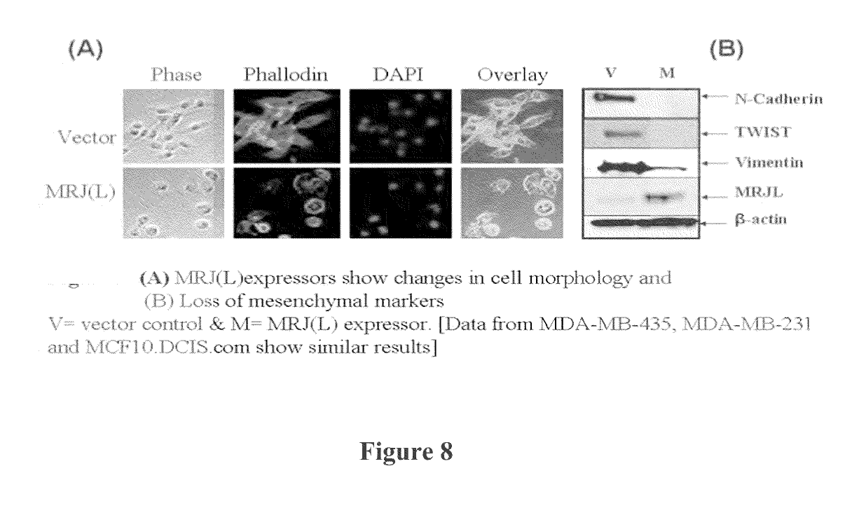

Stable expressors of MRJ(L) have a distinct appearance, they have lost the spindle shape and look rounded (FIG. 8, panel A and B). Hence we analyzed the MRJ(L) expressors for expression of mesenchymal markers viz. N-Cadherin, Vimentin, Twist. FIG. 8, panels A and B shows a substantial loss of expression of all these markers, suggesting a loss of mesenchymal phenotype concomitant with gain of an epithelial phenotype.

### Example 10

### MRJ(L) Upregulates DKK-1 Expression

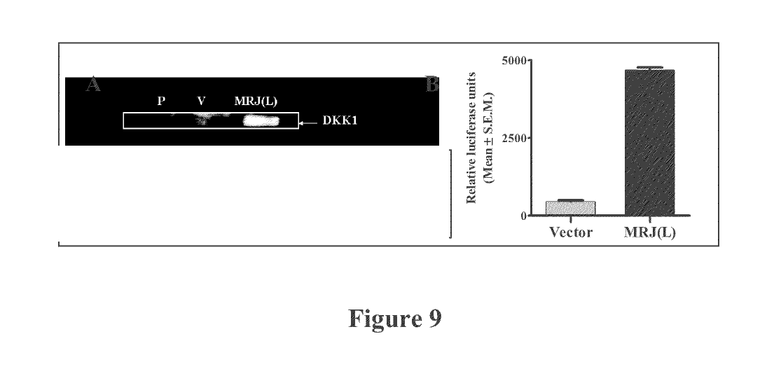

The Wnt pathway is known for its role in development as well as in EMT. mRNA expression profiling revealed that compared to the vector control, the MRJ(L) expressors showed a 10 fold upregulation in the expression of DKK-1, a known suppressor of Wnt pathway. Western blot analysis of MRJ(L) expressors for DKK-1 confirms the results (FIG. 9, panel (A)). A DKK-1-luciferase reporter activity was evaluated in stable MRJ(L) expressors and corresponding vector controls of MCF10DCIS.com, MCF10CA.c1.d and MDA-MB-435 cells. About 10 fold increase in the activity of DKK-1 promoter in MRJ(L)-expressing cells. FIG. 9, panel (B) shows results from breast cancer cell line MCF10CA.c1.d stably expressing MRJ(L).

### Example 11

### MRJ(L) Reduces the Level of β-Catenin

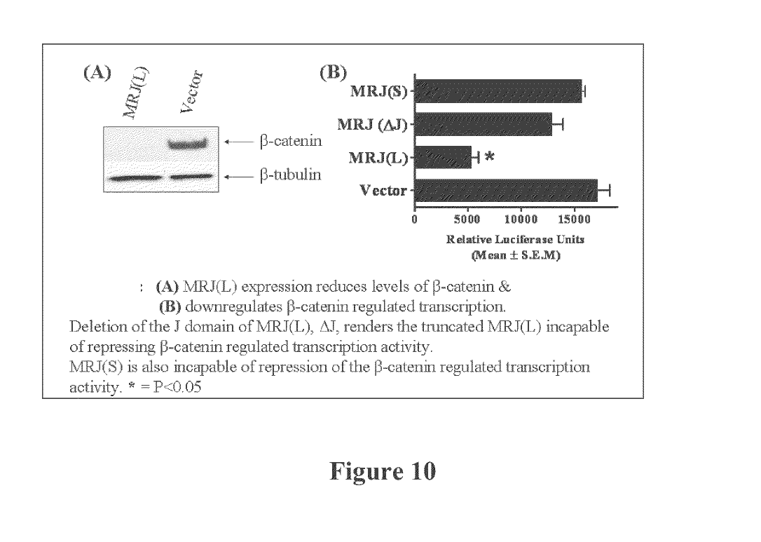

Upregulation of DKK-1 leads to inhibition of canonical Wnt signaling. This involves inactivation of Dishevelled; enabling the complex of axin, APC and active GSK-3β to phosphorylate β-catenin Phosphorylation of β-catenin targets it for degradation after ubiquitination. Thus, based on scientific rationale, we hypothesized that MRJ(L) expressing cells will show decreased levels of β-catenin. In agreement with the established pathway, the MRJ(L) expressors indeed showed reduced (almost not detectable) levels of β-catenin (FIG. 10, panel (A)).

Clone M50, Super8XTOPflash is a luciferase reporter of β-catenin-mediated transcriptional activation, generated and kindly provided by the laboratory of Dr. Randall T. Moon (Veeman M. T. et al, “Zebrafish prickle, a modulator of noncanonical Wnt/Fz signaling, regulates gastrulation movements” Curr Biol 13:680-5). For this construct, the backbone is the pTA-Luc vector (Clontech), which provides a minimal TA viral promoter driving expression of the firefly luciferase gene. Eight TCF/LEF binding sites were cloned into this vector. Using a transient luciferase assay, we found that MRJ(L) significantly repressed the reporter activity of Super8XTOPflash. Deletion of the J domain (MRJΔJ; Deleted for 75 amino acids corresponding to the J domain) renders MRJ(L) incapable of suppressing the activity of the luciferase reporter of β-catenin-mediated transcriptional activity (FIG. 10, panel (B)). This suggests that the domain of MRJ(L) involved in the suppression of the of β-catenin-mediated transcriptional activation lies within the deleted J domain.

Since MRJ(S) also has the identical J domain, we evaluated the capacity of MRJ(S) to repress Super8XTOPflash. FIG. 10, panel (B) shows that MRJ(S) was not capable of suppressing β-catenin-mediated transcriptional activity. This is likely due to the fact that MRJ(S), at ambient temperature, is in the cytoplasm and hence not capable of regulating transcription, an event that is nuclear. This suggests that MRJ(S) may not have a role in regulating the Wnt/β-catenin pathway.

### Example 12

### Nuclear Transport and Localization of MRJ

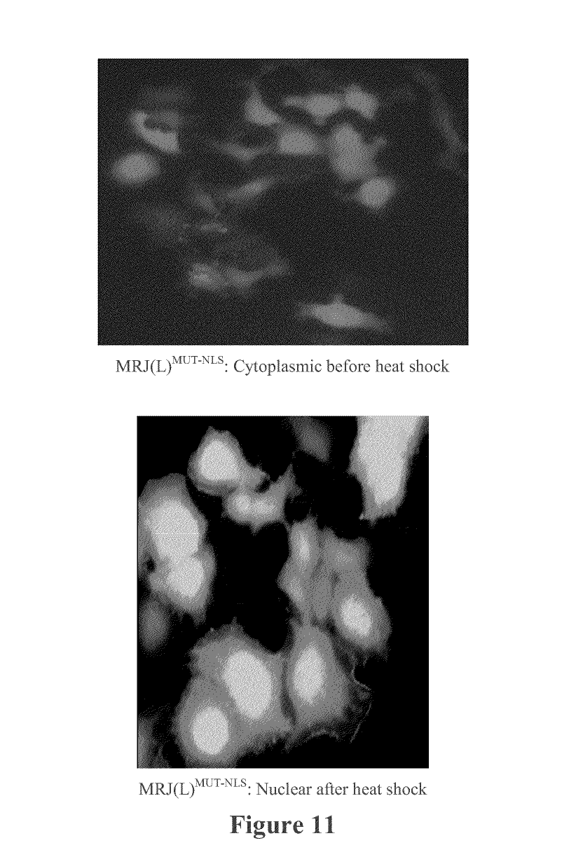

There are two isoforms of MRJ, namely, MRJ(L) which contains a nuclear localization signal (NLS) and is located in the nucleus, and MRJ(S) which has no NLS and is located in the cytoplasm. A pEGFP-N1 vector was used to generate reporter constructs containing MRJ(L) or MRJ(S). As described herein, pEGFP-N1-MRJ(L)^{WT} and pEGFP-N1-MRJ(L)^{mut} were transfected into COS7 cells. On analysis, MRJ(L)^{WT}-EGFP was predominantly in the nucleus while, the mutant NLS protein, MRJ(L)^{mut}-EGFP, shows a uniform distribution throughout the cell (FIG. 2, panel (B)). However, on heat shock at 42° C. of COS7 cells containing pEGFP-N1-MRJ(L)^{mut}, expressed protein localizes to the nucleus (FIG. 11).

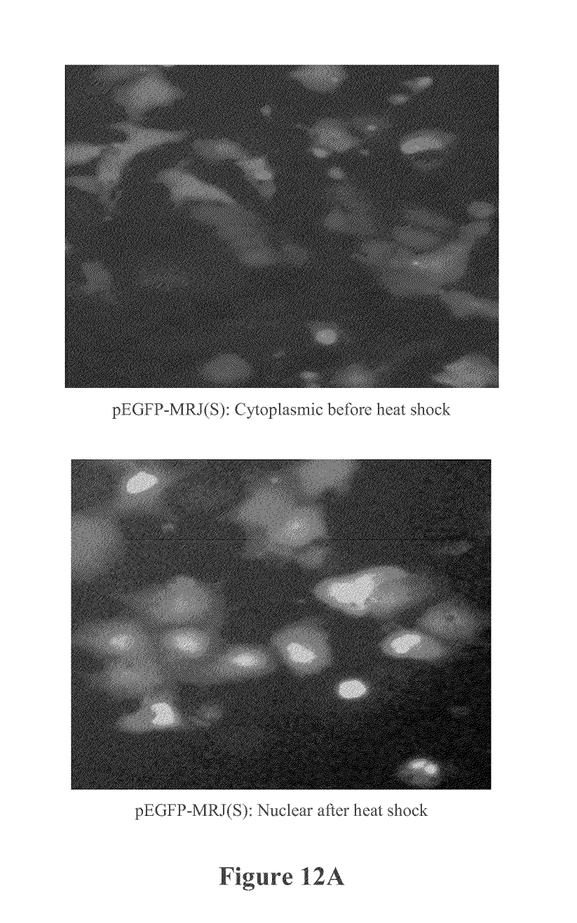

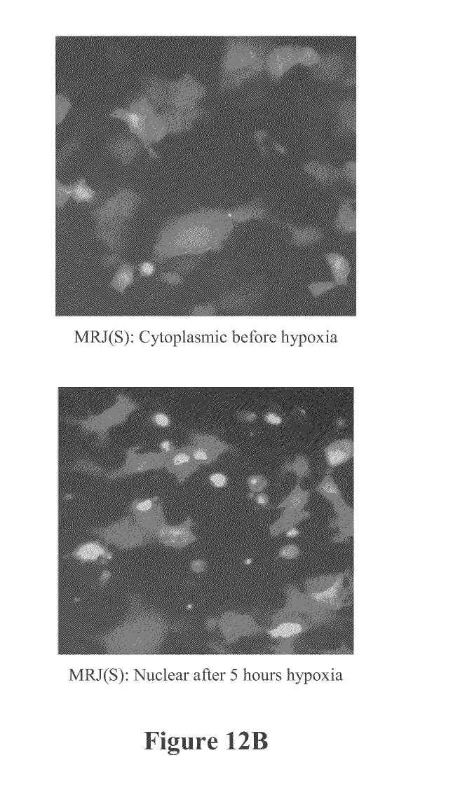

COS7 cells were transfected with pEGFP-N1-MRJ(S). Cells were treated with heat shock at 42° C., or hypoxia (2% O₂). Heat shock or hypoxia promoted nuclear localization of the expressed protein, MRJ(S)-EGFP from the cytoplasm (FIG. 12, panel (A) and FIG. 12, panel (B)). However, hypoxia treatment resulted in a lower amount of nuclear localization compared to heat shock treatment.

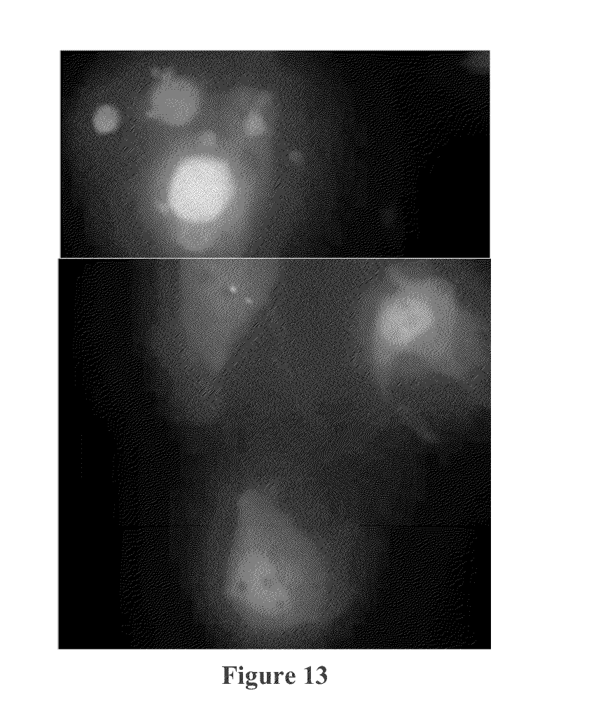

COS7 cells transfected with pEGFP-N1-MRJ(S) were treated with IFN-α (1000 μg/mL), IFN-γ (2000 μg/mL), IL-1α(10 μg/mL), or IL-2 (1000 μg/mL). Treatment with IFN-α, IFN-γ, IL-1α caused MRJ(S)-EGFP to localize to the nucleus. However, IL-2 did not cause any significant localization of MRJ(S)-EGFP to the nucleus. FIG. 13 shows transfected cells treated with IFN-γ. Table 4 summarizes the results. These results suggest that MRJ(S) has a stress-dependent non-classical nuclear localization mechanism.

TABLE 4

| MRJ Isoform | Experiment | Movement | Localization |
|---|---|---|---|
| MRJ(L)NLS- | Heat shock | Always in nucleus | High |
| WT | | | |
| MRJ(L)NLS- | Heat shock | Cytoplasm to nucleus | High |
| MUT | | | |
| MRJ(S) | Heat shock | Cytoplasm to nucleus | High |
| MRJ(S) | Hypoxia (5 hours) | Cytoplasm to nucleus | Medium |
| MRJ(S) | IFNα, IFNγ, | Cytoplasm to nucleus | Medium |
| | & IL-1α | | |
| MRJ(S) | IL-2 | No movement | None |

### Example 13

### Expression of MRJ in Melanoma

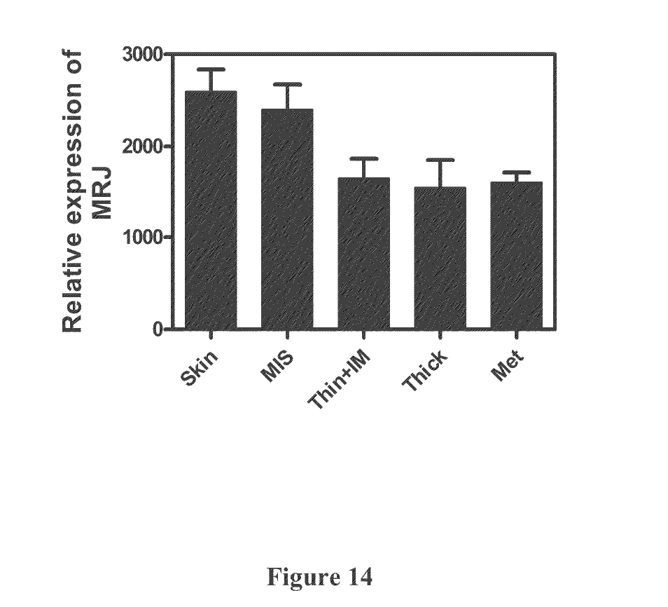

Gene microarray analysis (utilizing a Human Genome U133 Plus 2.0 array from Affymetrix, Inc.) was used to compare 40 metastatic melanoma samples. The metastatic melanoma samples included 22 bulky, macroscopic (replaced) lymph node metastases, 16 subcutaneous and 2 distant metastases (adrenal and brain), to 16 primary cutaneous melanoma specimens. The expression levels of MRJ decrease sharply with the progression of melanoma in situ (MIS) to primary cutaneous cancers of thin/intermediate thickness (Thin+IM) and are maintained at these low levels through progression of to the stage of metastatic melanoma (Thick, Met) (FIG. 14). Thin: thin melanomas (<1.5 mm in Breslow's thickness), IM: intermediate thickness (between 1.5-4.0 mm in Breslow's thickness), Thick: Melanomas (that are >4.0 mm in Breslow's thickness). These results shows that levels of MRJ (DNAJB6) decrease in the early stages of melanoma and are maintained at low levels as with progression to metastatic melanoma.

### Example 14

### MRJ(L) Expression and Changes in Cell Morphology

The morphology of cells were engineered to constitutively express MRJ(L) was compared to the corresponding vector control. MDA-MB-435 has been confirmed to be of melanoma origin (Rae, J. M., et al. (2007) Breast Cancer Res Treat 104, 13-19; Rae, J. M., et al. (2004) Clin Exp Metastasis 21, 543-552; Xie, X., et al. (1992) Clin Exp Metastasis 10, 201-210). However there still are convincing arguments about the lineage infidelity of this cell line (Chambers, A. F. (2009) Cancer Res 69, 5292-5293). MCF7 cells were cultured in DMEM/F12 containing 5% horse serum and insulin (10 μg/ml). MCF7 stably transfected with MRJ(L) were cultured in medium containing 500 μg/ml G418 (Life Technologies Inc.).

Cells cultured on cover slips were fixed in 4% paraformaldehyde in PBS for 20 min at room temperature and then permeabilized in PBS containing 0.1% Triton X-100 and 3% BSA. F-actin was stained with Phalloidin-TRITC (1:100, Molecular Probes, USA) for 1 h at room temperature and observed after mounting the cover slip in VECTASHIELD® (Vector Laboratories, Burlingame, Calif., USA). Cells were viewed using a Zeiss Axiovert 200 M microscope equipped with an Axiocam camera and Axiovision software (Carl Zeiss, Inc., Thornwood, N.Y.).

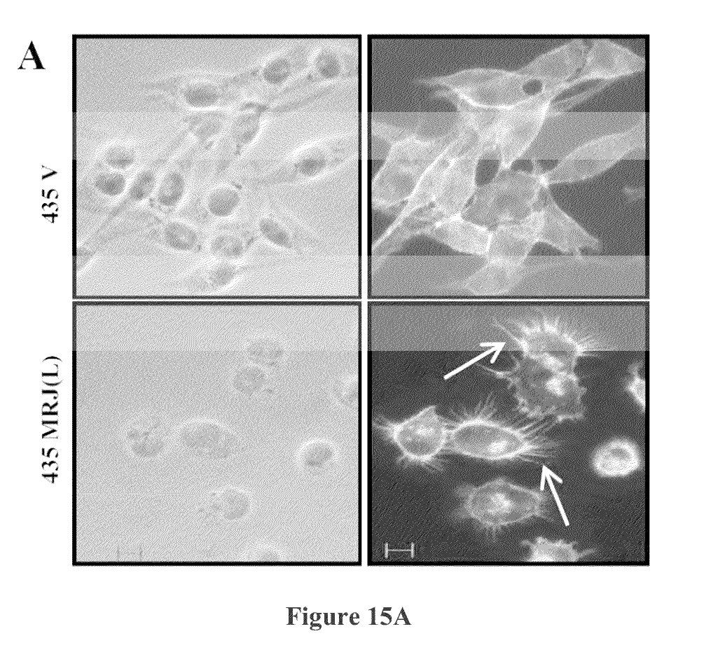

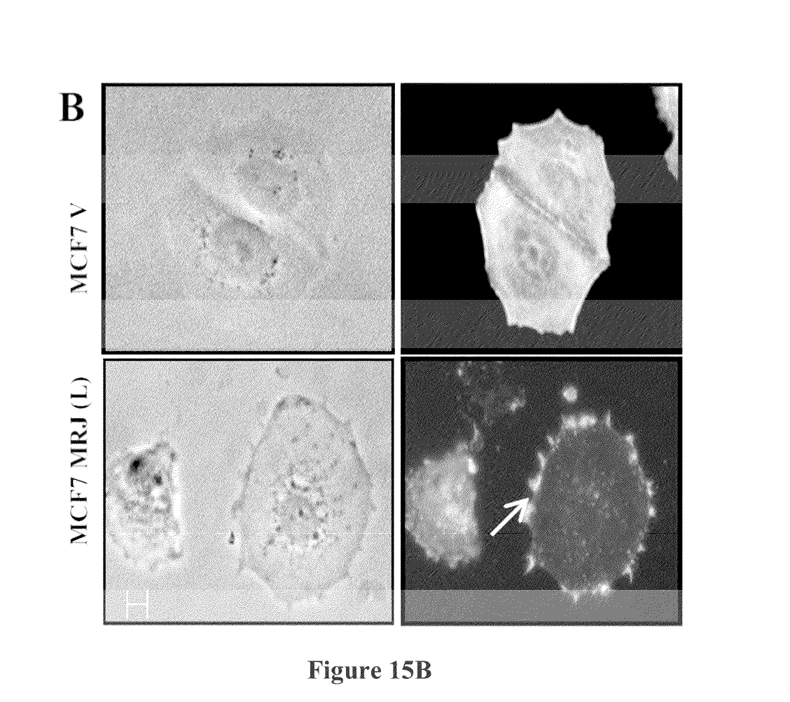

As shown in FIGS. 15A and 15B, MRJ(L) stable transfectants of MDA-MB-435 and MCF7 cells show a well spread/pavement like appearance and a differential actin staining when compared to the respective controls which look elongated and spindle shaped. The discrete morphological difference is highlighted by the extended filopodia very distinctly present on the surface of MRJ(L) expressors (FIGS. 15A and 15B, filopodia marked with arrows).

MRJ(L) expressors were observed to have a distinct epithelial-like appearance and increased expression of filopodia on the surface (FIGS. 15A and 15B). Recent studies have indicated that distinctive filopodia differing in length, positioning and dynamics are seen in different cells (Mattila, P. K., and Lappalainen, P. (2008) Nat Rev Mol Cell Biol 9, 446-454). Filopdia can act as sensors of the environment and change to lamellipodia when the cell decides to migrate (Azios, N. G., et al. (2007) Neoplasia 9, 147-158). Besides their role in cell migration, filopodia also have been reported to play a role in formation of adherens junctions between epithelial cells (Vasioukhin, V et al. (2000) Cell 100, 209-219). MRJ(L) expressing cells show numerous filopodia indicative of epithelial transformation which may be a prerequisite for the cells to search for an adjacent cell with extended filopodia leading to interdigitation of filopodia and formation of adherens junctions.

### Example 15

### MRJ(L) Expression Leads to Loss of Mesenchymal Markers and Gain in Epithelial Markers

MRJ(L) expression may influence the expression of characteristic EMT markers. Total protein extract was analyzed using western blot analysis from three different stable expressors of MRJ(L) cell lines: MDA-MB-435, MCF7 and MCF10CA1d.c1.1.

MCF10A is a spontaneously immortalized breast epithelial cell line. MCF10DCIS.com (locally aggressive) and metastatic variants MCF10CA1a.c1.1 and MCF10CA1d.c1.1 are isogenic cell lines derived from in vivo passages of MCF10AT. MCF10AT cells are transfected with T24 Ha-ras, and generate carcinomas in ˜25% of xenografts in nude mice (Strickland, L. B., et al. (2000) Breast Cancer Res Treat 64, 235-240; Santner, S. J., et al. (2001) Breast Cancer Res Treat 65, 101-110; Tait, L. R., et al. (2007) Int J Cancer 120, 2127-2134; and Miller, F. R., et al. (2000) J Natl Cancer Inst 92, 1185-1186).

Western Blot: Cell monolayers were washed twice with calcium and magnesium free PBS and lysed with cold lysis buffer (150 mM NaCl, 50 mM Tris, 1% NP-40 and protease and phosphatase inhibitors). The lysates were kept on ice for 1 h and centrifuged at 10,000 rpm for 30 mins at 4° C. Protein concentration was measured using Bradford reagent (Biorad Laboratories, Hercules, Calif., USA). Proteins were subjected to SDS-PAGE and transferred to PVDF membranes (0.2 μm). The membranes were blocked with 5% skimmed milk in TBST (1 M Tris pH 7.5, 9% NaCl, 0.5% Tween20) and incubated with primary antibodies overnight at 4° C. The membranes were then washed thrice with TBST and incubated with respective secondary antibodies for 1 h at room temp and then developed using SuperSignal™ (Pierce) following washes for 1 hr with three changes of TBST. For detection of MRJ (1:5000), 5% milk in PBS containing 0.2% Tween-20 was used as per manufacturer's instructions. DNAJB6 rabbit polyclonal (MOD and SNAI2 (Slug) antibodies were purchased from Abnova Corporation (Taipei City, Taiwan). Anti-Vimentin (1:500) was from Santa Cruz Biotechnologies Inc. (Santa Cruz, Calif., USA), HRP-β-actin (1:50,000, Sigma, USA). β-tubulin, KRT18, Twist1, β-catenin, antibodies were from Cell Signaling Technologies (Danvers, Mass., USA). Antibodies were used as per manufacturer's instructions. Total protein (20 μg) from MDA-MB-435-MRJ(L), MCF10CA1d.c1.1-MRJ(L) and MCF7-MRJ(L) and their corresponding vector controls was immunoblotted and analyzed for different levels of a variety of EMT markers.

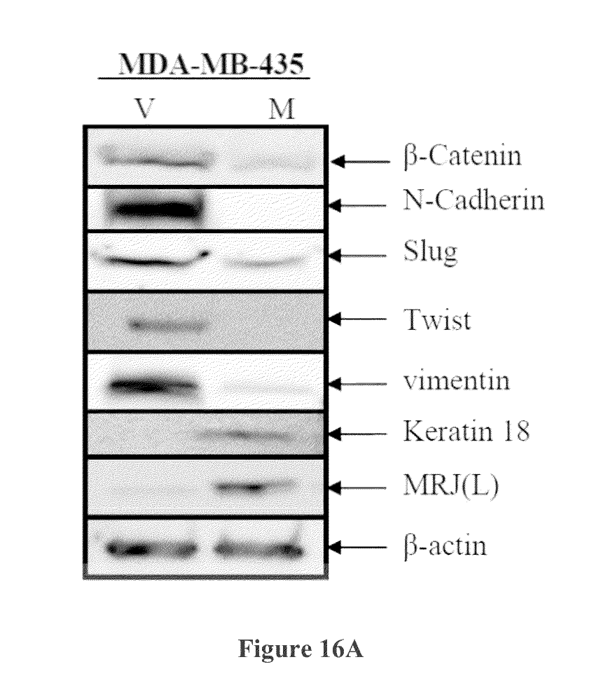

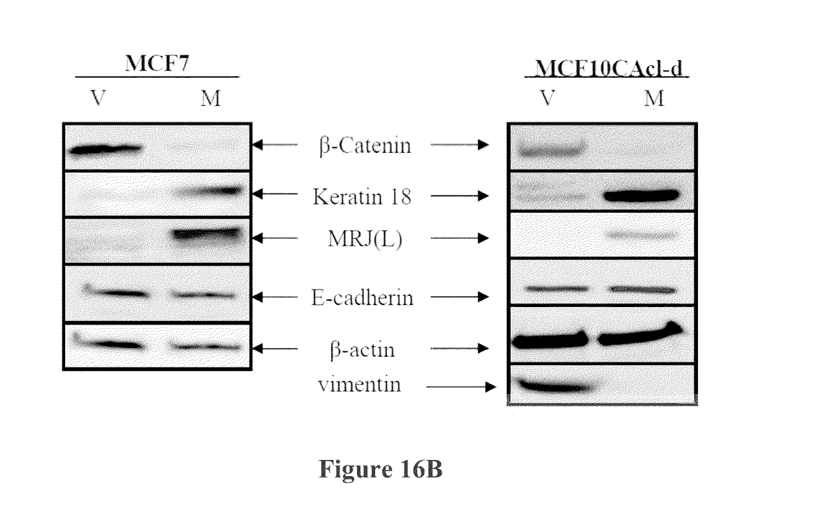

Referring to FIG. 16A, MRJ(L) expressors of MDA-MB-435 show loss of several mesenchymal markers viz. CTNNB1(β-catenin), VIM (Vimentin), CDH2 (N-cadherin), TWIST1, SNAI2 (SLUG) and gain of expression of epithelial marker KRT18 (Keratin 18). MCF7 cell line is negative for expression of Slug, Snail and Vimentin (Mironchik, Y., et al. (2005) Cancer Res 65, 10801-10809; and Sarrio, D., et al. (2008) Cancer Res 68, 989-997). MCF10CA1d.c1.1 is no differences in Slug or Twist however, showed a distinct loss of Vimentin expression upon MRJ(L) expression (FIG. 16B). There was no change detected in E-cadherin protein level in MCF7 and MCF10CA1d.c1.1 upon MRJ(L) expression (FIG. 16B). MDA-MB-435 is negative for E-cadherin. Loss of β-catenin and gain of Keratin18 was consistent in all the three cell lines.

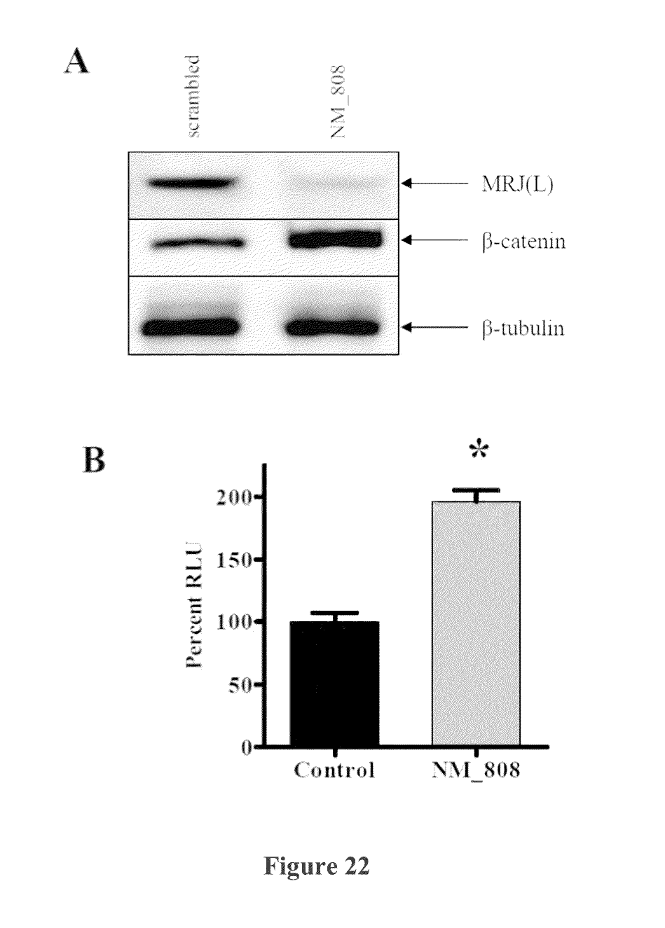

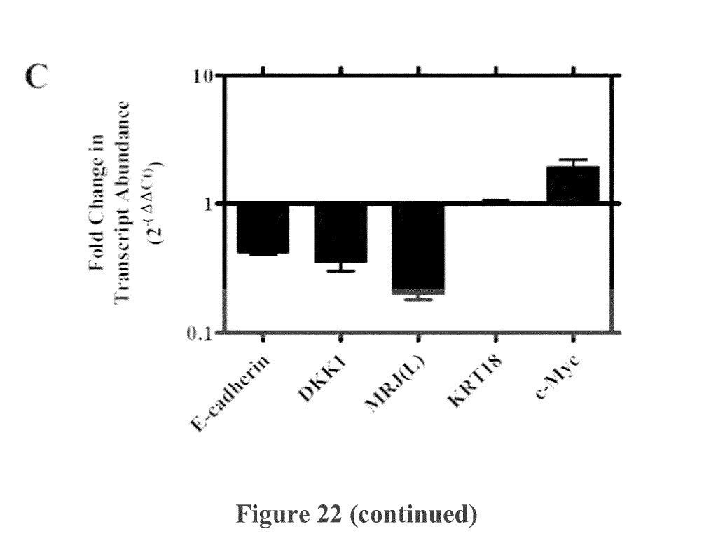

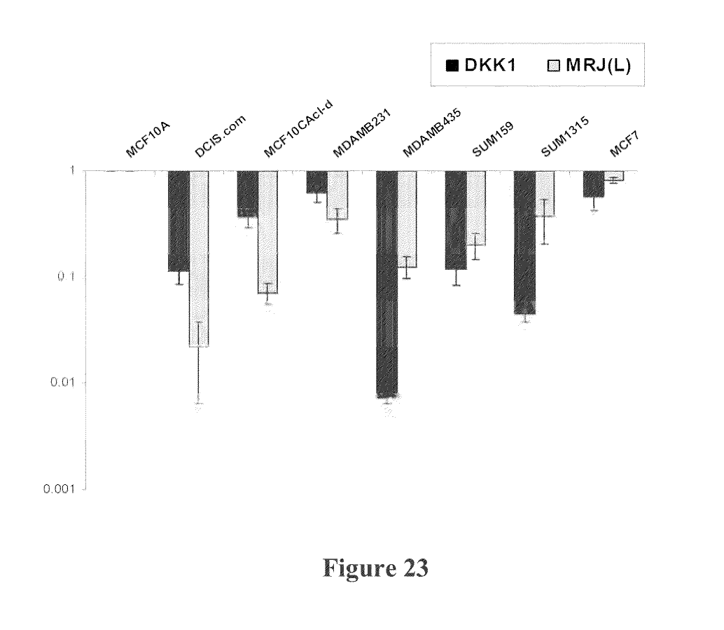

At the molecular level, protein levels of mesenchymal markers like CTNNB1 (β-catenin), VIM (Vimentin), CDH2 (N-cadherin), TWIST1, SLUG (SNAI2) were down regulated in MRJ(L) expressors, while the epithelial marker, KRT18 (Keratin 18) was upregulated (FIGS. 16A and 16B). No noticeable changes in E-cadherin levels (tested in MCF7 and MCF10CA1d.c1.1) were observed, but MCF10A cells did show a loss of E-cadherin transcript (CDH1) upon MRJ(L) knockdown (Example 21, FIG. 22, panels (A)-(C)). N-cadherin expression has been reported to increase motility of breast cancer cells regardless of their E-cadherin status (Nieman, M. T., et al. (1999) J Cell Biol 147, 631-644). Vimentin is a cytoskeletal protein which controls the formation of focal adhesions for efficient migration and invasion (Tsuruta, D., and Jones, J. C. (2003) J Cell Sci 116, 4977-4984; and Mclnroy, L., and Maatta, A. (2007) Biochem Biophys Res Commun 360, 109-114). Bühler et. al. have shown that forced expression of Keratin18 in aggressive breast cancer cells reduces Vimentin expression, alters cell shape and reduces invasion (Buhler, H., and Schaller, G. (2005) Mol Cancer Res 3, 365-371). Thus, downregulation of Vimentin, N-cadherin levels and upregulation of Keratin18 in MRJ(L) cells suggests a shift toward a less aggressive phenotype. Taken together, MRJ(L) is capable of up-regulating the expression of epithelial markers and down-regulating mesenchymal markers. Effectively, expression of MRJ(L) appears to be necessary, in part, to maintain the epithelial phenotype. Loss of MRJ(L) may be one of the triggers in tipping cells towards the metastable phenotype (Zavadil, J., Haley, J., Kalluri, R., Muthuswamy, S. K., and Thompson, E. (2008) Cancer Res 68, 9574-9577; and Klymkowsky, M. W., and Savagner, P. (2009) Am J Pathol 174, 1588-1593).

### Example 16

### Degradation of β-Catenin by MRJ(L) is Proteasome Dependent

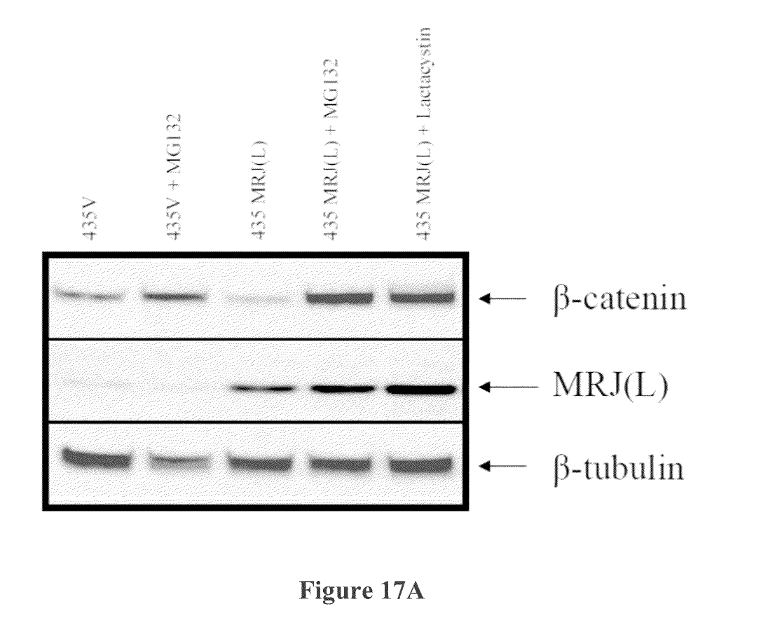

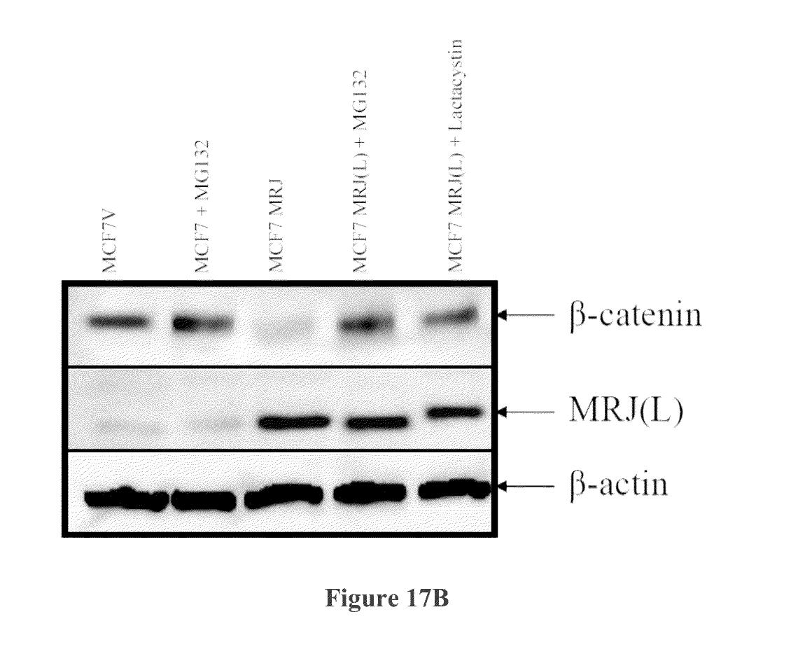

Stabilized β-catenin characteristically represents activated Wnt/β-catenin signaling. MRJ(L) expressors showed distinctly reduced levels of β-catenin protein. No significant change in the steady state mRNA levels for β-catenin was observed (data not shown). Whether this reduction was due to enhanced degradation of β-catenin was tested. MDA-MB-435MRJ(L) and MCF7-MRJ(L) and the corresponding vector controls, were treated with MG132 or lactacystin or vehicle (DMSO) control. Protein extract (40 μg) from the cells was analyzed for the level of β-catenin. β-tubulin was used to verify equal loading for MDA-MB-435 and β-actin was used to verify equal loading for MCF7. As shown in FIGS. 17A and 17B, the reduced levels of β-catenin in 435-MRJ(L) and MCF7-MRJ(L) were restored upon treatment with proteasome inhibitors MG132 or lactacystin. Thus, degradation of β-catenin by MRJ(L) is proteasome dependent.

### Example 17

### M RJ(L) Inhibits Wnt/β-Catenin Signaling

One of the prominent ways that active Wnt/β-catenin signaling manifests itself is by increased activation of TCF/LEF transcription factors. TCF/LEF luciferase reporter (M50-TOPFlash; (Veeman, M. T., et al. (2003) Curr Biol 13, 680-685)) activity in 435-MRJ(L), MCF7-MRJ(L) and MCF10CA1d.c1.1 was evaluated. Cells were transfected using Lipofectamine 2000 (Invitrogen) as per manufacturer's instructions. Total protein was harvested and luciferase activity measured using a Turner 20/20 luminometer (Turner Biosystems, Sunnyvale, Calif., USA). The luciferase reading was normalized to the total protein concentration. Data is expressed as relative luciferase activity, where control is 100%.

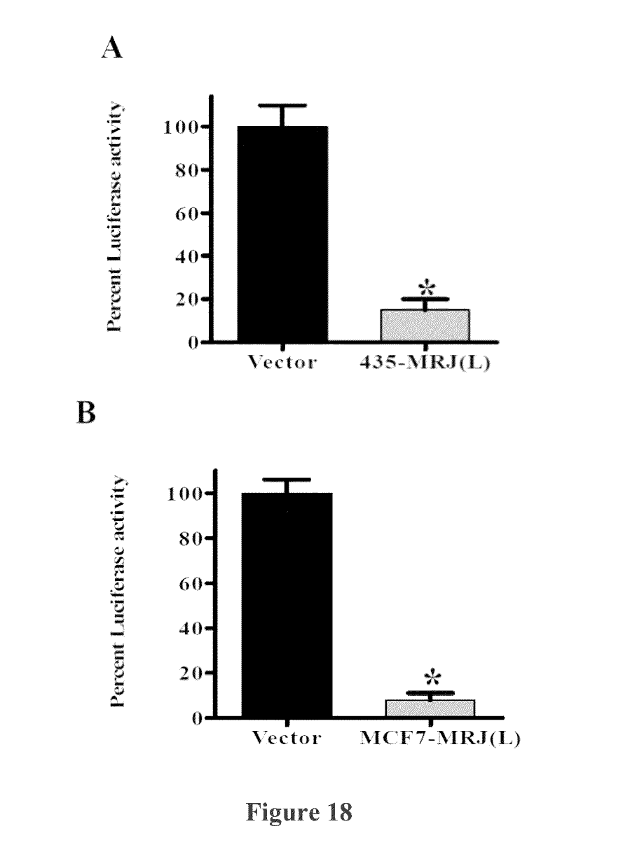

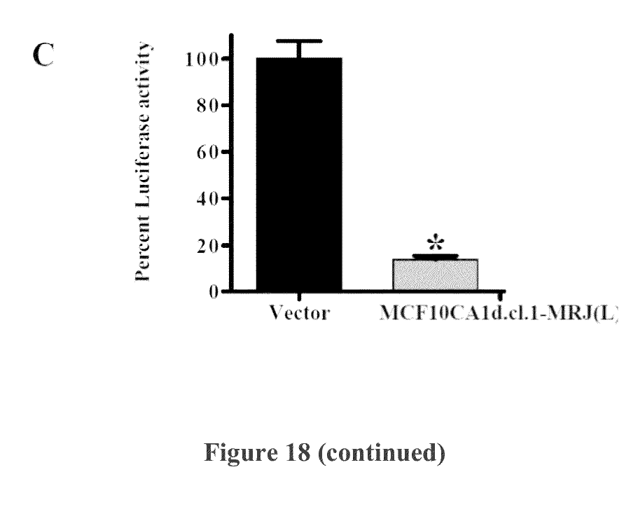

Referring to FIG. 18, panel (A), 18, panel (B), and 18, panel (C), a greater than 85% reduction in the TOPFlash activity in all the cell lines was observed, indicating a significant inhibition of the Wnt/β-catenin signaling.

### Example 18

### MRJ(L) Up Regulates DKK1

DKK1 is a secreted inhibitor of Wnt/β-catenin signaling which blocks Wnt signaling by binding to the Wnt co-receptor Lrp5/6. QRT-PCR analysis of different breast cancer cell lines showed that all of them express lower levels of DKK1 mRNA as compared to non-tumorigenic MCF10A cells.

RNA isolation and real-time quantitative RT-PCR: TRIzol reagent (Invitrogen) was used to isolate total RNA from cultured cells. RNA was treated with DNase I (Promega, Madison, Wis., USA). cDNA synthesis was carried out using a cDNA synthesis kit (Applied Biosystems Inc., Foster City, Calif., USA) with 1 μg total RNA as the template and random primers. Real-time quantitative RT-PCR (QRT-PCR) analysis was performed on the experimental mRNAs. The PCR primers and probes for Dickkopf1 (DKK1), DNAJB6 (MRJ(L)), c-Myc, E-cadherin, Keratin 18, and endorse control gene glyceraldehyde 3-phosphate dehydrogenase (GAPDH) were purchased from Applied Biosystems Inc. QRT-PCR was performed on an ABI 7500 HT instrument (Applied Biosystems). The gene expression ΔCt values of mRNAs from each sample were calculated by normalizing with endorse control, GAPDH and relative quantitation values were plotted using Graphpad Prism® (La Jolla, Calif.). QRT-PCR analysis was performed on the experimental mRNAs in triplicate, and the experiment was repeated once from an independent passage to confirm the findings.

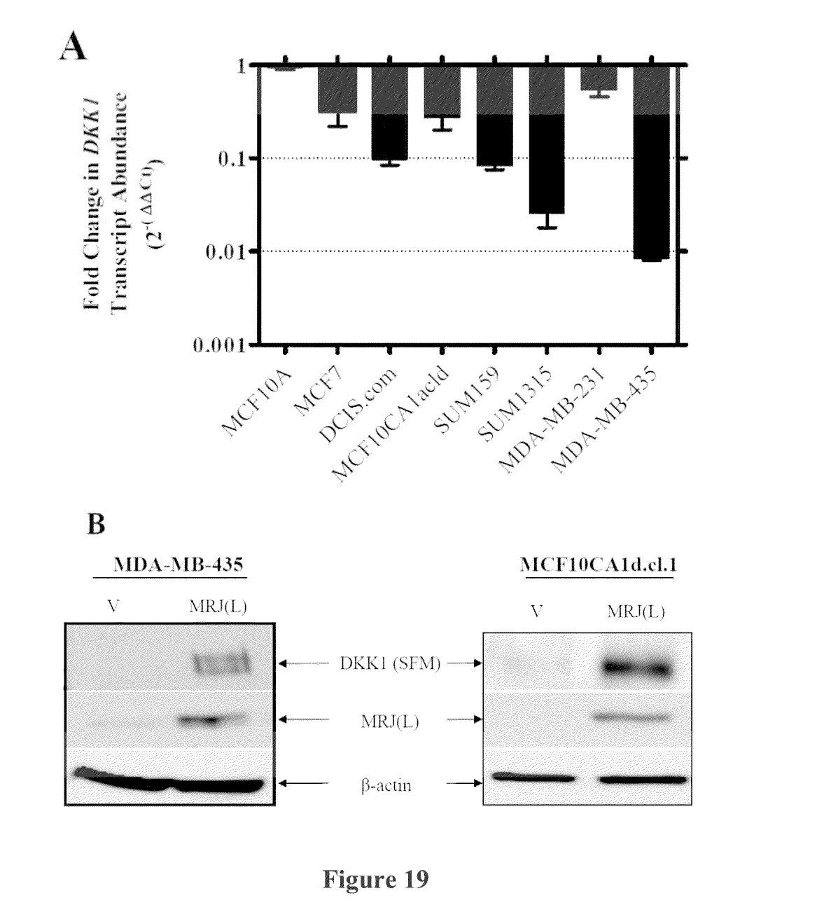

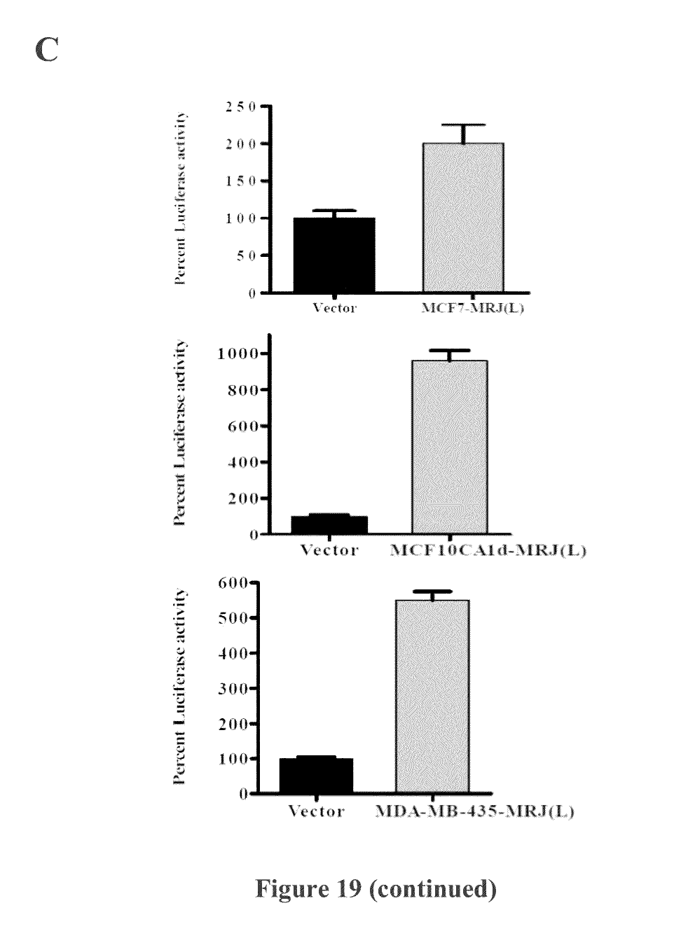

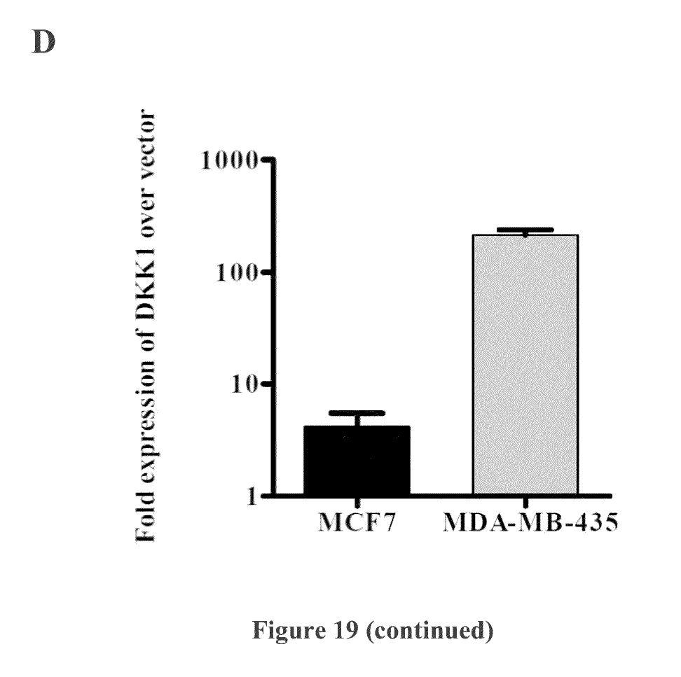

SUM 159 cells were cultured in Ham's F-12 with 5% FBS supplemented with insulin (5 mg/ml) and hydrocortisone (1 mg/ml). The cell line SUM 1315 was cultured in Ham's F-12 with 5% FBS supplemented with insulin (5 mg/ml) and EGF (10 μg/ml). Both SUM lines were bought from Asterand plc, Detroit, Mich., USA. SUM1315, SUM159, MCF10.DCIS.com express very low levels of DKK1 whereas, MCF7 and MDA-MB-231 cells seem to express slightly higher levels. The levels of DKK1 in MCF10CA1d.c1.1 cells were intermediate (FIG. 19, panel (A)). DKK1 mRNA levels in MDA-MB-435 were also evaluated and found it to be very low. The MRJ(L) expressors were also examined for expression of DKK1. As seen in FIG. 19, panel (B), a substantial increase was observed in the levels of secreted DKK1 in serum free conditioned medium from 435-MRJ(L) and MCF10CA1d.c1.1-MRJ(L) cells. These results corroborate with the increased DKK1 transcript observed by QRT-PCR (FIG. 19, panel (D)).

To determine if the increase in DKK1 is at transcriptional level, the activity of the luciferase reporter construct pDKK1-707 in MCF10CA1d.c1.1-MRJ(L), MCF7-MRJ(L), MDA-MB-435-MRJ(L) was assessed and compared to the corresponding vector control. pDKK-707 is a luciferase construct encompassing −707 to +43 base pairs of human DKK1 promoter (Chamorro, M. N., et al. (2005) Embo J 24, 73-84).

As seen in FIG. 19, panel (C), a 2.5-fold upregulation was observed of DKK1 promoter activity in MCF7-MRJ(L) and a 10-fold upregulation in MCF10CA1d.c1.1-MRJ(L) and 6 fold upregulation in MDA-MB-435.

### Example 19

### Silencing of DKK1 from MRJ(L) Expressors Leads to Restoration of β-Catenin

DKK1 binding to the Lrp5/6 receptors directs β-catenin to phosphorylation and subsequent proteosome mediated degradation. If the degradation of β-catenin in MRJ(L) expressing cells is due to the elevated DKK1 levels, silencing DKK1 expression in MRJ(L) expressing cells should lead to increased β-catenin levels. 435-MRJ(L) were treated with DKK1siRNA and compared their β-catenin levels with 435-MRJ(L) cells independently treated with control siRNA.

shRNA knockdown: Oligo Design Tool™ (Oligoengine, Seattle, Wash., USA) was used to design two shRNAs, NM_{—}807 and NM_{—}808, predicted to silence the expression of MRJ(L). The heteroduplexes supplied as asymmetric oligomers were annealed following the manufacturer's instructions and cloned into pSUPERIOR.neo+GFP (Oligoengine) at the BglII and HindIII sites. Recombinants were analyzed by restriction digestion using EcoRI and HindIII (Promega) and recombinant DNA was prepared using the QIA Maxiprep kit (Qiagen, Valencia, Calif., USA). MCF10A cells were transfected with shRNA targeting MRJ(L) (NM_{—}808) or a control non-targeting shRNA using Lipofectamine 2000 according to manufacturer's instructions. The extent of knockdown was assessed using QRT-PCR, using RNA isolated 32 hrs after transfection. siRNA knockdown: Cells were transiently transfected with SMARTpool® siRNA (Dharmacon, Chicago, Ill.) designed to target DKK1 or non-targeting siRNA control using Dharmafect1 (Dharmacon) following manufacturer's protocol. Conditioned serum free medium and lysates were collected from both, control and DKK1-siRNA treated cells 38 hrs post transfection. The extent of knockdown was assessed using western blot analysis. The serum free-cell free-supernatant (SFM) was harvested from equal number of cells, concentrated to 10% of its original volume and immunoblotted for DKK1. Total protein (35 μg) was immunoblotted and analyzed for the levels of 3-catenin β-tubulin was used to verify equal loading.

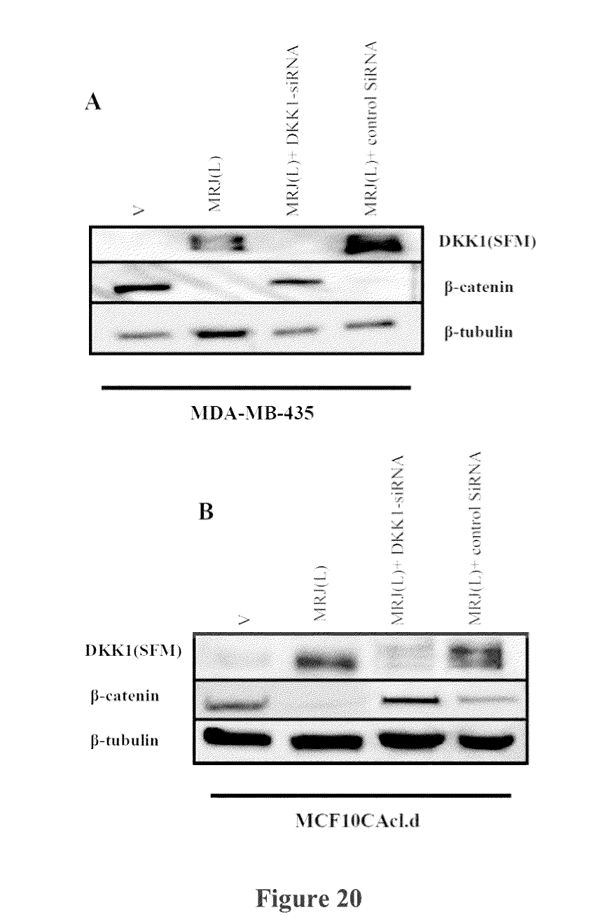

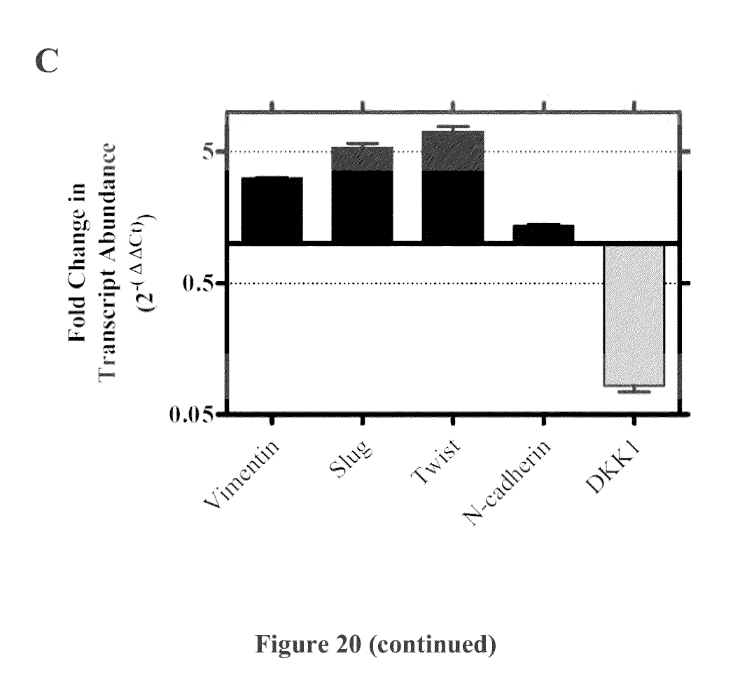

Silencing DKK1 from MRJ(L) expressors was found to restore 3-catenin levels (FIG. 20, panel (A)). MCF10CA1d.c1.1 MRJ(L) expressors also showed comparable restoration of β-catenin levels upon DKK1 silencing (FIG. 20, panel (B)). A slight elevation of β-catenin in the control siRNA transfectants of MCF10CA1d.c1.1MRJ(L) was also observed. Since most of the elevated mesenchymal markers observed are downstream targets of β-catenin transcription (FIG. 16), DKK1 silencing may increase the β-catenin protein levels and result in changes in the transcript levels of the mesenchymal markers. The effect of DKK1 silencing, in MDA-MB-435-MRJ(L), on the EMT markers was examined (FIG. 20, panel (C)). A noticeable upregulation of mesenchymal markers, Vimentin (3.1 fold), Slug (5.2 fold), Twist (7.5 fold) and N-cadherin (1.4 fold) was observed. There was no noticeable change in the β-catenin transcript. However, no significant upregulation was observed of epithelial marker, Keratin 18.

### Example 20

### Silencing of DKK1 from MRJ(L) Expressors Increases their Malignant Activity

MRJ(L) expression reduced malignant activity of cancer cell lines as measured by in vitro assays. Cells ectopically expressing MRJ(L) showed reduced invasion through Matrigel™ and reduced migration using modified Boyden chamber assay, decreased scratch motility (wound healing) assay and less anchorage independence as measured by soft agar colony formation. If the reduced malignant behavior of MRJ(L) expressor cells was due to the inhibition of the Wnt/β-catenin signaling by the elevated DKK1 levels, silencing DKK1 expression will revert their phenotype and make them more malignant.

Colony formation assay: MDA-MB-435 cells stably expressing MRJ(L) were plated in 6 well plates and transfected with DKK1 siRNA and control siRNA using Lipofectamine 2000 (Invitrogen) as per manufacturer's instructions. After 24 h of transfection, the cells were plated into three 10 cm plates in selection medium and were allowed to grow for 12 days. Crystal violet staining was used for visualization of foci. Six different fields were counted for the number of foci and data is represented as average+/−SEM. Soft agar colonization assay: MDA-MB-435 cells (2×10³) treated with DKK1siRNA or control siRNA suspended in 0.35% agar were plated onto a layer of 0.75% bactoagar in DMEM/F12 (10% fetal bovine serum) in six well tissue culture dishes. Visible colonies (>50 cells) were counted after 15 days with the aid of a dissecting microscope. Wound Healing Assay: Cells were cultured to confluence on six-well plates, which were previously externally marked with parallel lines to use as guides for the subsequent photography. The cells were transfected with DKK1 siRNA and control siRNA using Lipofectamine 2000 (Invitrogen) as per manufacturer's instructions. After 24 h of transfection, a central linear wound (perpendicular to the guide lines) was made with a 200 μl sterile pipette tip. Media were changed gently to remove any floating cells. Phase micrographs of the wound cultures were taken at 0 and 16 h (average doubling time is about 20 h). The photographs were analyzed by measuring the distance from the wound edge of the cell sheet at the end or 16 h to the original wound site. Wound healing activity was calculated as the mean distance between edges in 12 independent fields per well. Each test group was assayed in triplicate, and the results are expressed relative to vector control cell migration. Migration assay: Migration assays were conducted using 8 μm polyethylene terpthalate filters (BD Pharmingen, San Diego, Calif., USA), as described previously (Shevde, L. A., et al. (2006) Clin Exp Metastasis 23, 123-133). Cells (treated with DKK1siRNA or control siRNA) which migrated to the lower sides of the trans-well were stained using 0.05% crystal violet and the cell number was counted. Each test group was assayed in triplicate. Four different fields of each insert were photographed at 10× magnification using a Zeiss Axiocam 200 M microscope (Zeiss Axiocam, Carl Zeiss Microimaging Inc. Thornwood, N.Y., USA). Each field was divided into quadrants, and cells in diagonally opposite quadrants were counted.

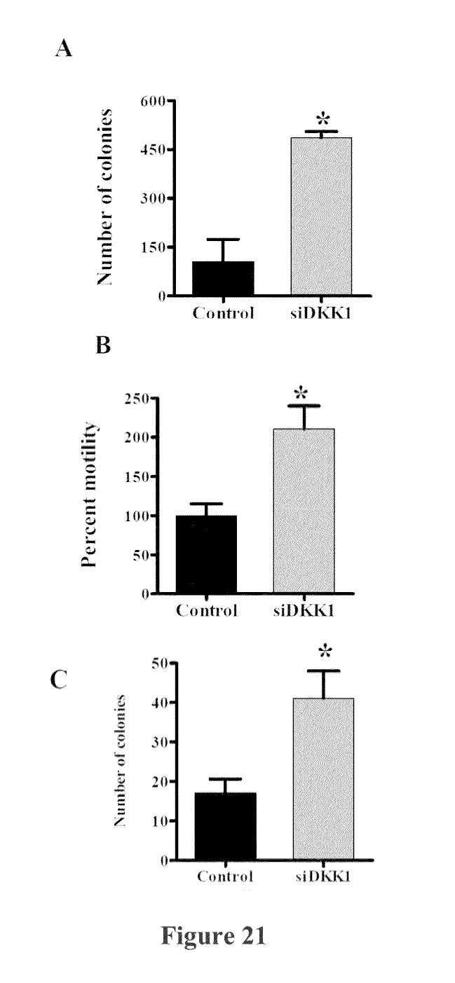

As observed from the data in (FIG. 21, panel (A). 21, panel (B), AND 21, panel (C)), silencing of DKK1 from 435-MRJ(L) increased its motility by about 2.5 fold (FIG. 21, panel (A)) as well as foci formation ability by about 3 fold (FIG. 21, panel (B)). The anchorage independent growth as measured by soft agar colony formation was increased by about 2 fold (FIG. 21, panel (C)).

### Example 21

### Abrogating of MRJ(L) from MCF10A Leads to Activation of Wnt/rβ-Catenin Signaling

MCF10A (immortalized breast epithelial line), expresses noticeably high levels of MRJ(L) as compared to breast cancer cell lines (Mitra, A., et al. (2008) Breast Cancer Res 10, R22). Knockdown of MRJ(L) from MCF10A may activate Wnt/β-catenin signaling. As seen in FIG. 22, panel (A), knockdown of MRJ(L) using shRNA NM_{—}808 increases the β-catenin levels. M50-TOPFlash reporter measures transcription activation by TCF/LEF transcription factors, which is indicative of active Wnt/β-catenin signaling. The activity of Wnt/β-catenin signaling in the MCF10A-MRJ(L) knockdown cells was tested using M50-TOPFlash reporter assays. A modest but significant and reproducible activation of the reporter in the MRJ(L) knockdown cells was observed, indicative of active Wnt/β-catenin signaling (FIG. 22, panel (B)).

To test if the MCF10A cells, which show a mostly epithelial phenotype, show a shift towards mesenchymal phenotype, the changes in transcript levels of some key epithelial markers upon MRJ(L) knockdown from MCF10A cells was evaluated. As seen in FIG. 22, panel (C), the shRNA knockdown of MRJ(L) was observed to accompany a concomitant reduction in DKK1 transcript and downregulation of CDH1 (E-cadherin) transcript indicating loss of epithelial phenotype.

c-Myc is a key oncogenic target of the Wnt/β-catenin signaling.

MRJ(L) knockdown was observed to increase c-Myc transcript in MCF10A by 2.5 fold, suggesting activation of Wnt/β-catenin signaling. However, no change in the levels of KRT18 (Keratin 18), an epithelial marker that gets upregulated at protein levels upon MRJ(L) overexpression was observed.

EMT is one of the series of molecular changes in cancer progression, which may affect prognosis of cancers such as breast cancer (Jo, M., et al. (2009) J Biol Chem 284, 22825-22833; Lester, R. D., et al. (2007) J Cell Biol 178, 425-436; and Lame, L., and Bellacosa, A. (2005) Oncogene 24, 7443-7454). Several signaling pathways have been implicated to play a role in EMT of cancer cells, including Wnt/β-catenin (Kalluri, R., and Weinberg, R. A. (2009) J Clin Invest 119, 1420-1428; and Kalluri, R., and Neilson, E. G. (2003) J Clin Invest 112, 1776-1784). Of all the EMT related signaling changes that accompanied MRJ(L) overexpression, reduced levels of β-catenin protein was most consistent across the different cell types examined. This reduction led to significant inhibition of the Wnt/β-catenin signaling in the MRJ(L) expressors (FIG. 18, panels (A)-(C)). β-catenin is a major player in the Wnt signaling pathway, as well as, a regulator of EMT in cancer cells and possibly in maintaining a metastable phenotype (Huber, M. A., et al. (2005) Curr Opin Cell Biol 17, 548-558; and Jiang, Y. G., et al. (2007) Int J Urol 14, 1034-1039). MRJ(L) expression may promote the degradation of β-catenin in a proteasome dependent manner (FIG. 17). The known pathways that drive the degradation of β-catenin are initiated by inhibition of Wnt ligands or their receptors by their inhibitors like sFRPs, DKKs etc (Kawano, Y., and Kypta, R. (2003) J Cell Sci 116, 2627-2634; and Moon, R. T., et al. (1997) Cell 88, 725-728). Breast cancer cells show relatively lower mRNA levels of DKK1 as compared to non-tumorigenic MCF10A cells (FIG. 19, panel (A)); moreover the expression profiles of DKK1 paralleled that of MRJ(L). Analysis of MRJ(L) overexpressors showed increased levels of secreted DKK1, due to the transcriptional upregulation of DKK1 (FIG. 19, panel (C)), which possibly caused reduced β-catenin protein levels (FIG. 19, panel (B)). Knockdown of upregulated endogenous DKK1 by treatment of 435-MRJ(L) with DKK1-siRNA restored β-catenin levels in MRJ(L). This confirmed the role of MRJ(L) upregulated DKK1 in degrading β-catenin (FIG. 20, panels (A)-(C)). Enhancement in motility, increased foci formation as well as increased soft agar colonization was seen in DKK1 silenced MDA-MB-435 cells indicating that the MRJ(L) upregulated DKK1 contributed to the reduction of malignant activity.

DKK1 loss has been correlated with aggressive breast cancer and is reported to be frequently inactivated by epigenetic inactivation (Suzuki, H., et al. (2008) Br J Cancer 98, 1147-1156). DKK1 is implicated as a player in inhibiting malignant behavior of breast cancer (Fillmore, R. A., et al. (2009) Int J Cancer 125, 556-564; Cowling, V. H., and Cole, M. D. (2007) Cell Cycle 6; Cowling, V. H., et al. (2007) Mol Cell Biol 27, 5135-5146; DiMeo, T. A., et al. (2009) Cancer Res 69, 5364-5373; and Mikheev, A. M., et al. (2007) Breast Cancer Res Treat). Stable expression of DKK1 in breast cancer cells has been reported to increase expression of Keratin18 levels concomitantly with a decrease in β-catenin levels (Bafico, A., et al. (2004) Cancer Cell 6, 497-506). Recently interesting studies by DiMeo et al. showed that silencing LRP6, a Wnt co-receptor that is blocked by DKK1, reduced the capacity of cancer cells to self-renew and seed tumors in vivo and also resulted in the re-expression of breast epithelial differentiation markers and repression of EMT transcription factors SLUG and TWIST (DiMeo, T. A., et al. (2009) Cancer Res 69, 5364-5373).

The Wnt signaling pathway has been reported to be associated to the invasive ductal carcinoma of the breast (Prasad, C. P., et al. (2007) Oncology 73, 112-117). Stabilized β-catenin induces hyperplasia and mammary tumors in mice (Hatsell, S., et al. (2003) J Mammary Gland Biol Neoplasia 8, 145-158). Activation of Wnt/β-catenin signaling components in mammary epithelium induces trans-differentiation (Miyoshi, K., et al. (2002) Oncogene 21, 5548-5556). In melanoma as well, Wnt signaling has been shown to be involved in inhibition of metastasis suppressors and activation of EMT (Dissanayake, S. K., et al (2007) J Biol Chem 282, 17259-17271). MRJ(L) has a role in up-regulating DKK1, leading to inhibition of the Wnt/β-catenin signaling pathway. This inhibition could be one of the ways by which MRJ(L) reduces tumorigenicity and metastasis of aggressive cancer cells.

### Example 22

### Identifying Sequences with Inducible Nuclear Localization Activity

A nucleic acid sequence encoding a portion of the MRJ(S) polypeptide or MRJ(L) polypeptide is selected. The nucleic acid is operably linked to a E-GFP reporter gene in an expression vector. The expression vector is transfected into COS7 cells. The location of the expressed fusion gene is evaluated. The transfected cells are stimulated by heat shock. The location of the expressed fusion gene is evaluated. Translocation of the expressed fusion gene from a cytoplasmic location to a nuclear location on stimulation can be indicative that the selected nucleic acid sequence includes elements with inducible nuclear localization activity.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

1.-60. (canceled)

61. A method for treating a cancer in a subject comprising:
(a) measuring the expression level of a nucleic acid encoding MRJ(L) or the expression level of MRJ(L) protein in a sample obtained from a cancer in the subject; and
(b) administering a treatment regimen known to be appropriate for an aggressive cancer to the subject based on the expression level of the nucleic acid encoding MRJ(L) or the expression level of MRJ(L) protein in the sample.

62. The method of claim 61, further comprising comparing said expression level of said nucleic acid encoding MRJ(L) or the expression level of said MRJ(L) protein in said sample to the expression level of said nucleic acid encoding MRJ(L) or the expression level of said MRJ(L) protein in normal tissue, tissue from a known stage of cancer, or cancerous tissue with a known metastatic potential.

63. The method of claim 62, wherein said treatment regimen known to be appropriate for an aggressive cancer is administered to said subject if said sample has a decreased level of expression of said MRJ(L) protein or said nucleic acid encoding said MRJ(L) protein.

64. The method of claim 62, further comprising measuring the level of expression of at least one marker in addition to MRJ(L) in said sample.

65. The method of claim 64, wherein said at least one marker in addition to MRJ(L) comprises DKK-1.

66. The method of claim 65, wherein said treatment regimen known to be appropriate for an aggressive cancer is administered to said subject if said sample has a decrease in the level of expression of said at least one marker in addition to MRJ(L).

67. The method of claim 64, wherein said at least one marker in addition to MRJ(L) comprises osteopontin, osteonectin, zinc binding α-2-glycoprotein, VGF nerve growth factor, nucleophosmin, N-cadherin, vimentin, twist and β-catenin.

68. The method of claim 67, wherein said treatment regimen known to be appropriate for an aggressive cancer is administered to said subject if said sample has an increase in the level of expression of said at least one marker in addition to MRJ(L).

69. The method of claim 61, wherein the cancer comprises a cancer selected from breast cancer, skin cancer, or ovarian cancer.

70. The method of claim 69, wherein the breast cancer comprises an infiltrating ductal carcinoma.

71. The method of claim 61, wherein the treatment regimen known to be appropriate for an aggressive cancer is selected from the group consisting of surgery, radiotherapy, chemotherapy, and hormone therapy.

72. A method of selectively treating a cancer, comprising:
(a) selecting a subject for a treatment regimen known to be appropriate for an aggressive cancer on the basis of the subject having a decreased expression level of a nucleic acid encoding MRJ(L) or a decreased expression level of MRJ(L) protein in a sample obtained from a cancer in the subject compared to the expression level of said nucleic acid encoding MRJ(L) or the expression level of said MRJ(L) protein in normal tissue, tissue from a known stage of cancer, or cancerous tissue with a known metastatic potential; and
(b) selectively administering the treatment regimen known to be appropriate for an aggressive cancer to the subject.

73. The method of claim 72, wherein the subject is selected on the basis of having an increased level of expression of at least one marker in addition to MRJ(L), wherein said marker in addition to MRJ(L) is selected from the group consisting of osteopontin, osteonectin, zinc binding α-2-glycoprotein, VGF nerve growth factor, nucleophosmin, N-cadherin, vimentin, twist and β-catenin.

74. The method of claim 72, wherein the subject is selected on the basis of having a decreased level of expression of DKK1 in addition to the decreased level of expression of MRJ(L).

75. The method of claim 72, wherein the treatment regimen known to be appropriate for an aggressive cancer is selected from the group consisting of surgery, radiotherapy, chemotherapy, and hormone therapy.

76. The method of claim 72, wherein the cancer comprises breast cancer, skin cancer, or ovarian cancer.

77. The method of claim 76, wherein the breast cancer comprises an infiltrating ductal carcinoma.

78. A method of treating cancer in a subject comprising:
(a) measuring the expression level of a nucleic acid encoding MRJ(L) or the expression level of MRJ(L) protein in a sample obtained from a cancer in the subject;
(b) comparing the expression level of the nucleic acid encoding MRJ(L) or the expression level of the MRJ(L) protein in the sample to the expression level of the nucleic acid encoding MRJ(L) or the expression level of the MRJ(L) protein in normal tissue, tissue from a known stage of cancer, or cancerous tissue with a known metastatic potential; and
(c) administering a treatment regimen known to be appropriate for an aggressive cancer to the subject based on a decreased expression level of the nucleic acid encoding MRJ(L) or a decreased expression level of MRJ(L) protein between the sample and normal tissue, tissue from a known stage of cancer, or cancerous tissue with a known metastatic potential.

79. The method of claim 78, comprising:
measuring the expression level of at least one marker in addition to MRJ(L) in the sample, the additional marker selected from the group consisting of osteopontin, osteonectin, zinc binding α-2-glycoprotein, VGF nerve growth factor, nucleophosmin, N-cadherin, vimentin, twist and β-catenin; and
comparing the expression level of the additional marker with the expression level of the additional marker in normal tissue, tissue from a known stage of cancer, or cancerous tissue with a known metastatic potential;
wherein administration of the treatment regimen known to be appropriate for an aggressive cancer is further based on an increased expression level of the additional marker between the sample and normal tissue, tissue from a known stage of cancer, or cancerous tissue with a known metastatic potential.

80. The method of claim 78, comprising
measuring the expression level of DKK-1 in the sample; and
comparing the expression level of DKK-1 with the expression level of DKK-1 in normal tissue, tissue from a known stage of cancer, or cancerous tissue with a known metastatic potential;
wherein administration of the treatment regimen known to be appropriate for an aggressive cancer is further based on a decreased expression level of DKK-1 between the sample and normal tissue, tissue from a known stage of cancer, or cancerous tissue with a known metastatic potential.

81. The method of claim 78, wherein the treatment regimen known to be appropriate for an aggressive cancer is selected from the group consisting of surgery, radiotherapy, chemotherapy, and hormone therapy.

82. The method of claim 78, wherein the cancer is breast cancer, skin cancer, or ovarian cancer.

83. The method of claim 82, wherein the breast cancer comprises an infiltrating ductal carcinoma.
